(19) United States
(12) Patent Application Publication
(10) Pub. No.: US 2015/0024966 A1
(43) Pub. Date: Jan. 22, 2015

(54) MESENCHYMAL STEM CELLS FOR IN VITRO MODELING AND CELL-BASED THERAPY OF HUMAN DISEASES AND BANKS THEREOF

(71) Applicants: BrainStem Biotec Ltd., Tel-Aviv (IL); Henry Ford Health System, Detroit, MI (US)

(72) Inventors: Chaya Brodie, Southfield, MI (US); Shimon Slavin, Tel-Aviv (IL)

(21) Appl. No.: 14/380,174

(22) PCT Filed: Feb. 21, 2013
(86) PCT No.: PCT/IB2013/051428

§ 371 Date: Aug. 21, 2014

Related U.S. Application Data
(60) Provisional application No. 61/601,619, filed on Feb. 22, 2012.
(60) Provisional application No. 61/601,596, filed on Feb. 22, 2012.
(60) Provisional application No. 61/601,624, filed on Feb. 22, 2012.

(51) Int. Cl. G01N 33/50

(52) U.S. Cl. CPC G01N 33/5008; G01N 2500/04; G01N 2500/10; G01N 2333/435
USPC 506/10

(57) ABSTRACT

A method of qualifying a mesenchymal stem cell (MSC) population is disclosed. The method comprises:
(a) ex vivo differentiating a population of mesenchymal stem cells originating from the subject towards a first lineage-specific cell, the first lineage-specific cell being associated with a brain disease;
(b) ex vivo differentiating a population of mesenchymal stem cells originating from a healthy subject towards the first lineage-specific cell;
(c) comparing an effect of the first lineage specific cell derived from the subject with an effect of the first lineage specific cell derived from the healthy subject on a second lineage specific cell associated with the brain disease, wherein a difference in the effect above or below a predetermined level is indicative of a qualification of a mesenchymal stem cell population for cell therapy of said brain disease.

### RELATED APPLICATION

This application claims the benefit of priority of U.S. Provisional Patent Application Nos. 61/601,619 filed Feb. 22, 2012, 61/601,596 filed Feb. 22, 2012 and 61/601,624 filed Feb. 22, 2012, the contents of which are incorporated herein by reference in their entirety.

### FIELD AND BACKGROUND OF THE INVENTION

The present invention, in some embodiments thereof, relates to mesenchymal stem cell populations for in vitro modeling of human diseases and methods of selecting suitable mesenchymal stem cell populations for the treatment of such diseases.

Current approaches to target-driven drug discovery typically involve screening a large compound library against a single enzyme or receptor, followed by prioritization of hits based on chemical tractability and optimization through medicinal chemistry to achieve potency and selectivity. Typically, at a later stage in preclinical development, candidate compounds are tested in a relevant primary cell system or animal model, often with disappointing results. In contrast to target-directed biochemical screens, the use of cell-based phenotypic assays has increased, driven by advances in high-content image processing and the appreciation that cell-based screens represent a more physiological system for high-throughput screening and lead optimization.

Cell-based assays have advantages over single-target biochemical assays as they simultaneously confirm cell permeability and tolerable toxicity, but they have many associated limitations. The lack of a known target in cell-based screens hinders the ability to determine the chemical groups responsible for biological activity through structure-activity relationship studies, and requires purely empirical attempts to optimize drug-like properties through trial and error. Although there is a compelling rationale for the use of human primary (preferably disease-bearing cells) for drug screening, lead discovery and optimization, most primary cells are difficult to access and have a finite lifespan in culture. Adaptation of human primary cells to immortal growth in culture—a requisite for their use in cell-based screens typically entails selection for genetic alterations that may influence the cell's response to drugs, thus compromising the fidelity of drug screens and counterscreens.

Although it has been possible to successfully generate and differentiate disease patient-derived iPS to a variety of lineage specific cells (e.g. iPS cells from ALS patients have been differentiated into motor neurons), limitations for the use of iPS cells include the expression of embryonic genes that may introduce erroneous genotypic and phenotypic characteristics, and the inability to directly apply the results of these studies to the clinic since differentiated iPS cannot yet be used clinically.

Mesenchymal stem cells (MSCs) are a heterogeneous population of stromal cells isolated from multiple species, residing in most connective tissues including bone marrow, adipose, umbilical cord, placenta, amniotic fluid and perivascular tissues. MSCs can differentiate into cells of the mesenchymal lineage, such as bone, cartilage and fat but, under certain circumstances, have been reported to acquire the phenotype of cells of the endodermal and neuroectodermal lineage, suggesting some potential for “transdifferentiation”. Within the bone marrow these cells are tightly intermingled with and support hematopoiesis and the survival of hematopoietic stem cells in acquiescent state (Meuleman N et al., Stem Cells Dev. 18(9):1247-52, 2009).

Recent reports have demonstrated that MSCs also have the potential to differentiate into functional cells of the central nervous system including neurons, astrocytes and oligodendrocytes. MSCs have been shown to exert therapeutic effects in a variety of neurological diseases and dysfunctions in experimental animal models and more recently in pilot clinical trials. Their effects have been mainly attributed to immunosuppressive and neuroprotective functions. However, some studies demonstrated that differentiation of these cells increased their therapeutic effect in various instances. Therefore, MSC-derived cells have a great potential as an easily accessible source of cells for treatment of inflammatory and neurodegenerative disorders including Amyotrophic lateral sclerosis (ALS), Alzheimer's disease, Rett syndrome, Multiple Sclerosis and Parkinson's disease aiming for both cell mediated control of disease activity as well as regeneration of damaged or lost functions.

### SUMMARY OF THE INVENTION

According to an aspect of some embodiments of the present invention there is provided a method of qualifying a mesenchymal stem cell (MSC) population, the method comprising:

(a) ex vivo differentiating a population of mesenchymal stem cells originating from the subject towards a first lineage-specific cell, the first lineage-specific cell being associated with a brain disease;

(b) ex vivo differentiating a population of mesenchymal stem cells originating from a healthy subject towards the first lineage-specific cell;

(c) comparing an effect of the first lineage specific cell derived from the subject with an effect of the first lineage specific cell derived from the healthy subject on a second lineage specific cell associated with the brain disease, wherein a difference in the effect above or below a predetermined level is indicative of a qualification of a mesenchymal stem cell population.

According to an aspect of some embodiments of the present invention there is provided a method of qualifying a population of mesenchymal stem cells (MSCs), the method comprising analyzing a function and/or morphology of a lineage-specific cell differentiated from the population of the MSCs, wherein an alteration in the function or morphology of the lineage-specific cell compared to a control lineage specific cell is indicative of a qualification of the MSCs.

According to an aspect of some embodiments of the present invention there is provided a method of selecting an agent for the treatment of a brain disease, the method comprising analyzing an ability of a population of MSCs isolated from a patient with a brain disorder to differentiate towards a lineage-specific cell associated with the brain disorder in a presence and absence of the agent, wherein an improvement of a function or morphology of the lineage specific cell as compared to an identical lineage specific cell derived from a control population of MSCs in the presence of the agent is indicative that the agent may be used for the treatment of the brain disease.

According to an aspect of some embodiments of the present invention there is provided a method of producing a mesenchymal stem cell (MSC) bank comprising: harvesting undifferentiated mesenchymal stem cells from a plurality of subjects which have a brain disorder to obtain a plurality of separate cell populations; differentiating the mesenchymal stem cells towards a lineage specific cell; storing the undifferentiated mesenchymal stem cells and the lineage specific cells thereby producing a mesenchymal stem cell bank.

According to an aspect of some embodiments of the present invention there is provided a stem cell bank comprising multiple MSC populations isolated from patients with a brain disorder, each individually disposed within separate containers.

According to some embodiments of the invention, the qualifying is to ascertain whether the MSC population is suitable for autologous cell replacement therapy for the treatment of the brain disease of a subject.

According to some embodiments of the invention, the method further comprises comparing an effect of a third lineage specific cell derived from the subject with an effect of the third lineage specific cell derived from the healthy subject on the first and/or the second lineage specific cell.

According to some embodiments of the invention, the second lineage specific cell has been ex vivo differentiated from a population of mesenchymal stem cells.

According to some embodiments of the invention, the brain disease is selected from the group consisting of a neurodegenerative disorder, a neuroinflammatory disorder and a prion-mediated disorder.

According to some embodiments of the invention, the neurodegenerative disease is selected from the group consisting of Amyotrophic Lateral Sclerosis (ALS), Parkinson's disease, Alzheimer's disease, Huntington's disease and Rett syndrome.

According to some embodiments of the invention, the brain disease is multiple sclerosis, ALS, Parkinson's disease, Alzheimer's disease, Huntington's disease, Rett syndrome, autism spectrum disease, aspergers syndrome, dementia and cerebral atrophy, multiple system atrophy, Creutzfeldt-Jakob disease, pontocerebellar hypoplasia, corticobasal degeneration, progressive supranuclear palsy, spinocerebellar atrophy, spinal & bulbar muscular atrophy, Charcot Marie Tooth disease, giant axonal neuropathy, Canavan disease, Friedreich's and other ataxias, epilepsy, early infantile encephalopathy, hereditary spastic paraplegia, amyloidosis of the central nervous system, tourette syndrome, Shy-Drager syndrome, Meniere's syndrome, Alpers disease, familial dysautonomia, dyslexia, Wernig-Hoffman disease, tuberous sclerosis, and neurofibromatosis.

According to some embodiments of the invention, the brain disease is a psychiatric disease.

According to some embodiments of the invention, the method further comprises analyzing for a genetic aberration of the MSCs originating from the subject, the genetic aberrations being associated with a brain disease.

According to some embodiments of the invention, the population of MSCs is derived from a subject having a brain disease.

According to some embodiments of the invention, the qualifying is to ascertain whether the MSCs are suitable for autologous treatment of a brain disease of a subject.

According to some embodiments of the invention, the method further comprises analyzing for a genetic aberration of the MSCs, the genetic aberrations being associated with a brain disease.

According to some embodiments of the invention, the function of the lineage-specific cell comprises a response to a microglia cell.

According to some embodiments of the invention, the brain disease is a neurodegenerative disorder.

According to some embodiments of the invention, the neurodegenerative disease is selected from the group consisting of Amyotrophic Lateral Sclerosis (ALS), Parkinson's disease, Alzheimer's disease, Huntington's disease and Rett syndrome.

According to some embodiments of the invention, the brain disease is a psychiatric disease.

According to some embodiments of the invention, the brain disease is Amyotrophic Lateral Sclerosis (ALS), the lineage specific cells are selected from the group consisting of motor neurons, skeletal muscle and astrocytes.

According to some embodiments of the invention, when the brain disease is Parkinson's disease, the lineage specific cells are selected from the group consisting of astrocytes and dopaminergic neurons.

According to some embodiments of the invention, when the brain disease is Rett syndrome, the lineage specific cells are selected from the group consisting of astrocytes and neurons.

According to some embodiments of the invention, the method further comprises characterizing the undifferentiated cell populations to obtain at least one predetermined characteristic for each of the undifferentiated cell population and cataloguing the undifferentiated cell populations according to the at least one predetermined characteristic.

According to some embodiments of the invention, the method further comprises characterizing the differentiated cell populations to obtain at least one predetermined characteristic for each differentiated cell population and cataloguing the differentiated cell populations according to the at least one predetermined characteristic.

According to some embodiments of the invention, the characterizing comprises analyzing an ability of the MSCs to differentiate towards a lineage-specific cell associated with the disease.

According to some embodiments of the invention, the characterizing comprises analyzing for a genetic aberration of the MSCs, the genetic aberrations being associated with the disease.

According to some embodiments of the invention, the storing comprises disposing the cell populations in separate containers under freezing conditions.

According to some embodiments of the invention, the cataloguing comprises inputting information about the predetermined characteristic of a given cell population into a database computer.

According to some embodiments of the invention, the predetermined characteristic comprises disease state information of the subject.

According to some embodiments of the invention, the bank comprises lineage specific cells differentiated from the MSC populations, wherein the lineage specific cells are associated with the brain disorder.

According to some embodiments of the invention, the bank further comprises a catalogue which comprises information about a predetermined characteristic of the MSC populations.

According to some embodiments of the invention, the predetermined characteristic comprises a differentiation potential of the MSC populations.

According to some embodiments of the invention, the predetermined characteristic comprises a genetic characteristic.

According to some embodiments of the invention, the catalogue is at least one database computer unit comprising at least one processing module and at least one memory device into which predetermined characteristic information of the multiple cell populations is inputted, and at least program code module for causing predetermined characteristic information to be displayed onto a display communicatingly connected to the database computer upon request.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a pictorial representation of the cells used in exemplary assays of the present invention. This figure serves as a key for FIGS. 2-4.

FIG. 2 is a pictorial representation of an exemplary assay in order to determine if a mesenchymal stem cell population is suitable for treating ALS.

FIG. 3 is a pictorial representation of an exemplary assay in order to determine if a mesenchymal stem cell population is suitable for treating Parkinson's disease.

FIG. 4 is a pictorial representation of an exemplary assay in order to determine if a mesenchymal stem cell population is suitable for treating Rett syndrome.

FIG. 5 is a pictorial representation is a pictorial representation of the ALS model of a particular embodiment of the present invention. The ability to differentiate MSCs to neural stem cell like, glial progenitors and astrocytes as well as to motor neuron progenitors and motor neuron-like cells allows the development of a novel ALS in vitro model, which can be used to analyze the functions of astrocytes derived from healthy and ALS donors and their interactions with motor neurons derived from the same donors. These cells can be further employed to identify novel mechanisms of neuronal cell death, neurotoxicity and neuroprotection, to identify novel therapeutic targets and to serve as a unique system for drug screening. Moreover, since these cells can be employed in cell therapy of ALS, the results of these studies have direct translational and clinical implications.

FIG. 6 is a pictorial representation of the ALS model of a particular embodiment of the present invention. The results of the in vivo studies will supplement the in vitro studies and will demonstrate whether the different types of the differentiated MSCs have therapeutic beneficial over the unmodified cells.

FIG. 7 is a pictorial representation of the ALS model of a particular embodiment of the present invention. Based on the in vitro and pre-clinical studies the therapeutic potential of the different types and sources of MSCs can be evaluated. Autologous, haploidentical, or “off-the-shelf” cells (placenta or cord-derived MSCs) can be used as either unmodified or differentiated cells.

FIG. 8A is a bar graph illustrating that the conditioned media of MSCs derived from healthy control did not exert cell death of motor neuron cultures.

FIG. 8B is a bar graph illustrating that the conditioned media of astrocytes differentiated from MSCs derived from ALS patients did exert cell death of motor neuron cultures.

FIG. 9 is a bar graph illustrating that MSC-derived astrocyte like cells expressing a SOD1 G93A mutant induced cell death of motor neurons, whereas unmodified MSCs did not have a significant effect on motor neurons. MSC-derived astrocyte like cells having a wild-type SOD1 did not induce cell death of motor neurons.

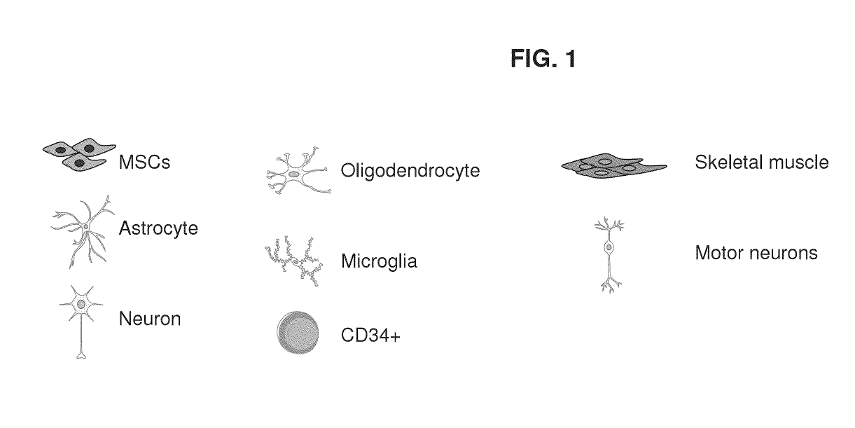

### DESCRIPTION OF SPECIFIC EMBODIMENTS OF THE INVENTION

The present invention, in some embodiments thereof, relates to mesenchymal stem cell populations for in vitro modeling of human diseases and methods of selecting suitable mesenchymal stem cell populations for the treatment of such diseases.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Most of our current knowledge about cellular phenotypes in neurodevelopmental and neurodegenerative diseases in humans has been gathered from studies in postmortem brain tissues. These samples often represent the end-stage of the disease and therefore are not always a fair representation of how the disease developed. Moreover, under these circumstances, the pathology observed could be a secondary effect rather than the authentic disease cellular phenotype. Likewise, rodent models available do not always recapitulate the pathology from human diseases.

Accordingly, both research into the pathophysiological mechanisms of human disease, and the development of targeted therapies have been hindered by a lack of predictive disease models that can be experimentally manipulated in vitro.

For example, the SOD mouse which is used as an experimental model for studying Amyotrophic lateral sclerosis (ALS) represents an acceptable experimental model for studying novel approaches for the treatment of ALS, even though the relevance of SOD-mice to the familial ALS (fALS) remains questionable. Moreover, no experimental animal models exist for studying sporadic ALS (sALS) which represent the large majority of the cases of ALS.

Similarly, animal models that harbor genetic lesions that mimic inherited forms of human PD, such as homozygous deletions in parkin or over-expression of α-synuclein have failed to recapitulate the loss of dopaminergic neurons.

The optimal approach would be to study the mechanisms of cell degeneration using cells derived from patients of neurodegenerative diseases; however it is practically impossible to isolate all the relevant cell types from these patients.

Although it has been possible to successfully generate and differentiate disease patient-derived iPS to a variety of lineage specific cells (e.g. iPS cells from ALS patients have been differentiated into motor neurons), limitations for the use of iPS cells include the expression of embryonic genes that may introduce erroneous genotypic and phenotypic characteristics, and the inability to directly apply the results of these studies to practice since differentiated iPS cannot yet be used clinically.

With the ability to differentiate mesenchymal stem cells (MSCs) into various cells associated with neurodegenerative diseases, including for example, motor neurons, oligodendrocytes and astrocytes, the present inventors propose the use of mesenchymal stem cells derived from diseased subjects to model neurodegenerative diseases. Such models would enhance the basic understanding of the disease, allow for selection of mesenchymal stem cell populations for the treatment of particular diseases and would pave the way for their application in drug screening and optimization. Knowledge gleaned from the models derived from diseased MSCs would allow for the generation of models derived from healthy MSCs. For example, if it was found that a particular gene was down-regulated in a neuronal type cell generated from the diseased MSCs, that same gene could be down-regulated (by gene knock-out, siRNA, miRNA etc) in healthy MSCs and following differentiation into the neuronal cell type would serve as a model for the disease.

In addition, MSCs derived from known genotypes can be used to assess the variability in response to drugs or examine the molecular mechanisms underlying genetic diseases.

Thus, according to a first aspect of the present invention there is provided a method of qualifying a mesenchymal stem cell population, the method comprising:

(a) ex vivo differentiating a population of mesenchymal stem cells originating from the subject towards a first lineage-specific cell, the first lineage-specific cell being associated with a brain disease;

(b) ex vivo differentiating a population of mesenchymal stem cells originating from a healthy subject towards the first lineage-specific cell;

(c) comparing an effect of the first lineage specific cell derived from the subject with an effect of the first lineage specific cell derived from the healthy subject on a second lineage specific cell associated with the brain disease, wherein a difference in the effect above or below a predetermined level is indicative of a qualification of the mesenchymal stem cell population.

The term “mesenchymal stem cell” or “MSC” is used interchangeably for adult cells which are not terminally differentiated, which can divide to yield cells that are either stem cells, or which, irreversibly differentiate to give rise to cells of a mesenchymal cell lineage, e.g., adipose, osseous, cartilaginous, elastic and fibrous connective tissues, myoblasts) as well as to tissues other than those originating in the embryonic mesoderm (e.g., neural cells) depending upon various influences from bioactive factors such as cytokines.

Methods of isolating, purifying and expanding mesenchymal stem cells (MSCs) are known in the arts and include, for example, those disclosed by Caplan and Haynesworth in U.S. Pat. No. 5,486,359 and Jones E. A. et al., 2002, Isolation and characterization of bone marrow multipotential mesenchymal progenitor cells, Arthritis Rheum. 46(12): 3349-60.

Mesenchymal stem cells may be isolated from various tissues including but not limited to bone marrow, peripheral blood, blood, placenta (both chorionic and/or amniotic), cord blood, umbilical cord, amniotic fluid and from adipose tissue.

A method of isolating mesenchymal stem cells from peripheral blood is described by Kassis et al [Bone Marrow Transplant. 2006 May; 37(10):967-76]. A method of isolating mesenchymal stem cells from placental tissue is described by Zhang et al [Chinese Medical Journal, 2004, 117 (6):882-887]. Methods of isolating and culturing adipose tissue, placental and cord blood mesenchymal stem cells are described by Kern et al [Stem Cells, 2006; 24:1294-1301].

According to a preferred embodiment of this aspect of the present invention, the mesenchymal stem cells are human.

According to another embodiment of this aspect of the present invention, the mesenchymal stem cells are isolated from newborn humans.

Bone marrow can be isolated from the iliac crest of an individual by aspiration. Low-density BM mononuclear cells (BMMNC) may be separated by a FICOL-PAQUE density gradient or by elimination of red blood cells using Hetastarch (hydroxyethyl starch). Preferably, mesenchymal stem cell cultures are generated by diluting BM aspirates (usually 20 ml) with equal volumes of Hank's balanced salt solution (HBSS; GIBCO Laboratories, Grand Island, N.Y., USA) and layering the diluted cells over about 10 ml of a Ficoll column (Ficoll-Paque; Pharmacia, Piscataway, N.J., USA). Following 30 minutes of centrifugation at 2,500×g, the mononuclear cell layer is removed from the interface and suspended in HBSS. Cells are then centrifuged at 1,500×g for 15 minutes and resuspended in a complete medium (MEM, α medium without deoxyribonucleotides or ribonucleotides; GIBCO); 20% fetal calf serum (FCS) derived from a lot selected for rapid growth of MSCs (Atlanta Biologicals, Norcross, Ga.); 100 units/ml penicillin (GIBCO), 100 μg/ml streptomycin (GIBCO); and 2 mM L-glutamine (GIBCO). Resuspended cells are plated in about 25 ml of medium in a 10 cm culture dish (Corning Glass Works, Corning, N.Y.) and incubated at 37° C. with 5% humidified CO₂. Following 24 hours in culture, nonadherent cells are discarded, and the adherent cells are thoroughly washed twice with phosphate buffered saline (PBS). The medium is replaced with a fresh complete medium every 3 or 4 days for about 14 days. Adherent cells are then harvested with 0.25% trypsin and 1 mM EDTA (Trypsin/EDTA, GIBCO) for 5 min at 37° C., replated in a 6-cm plate and cultured for another 14 days. Cells are then trypsinized and counted using a cell counting device such as for example, a hemocytometer (Hausser Scientific, Horsham, Pa.). Cultured cells are recovered by centrifugation and resuspended with 5% DMSO and 30% FCS at a concentration of 1 to 2×10⁶ cells per ml. Aliquots of about 1 ml each are slowly frozen and stored in liquid nitrogen.

Adipose tissue-derived MSCs can be obtained by liposuction and mononuclear cells can be isolated manually by removal of the fat and fat cells, or using the Celution System (Cytori Therapeutics) following the same procedure as described above for preparation of MSCs.

According to one embodiment the populations are plated on polystyrene plastic surfaces (e.g. in a flask) and mesenchymal stem cells are isolated by removing non-adherent cells. Alternatively mesenchymal stem cell may be isolated by FACS using mesenchymal stem cell markers.

Preferably the MSCs are at least 50% purified, more preferably at least 75% purified and even more preferably at least 90% purified.

To expand the mesenchymal stem cell fraction, frozen cells are thawed at 37° C., diluted with a complete medium and recovered by centrifugation to remove the DMSO. Cells are resuspended in a complete medium and plated at a concentration of about 5,000 cells/cm². Following 24 hours in culture, nonadherent cells are removed and the adherent cells are harvested using Trypsin/EDTA, dissociated by passage through a narrowed Pasteur pipette, and preferably replated at a density of about 1.5 to about 3.0 cells/cm². Under these conditions, MSC cultures can grow for about 50 population doublings and be expanded for about 2000 fold [Colter D C., et al. Rapid expansion of recycling stem cells in cultures of plastic-adherent cells from human bone marrow. Proc Natl Acad Sci USA. 97: 3213-3218, 2000].

MSC cultures utilized by some embodiments of the invention preferably include three groups of cells which are defined by their morphological features: small and agranular cells (referred to as RS-1, herein below), small and granular cells (referred to as RS-2, herein below) and large and moderately granular cells (referred to as mature MSCs, herein below). The presence and concentration of such cells in culture can be assayed by identifying a presence or absence of various cell surface markers, by using, for example, immunofluorescence, in situ hybridization, and activity assays.

When MSCs are cultured under the culturing conditions of some embodiments of the invention they exhibit negative staining for the hematopoietic stem cell markers CD34, CD11B, CD43 and CD45. A small fraction of cells (less than 10%) are dimly positive for CD31 and/or CD38 markers. In addition, mature MSCs are dimly positive for the hematopoietic stem cell marker, CD117 (c-Kit), moderately positive for the osteogenic MSCs marker, Stro-1 [Simmons, P. J. & Torok-Storb, B. (1991). Blood 78, 5562] and positive for the thymocytes and peripheral T lymphocytes marker, CD90 (Thy-1). On the other hand, the RS-1 cells are negative for the CD117 and Strol markers and are dimly positive for the CD90 marker, and the RS-2 cells are negative for all of these markers.

As used herein, the phrase “brain disease” refers to any disorder, disease or condition of the central nervous system including neurodegenerative disorders and psychiatric disorders.

Additional representative examples of brain diseases or disorders include, but are not limited to, a pain disorder, a motion disorder, a dissociative disorder, a mood disorder, an affective disorder, a neurodegenerative disease or disorder and a convulsive disorder.

More specific examples of such conditions include, but are not limited to, Parkinson's, ALS, Multiple Sclerosis, Huntingdon's disease, autoimmune encephalomyelitis, diabetic neuropathy, glaucatomus neuropathy, macular degeneration, action tremors and tardive dyskinesia, panic, anxiety, depression, alcoholism, insomnia, manic behavior, Alzheimer's, epilepsy, Rett Syndrome and autism.

According to a particular embodiment, the disease is Amyotrophic Lateral Sclerosis (ALS), Parkinson's disease, Alzheimer's disease, Huntington's disease or Rett syndrome.

According to one embodiment, the qualifying is in order to ascertain whether the MSC population is suitable for autologous cell replacement therapy for the treatment of a brain disease of a subject.

According to another embodiment, the qualifying is in order to ascertain whether it is beneficial to genetically modify or differentiate the MSC population in a particular fashion.

“Cell replacement therapy” as used herein, refers to the transplantation of undifferentiated or differentiated mesenchymal stem cells into a patient for the treatment of a disease. For the treatment of brain diseases, the cells are typically transplanted into the brain of the subject.

Cell replacement therapy may be performed using a variety of transplantation approaches, the nature of which depends on the site of implantation.

According to a particular embodiment, the cells are administered intranasally.

The term or phrase “transplantation”, “cell replacement” or “grafting” are used interchangeably herein and refer to the introduction of the cells of the present invention to target tissue. As mentioned, the cells can be derived from the recipient or from an allogeneic, semi-allogeneic or xenogeneic donor.

The cells can be injected systemically into the circulation, administered intrathecally or grafted into the central nervous system, the spinal cord or into the ventricular cavities or subdurally onto the surface of a host brain. Conditions for successful transplantation include: (i) viability of the implant; (ii) retention of the graft at the site of transplantation; and (iii) minimum amount of pathological reaction at the site of transplantation. Methods for transplanting various nerve tissues, for example embryonic brain tissue, into host brains have been described in: “Neural grafting in the mammalian CNS”, Bjorklund and Stenevi, eds. (1985); Freed et al., 2001; Olanow et al., 2003). These procedures include intraparenchymal transplantation, i.e. within the host brain (as compared to outside the brain or extraparenchymal transplantation) achieved by injection or deposition of tissue within the brain parenchyma at the time of transplantation.

Intraparenchymal transplantation can be performed using two approaches: (i) injection of cells into the host brain parenchyma or (ii) preparing a cavity by surgical means to expose the host brain parenchyma and then depositing the graft into the cavity. Both methods provide parenchymal deposition between the graft and host brain tissue at the time of grafting, and both facilitate anatomical integration between the graft and host brain tissue. This is of importance if it is required that the graft becomes an integral part of the host brain and survives for the life of the host.

Alternatively, the graft may be placed in a ventricle, e.g. a cerebral ventricle or subdurally, i.e. on the surface of the host brain where it is separated from the host brain parenchyma by the intervening pia mater or arachnoid and pia mater. Grafting to the ventricle may be accomplished by injection of the donor cells or by growing the cells in a substrate such as 3% collagen to form a plug of solid tissue which may then be implanted into the ventricle to prevent dislocation of the graft. For subdural grafting, the cells may be injected around the surface of the brain after making a slit in the dura. Injections into selected regions of the host brain may be made by drilling a hole and piercing the dura to permit the needle of a microsyringe to be inserted. The microsyringe is preferably mounted in a stereotaxic frame and three dimensional stereotaxic coordinates are selected for placing the needle into the desired location of the brain or spinal cord. The cells may also be introduced into the putamen, nucleus basalis, hippocampus cortex, striatum, substantia nigra or caudate regions of the brain, as well as the spinal cord.

The cells may also be transplanted to a healthy region of the tissue. In some cases the exact location of the damaged tissue area may be unknown and the cells may be inadvertently transplanted to a healthy region. In other cases, it may be preferable to administer the cells to a healthy region, thereby avoiding any further damage to that region. Whatever the case, following transplantation, the cells preferably migrate to the damaged area.

For transplanting, the cell suspension is drawn up into the syringe and administered to anesthetized transplantation recipients. Multiple injections may be made using this procedure.

The cellular suspension procedure thus permits grafting of the cells to any predetermined site in the brain or spinal cord, is relatively non-traumatic, allows multiple grafting simultaneously in several different sites or the same site using the same cell suspension, and permits mixtures of cells from different anatomical regions. Multiple grafts may consist of a mixture of cell types, and/or a mixture of transgenes inserted into the cells. Preferably from approximately 10⁴ to approximately 10⁹ cells are introduced per graft. Cells can be administered concomitantly to different locations such as combined administration intrathecally and intravenously to maximize the chance of targeting into affected areas.

For transplantation into cavities, which may be preferred for spinal cord grafting, tissue is removed from regions close to the external surface of the central nerve system (CNS) to form a transplantation cavity, for example as described by Stenevi et al. (Brain Res. 114:1-20, 1976), by removing bone overlying the brain and stopping bleeding with a material such a gelfoam. Suction may be used to create the cavity. The graft is then placed in the cavity. More than one transplant may be placed in the same cavity using injection of cells or solid tissue implants. Preferably, the site of implantation is dictated by the CNS disorder being treated. Demyelinated MS lesions are distributed across multiple locations throughout the CNS, such that effective treatment of MS may rely more on the migratory ability of the cells to the appropriate target sites.

As mentioned, the method of this aspect of the present invention includes ex vivo differentiating a population of mesenchymal stem cells originating from the subject towards a first lineage-specific cell, the first lineage-specific cell being associated with a brain disorder

The term “lineage specific cell” refers to a cell that is no longer mutipotent but is committed towards a particular cell type. The lineage specific cell may be a progenitor cell (e.g. a neural stem cell) or a fully differentiated cell (e.g. a motor neuron).

Examples of lineage specific cells contemplated by the present invention include motor neurons, neural stem cells, skeletal muscle cells, dopamine secreting neurons, cholinergic neurons, astrocytes oligodendrocytes and progenitors thereof.

Selection of particular lineage specific cells towards which the MSCs are differentiated is dependent on the required qualification.

Thus, for example, if the MSCs are being qualified as being suitable for the autologous treatment of a brain disease, the lineage specific cell is selected according to the brain disease for which the MSCs are intended to treat.

Thus, for example in the case where the disease is ALS, contemplated lineage specific cells include astrocytes, skeletal muscle cells and motor neuron progenitors or fully differentiated cells.

Thus, for example in the case where the disease is Parkinson's, contemplated lineage specific cells include dopaminergic neurons, astrocytes and oligodendrocyte progenitors or fully differentiated cells.

Thus, for example in the case where the disease is Alzheimer's disease, contemplated lineage specific cells include neurons and astrocyte progenitors or fully differentiated cells.

Thus, for example in the case where the disease is Rett's syndrome, contemplated lineage specific cells include neurons and astrocyte progenitors or fully differentiated cells.

Methods of differentiating MSCs towards the above mentioned lineage specific cells are known in the art and include genetic modification and/or culture in a differentiating medium.

Genetic Modification

MSCs may be genetically modified to express a protein which induces the differentiation thereof. Alternatively, MSCs may be genetically modified to express a polynucleotide agent e.g. an siRNA or miRNA that down-regulates expression of a particular protein or polynucleotide agent in order to induce differentiation.

To express such agents in mesencyhymal stem cells, a polynucleotide sequence encoding the agent is preferably ligated into a nucleic acid construct suitable for mesenchymal stem cell expression. Such a nucleic acid construct includes a promoter sequence for directing transcription of the polynucleotide sequence in the cell in a constitutive or inducible manner.

Constitutive promoters suitable for use with some embodiments of the invention are promoter sequences which are active under most environmental conditions and most types of cells such as the cytomegalovirus (CMV) and Rous sarcoma virus (RSV). Inducible promoters suitable for use with some embodiments of the invention include for example tetracycline-inducible promoter (Zabala M, et al., Cancer Res. 2004, 64(8): 2799-804).

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed—i.e. mesenchymal stem cells.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

According to one embodiment, a lentiviral vector is used to transfect the mesenchymal stem cells.

Various methods can be used to introduce the expression vector of some embodiments of the invention into mesenchymal stem cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Other vectors can be used that are non-viral, such as cationic lipids, polylysine, and dendrimers. Nanoparticles are also contemplated.

Other modes of transfection that do not involved integration include the use of minicircle DNA vectors or the use of PiggyBac transposon that allows the transfection of genes that can be later removed from the genome.

According to a particular embodiment, the mesenchymal stem cells are genetically modified to express a miRNA (or group of miRNAs) in order to induce differentiation. Alternatively, or additionally, the mesenchymal stem cells are genetically modified to express a polynucleotide agent that down regulates a miRNA (or group of miRNAs) in order to induce differentiation.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms and have been shown to play a role in development, homeostasis, and disease etiology.

Below is a brief description of the mechanism of miRNA activity.

Genes coding for miRNAs are transcribed leading to production of an miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nt precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GTP and the export receptor exportin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually become incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al (2005, Nat Genet. 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to down regulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

The term “microRNA mimic” refers to synthetic non-coding RNAs that are capable of entering the RNAi pathway and regulating gene expression. miRNA mimics imitate the function of endogenous microRNAs (miRNAs) and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O,4′-C-ethylene-bridged nucleic acids (ENA)). Other modifications are described herein below. For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

It will be appreciated from the description provided herein above that genetically modifying mesenchymal stem cells to express miRNAs may be affected in a number of ways:
1. Transiently transfecting the mesenchymal stem cells with the mature miRNA (or modified form thereof, as described herein below). The miRNAs designed according to the teachings of the present invention can be generated according to any oligonucleotide synthesis method known in the art, including both enzymatic syntheses and solid-phase syntheses. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Md.; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.
2. Stably, or transiently transfecting the mesenchymal stem cells with an expression vector which encodes the mature miRNA.
3. Stably, or transiently transfecting the mesenchymal stem cells with an expression vector which encodes the pre-miRNA. The pre-miRNA sequence may comprise from 45-90, 60-80 or 60-70 nucleotides. The sequence of the pre-miRNA may comprise a miRNA and a miRNA* as set forth herein. The sequence of the pre-miRNA may also be that of a pri-miRNA excluding from 0-160 nucleotides from the 5′ and 3′ ends of the pri-miRNA.
4. Stably, or transiently transfecting the mesenchymal stem cells with an expression vector which encodes the pri-miRNA. The pri-miRNA sequence may comprise from 45-30,000, 50-25,000, 100-20,000, 1,000-1,500 or 80-100 nucleotides. The sequence of the pri-miRNA may comprise a pre-miRNA, miRNA and miRNA*, as set forth herein, and variants thereof. Preparation of miRNAs mimics can be effected by chemical synthesis methods or by recombinant methods.

Culture in a Differentiation Medium:

Contacting MSCs with differentiation agents can be performed under any in vitro conditions including for example, adding the agent to cells derived from a subject such that the agent is in direct contact with the cells. According to some embodiments of the invention, the cells of the subject are incubated with the differentiating agent. The conditions used for incubating the cells are selected for a time period/concentration of cells/concentration of agent/ratio between cells and agent and the like which enable the agent to induce cellular changes that promote differentiation, such as changes in transcription and/or translation rate of specific genes.

Typically, the medium comprises growth factors and/or cytokines which promote the differentiation of a particular lineage specific cell.

Examples of differentiating media include G5, neurobasal medium, DMEM or DMEM/F12, OptiMEM™ or any other medium that supports growth of the lineage specific cell.

Examples of differentiating agents include, but are not limited to, human neuregulin 1-β1, N2 supplement, IBMX, cAMP, neurotrophic factors (e.g. BDNF, CNTF, GDNF, NTN, NT3 or LIF), hormones, growth factors (e.g. GGF2, TGF-β3, TGF-α, FGF, including FGF-8, FGF4 and bFGF, EGF, platelet derived growth factor (PDGF)), vitamins, hormones e.g., insulin, progesterone and other factors such as sonic hedgehog, bone morphogenetic proteins, forskolin, retinoic acid, ascorbic acid, putrescin, selenium and transferrin.

Below is a summary of exemplary differentiation protocols that may be used to generate the lineage specific cells. It will be appreciated that many more protocols for MSC differentiation are known in the art and the present invention contemplates all such protocols.

Astrocyte-Like Cells:

The present inventors contemplate genetic modification of MSCs in order to induce astrocytic differentiation thereof by up or down regulation of a particular set of miRNAs.

Thus, according to one embodiment, differentiation towards an astrocytic phenotype may be effected by up-regulating a level of at least one exogenous miRNA being selected from the group consisting of miR-18, miR-17-5p, miR-141, miR-302b, miR-20b, miR-101, miR-126, miR-146a, miR-146b, miR-3a, miR-26, miR-29, miR-132, miR-92ap, miR-21, miR-26a, miR-18a, miR-124, miR-99a, miR-30c, miR-301a, miR-145-50, miR-143-3p, miR-373, miR-20b, miR-29c, miR-29b, miR-143, let-7g, let-7a, let-7b, miR-98, miR-30a*, miR-17, miR-1, miR-192, miR-155, miR-516-ap, miR-31, miR-181a, miR-181b, miR-181c, miR-34-c, miR-34b*, miR-103a, miR-210, miR-16, miR-30a, miR-31, miR-222, miR-17, miR-17*, miR-200b, miR-200c, miR-128, miR-503, miR-424, miR-195, miR-1256, miR-203a, miR-199, miR-93, miR-98, miR-125-a, miR-133a, miR-133b, miR-126, miR-194, miR-346, miR-15b, miR-338-3p, miR-373, miR-205, miR-210, miR-125, miR-1226, miR-708, miR-449, miR-422, miR-340, miR-605, miR-522, miR-663, miR-130a, miR-130b, miR-942, miR-572, miR-520, miR-639, miR-654, miR-519, mir-202, mir-767-5p, mir-29a, mir-29b, mir-29c, let-7a, let-7b, let-7c, let-7d, let-7e, let-7f, let-7g, let-7i, mir-4458, mir-4500, mir-98, mir-148a, mir-148b, mir-152, mir-4658, mir-3662, mir-25, mir-32, mir-363, mir-367, mir-92a, mir-92b, mir-520d-5p, mir-524-5p, mir-4724-3p, mir-1294, mir-143, mir-4770, mir-3659, mir-145, mir-3163, mir-181a, mir-181b, mir-181c, mir-181d, mir-4262, mir-4279, mir-144, mir-642b, mir-4742-3p, mir-3177-5p, mir-656, mir-3121-3p, mir-106a, mir-106b, mir-17, mir-20a, mir-20b, mir-519d, mir-93, mir-1297, mir-26a, mir-26b, mir-4465, mir-326, mir-330-5p, mir-3927, mir-2113, mir-372, mir-373, mir-520a-3p, mir-520b, mir-520c-3p, mir-520d-3p, mir-520e, mir-199a-3p, mir-199b-3p, mir-3129-5p in mesenchymal stem cells (MSCs).

According to another embodiment, differentiation towards an astrocytic phenotype may be effected by down-regulating an expression of at least one miRNA, the miRNA being selected from the group consisting of mi-R-193b, mi-R-221, mi-R-135a, mi-R-149, mi-R-222, mi-R-199a, mi-R-302a, mi-R-302c, mi-R-302d, mi-R-369-3p, mi-R-370, mi-R-let7a, mi-R-let7b, mi-R-10b, mi-R-23a, mi-R-23b and mi-R-32, miR-204, miR-224, miR-616, miR-122, miR-299, miR-100, miR-138, miR-140, miR-375, miR-217, miR-302, miR-372, miR-96, miR-127-3p, miR-449, miR-135b, miR-101, miR-326, miR-324, miR-335, miR-14, miR-16, mir-410, mir-3163, mir-148a, mir-148b, mir-152, mir-3121-3p, mir-495, mir-203 and mir-4680-3p wherein the down-regulating is effected by up-regulating a level of at least one polynucleotide agent that hybridizes and inhibits a function of the at least one.

Particular combinations are also contemplated by the present inventors including, but not limited to up-regulation of each of miR-20b, the miR-101 and the miR-146a and up-regulation of each of miR-9 and exogenous miR-20b in the MSCs.

Up-regulating a level of exogenous miR-9, exogenous miR-146 and exogenous miR-101 in a population of MSCs and down-regulating an expression of miR-10b and miR-302 in the population of MSCs is another combination contemplated by the present inventors.

Methods of differentiating MSCs towards an astrocytic lineage are disclosed in Kopen, G. C., et al., Proc Natl Acad USA. 96(19):10711-6, 1999; Sanchez-Ramos, et al. Exp Neurol. 164(2):247-56. 2000; Woodbury, D., J Neurosci Res. 61(4):364-70, 2000; Woodbury, D., et al., J Neurosci Res. 69(6):908-17, 2002; Black, I. B., Woodbury, D. Blood Cells Mol Dis. 27(3):632-6, 2001; Kohyama, J., et al. Differentiation. 68(4-5):235-44, 2001; Levy, Y. S. J Mol Neurosci. 21(2):121-32, 2003; US Patent Application No. 20090010895; US Patent Application No. 20120009673, U.S. Pat. No. 6,528,24; Reyes and Verfatile (Ann N.Y. Acad. Sci. 938:231-235, 2001) and Jiang et al. (Nature 418:47-49, 2002), the contents of which are incorporated herein by reference.

Neural Stem Cells:

The present inventors contemplate genetic modification of MSCs in order to induce neural stem cell differentiation thereof by up or down regulation of a particular set of miRNAs.

Thus, according to one embodiment, differentiation towards an neural stem cell phenotype may be effected by up-regulating a level of at least one exogenous miRNA selected from the group consisting of miR302b, miR-371, miR-134, miR-219, miR-154, miR-155, miR-32, miR-33, miR-126, miR-127, miR-132, miR-137, miR-572, miR-935a, miR-891a, miR-1202, miR-1275, let-7c, miR-665, miR-4258, miR-361-3p, miR-374a-star, miR-892b miR-361-5p, miR-181a, miR-16, miR-636, miR-4284, miR-1208, miR-1274b, miR-30c-2-star, miR-501-3p, hsa-miR-92a, miR-378b, miR-1287, miR-425-star, miR-324-5p, miR-3178, miR-219-1-3p, miR-197, miR-181b, miR-500-star, miR-106b, miR-502-3p, miR-30c, miR-1275, miR-422a, miR-93, miR-181d, miR-1307, miR-1301, miR-99a, miR-505-star, miR-1202, miR-12, miR-532-5p, miR-195, miR-532-3p, miR-106a, miR-17, miR-1271, miR-769-3p, miR-15b, miR-324-3p, miR-20a, miR-501-5p, miR-330-3p, miR-874, miR-500, miR-25, miR-769-5p, miR-125b-2-star, miR-130b, miR-504, miR-181a-2-star, miR-885-3p, miR-1246, miR-92b, miR-362-5p, miR-572, miR-4270, miR-378c, miR-93-star, miR-149, miR-363, miR-18a, miR-891a, miR-346, miR-497, miR-378, miR-1231, miR-139-5p, miR-3180-3p, miR-935 and miR-20b in mesenchymal stem cells (MSCs).

According to another embodiment, differentiation towards a neural stem cell phenotype may be effected by down-regulating an expression of at least one miRNA selected from the group consisting of miR-10b, miR-142-3p, miR-131a, miR-125b, miR-153, miR-181a, miR-409-5p, miR-193a-5p, miR-4317, miR-4288, miR-145, miR-143, miR-214, miR-199a-3p, miR-199a-5p, miR-199b-3p, miR-138, miR-31, miR-21, miR-193a-5p, miR-224-star, miR-196a, miR-487b, miR-409-5p, miR-193b-star, miR-379, miR-21-star, miR-27a-star, miR-27a, miR-4317, miR-193b, miR-27b, miR-22, 574-3p, miR-4288, miR-23a, miR-221-star, miR-2113, let-7i, miR-24, miR-23b, miR-299-3p, miR-518c-star, miR-221, miR-431-star, miR-523, miR-4313, miR-559, miR-614, miR-653, miR-2278, miR-768-5p, miR-154-star, miR-302a-star, miR-3199 and miR-3137 in mesenchymal stem cells by up-regulating a level of at least one polynucleotide agent that hybridizes and inhibits a function of the at least one miRNA.

According to still another embodiment, differentiation towards a neural stem cell phenotype may be effected by up-regulating a level of exogenous miR-124 in mesenchymal stem cells (MSCs) and down-regulating a level of miR-let-7 in the MSCs.

Another method of generating neural stem cells from MSCs is by the down-regulation of an amount and/or activity of Related to testis-specific, vespid and pathogenesis protein 1 (RTVP-1).

Additional methods of generating neural stem cells are known in the art and the present invention contemplates all such methods.

Motor Neurons:

The present inventors contemplate genetic modification of MSCs via the generation of neural stem cells in order to induce motor neuron differentiation thereof by up or down regulation of a particular set of miRNAs.

Thus, according to one embodiment, differentiation towards motor neuron phenotype may be effected by up-regulating a level of at least one exogenous miRNA selected from the group consisting of miR-368, miR-302b, miR-365-3p, miR-365-5p, miR-Let-7a, miR-Let-7b, miR-218, miR-134, miR-124, miR-125a, miR-9, miR-154, miR-20a, miR-130a in neural stem cells (NSCs).

According to still another embodiment, differentiation towards a motor neuron phenotype may be effected by down-regulating an expression of at least one miRNA selected from the group consisting of miR-372, miR-373, miR-141, miR-199a, miR-32, miR-33, miR-221 and miR-223 by up-regulating a level of at least one polynucleotide agent that hybridizes and inhibits a function of the at least one miRNA in NSCs.

Particular combination of miRNAs are also contemplated by the present inventors including for example up-regulating each of miR Let-7a, miR-124, miR-368 and miR-154 or up-regulating each of miR-125a, miR-9, miR-130a or up-regulating each of miR-218, miR-134 and miR-20a in neural stem cells.

Additionally, or alternatively the present inventors further contemplate down-regulating each of miR-141, miR-32, miR-33, miR-221, miR-223 and miR-373 in neural stem cells.

In addition, generation of motor neurons may be effected by up-regulating a level of the following exogenous miRNAs in mesenchymal stem cells:

miR-368, miR-365, miR-500, miR-648, miR-491, miR-218, miR-155, miR-192, let-7b, miR-16, miR-210, miR-197, miR-21, miR-373, miR-27a, miR-122, miR-17, miR-494, miR-449, miR-503, miR-30a, miR-196a, miR-122, miR-7, miR-151-5p, miR-16, miR-22, miR-31, miR-424, miR-1, miR-29c, miR-942, miR-100, miR-520, miR-663a, miR-562, miR-449a, miR-449b-5p, miR-520b, miR-451, miR-532-59, miR-605, miR-504, miR-503, miR-155, miR-34a, miR-16, miR-7b, miR-103, miR-124, miR-1385p, miR-16, miR-330, miR-520, miR-608, miR-708, miR-107, miR-137, miR-132, miR-145, miR-204, miR-125b, miR-224, miR-30a, miR-375, miR-101, miR-106b, miR-128, miR-129-5p, miR-153, miR-203, miR-214, miR-338-3p, miR-346, miR-98, miR-107, miR-141, miR-217, miR-424, miR-449, miR-7, miR-9, miR-93, miR-99a, miR-100, miR-1228, miR-183, miR-185, miR-190, miR-522, miR-650, miR-675, miR-342-3p, miR-31.

In addition, generation of motor neurons may be effected by up-regulating a level of at least one polynucleotide agent that hybridizes and inhibits a function of least one of the following miRNA in the MSCs:

miR-199a, miR-372, miR-373, miR-942, miR-2113, miR-301a-3p, miR-302c, miR-30b-5p, miR-30c, miR-326, miR-328, miR-331-3p, miR-340, miR-345, miR-361-5p, miR-363, miR-365a-3p, miR-371a-3p, miR-373-3p, miR-374a, miR-423-3p, miR-449b-5p, miR-451a, miR-494, miR-504, miR-515-3p, miR-516a-3p, miR-519e, miR-520a-3p, miR-520c-3p, miR-520g, miR-532-5p, miR-559, miR-562, miR-572, miR-590-5p, miR-605, miR-608, miR-626, miR-639, miR-654-3p, miR-657, miR-661, miR-708-5p, miR-942, miR-96, miR-99arno and miR-194.

Roy et al., 2005 [Exp Neurol. 2005; 196:224-234]; Zhang et al., 2006 [Stem Cells. 2006; 24:434-442]; Bohl et al., 2008 [Stem Cells. 2008; 26:2564-2575]; and Dimos et al., 2008 [Science. 2008; 321:1218-1221] the contents of which are incorporated by reference teach genetic modification of a variety of different stem cells to induce differentiation into motor neurons. One of skill in the art would know how to adapt such protocols for mesenchymal stem cell therapy.

Renoncourt et al., 1998 [Mech Dev. 1998; 79:185-197]; Wichterle et al., 2002 Cell 2002; 110:385-397; Barberi et al., 2003 [Nat Biotechnol. 2003; 21:1200-1207]; Li et al., 2005 [Nat Biotechnol. 2005; 23:215-221]; Shin et al., 2005 [Stem Cells Dev. 2005; 14:266-269], Soundararajan et al., 2006 [J Neurosci. 2006; 26:3256-3268], Lim et al., 2006 [Curr Neurovasc Res. 2006; 3:281-288]; Deshpande et al., 2006 [Ann Neurol. 2006; 60:32-44]; Lee et al., 2007 [Stem Cells. 2007; 25:1931-1939]; Li et al., 2008 [Stem Cells. 2008; 26:886-893]; Wu et al., 2002 [Nat Neurosci. 2002; 5:1271-1278]; Gao et al., 2005 [Neuroscience. 2005; 131:257-262]; MacDonald et al., 2003 [J Neurosurg. 2003; 98:1094-1103]; Goncalves et al., 2005 [Dev Biol. 2005; 278:60-70]; Corti et al., 2006 [Hum Mol Genet. 2006; 15:167-187]; Corti et al., 2007 [Brain. 2007; 130:1289-1305] the contents of which are incorporated by reference teach culturing in differentiation medium of a variety of different stem cells to induce differentiation into motor neurons. One of skill in the art would know how to adapt such protocols for mesenchymal stem cell therapy.

Skeletal Muscle Cells

Contemplated protocols for differentiation of MSCs towards skeletal muscles include those disclosed by Taylor S M, Jones P A. J Cell Physiol 1982; 111:187-94; Lee J H, Kosinski P A, Kemp D M. Exp Cell Res 2005; 307:174-82, the contents of which are incorporated by reference.

Neurons

International Application No. WO 2010/144698 to the present inventors, incorporated herein by reference, teaches genetic modification of MSCs in order to induce neuronal differentiation thereof. This application teaches the up or down regulation of a particular set of miRNAs in order to promote differentiation.

U.S. patent application 20050265983 teaches differentiation of MSCs towards dopamine secreting cells.

MSCs have also been shown to differentiate into neuron-like cells demonstrating neuronal markers (Azizi et al., 1998, Proc Natl Acad Sci USA 95:3908-3913; Deng et al., 2001, Biochem Biophys Res Commun 282:148-152; Kopen et al., Proc Natl Acad USA 96:10711-10716 1999; Levy et al., 2003 J Mol. Neurosci. 21:127-138; Sanchez-Ramos et al., 2000 Exp Neurol 164:247-256; Schwarz et al., 1999 Hum Gene Ther 10:2539-2549; Woodbury et al., 2000, J Neurosci Res 61:364-370) and some electro-physiological functions (Kohyama et al., 2001, Differentiation 68: 235-244). The cells have also been shown to express dopaminergic markers and also to secrete dopamine following depolarization (Levy et al., 2004, J Mol. Neurosci. 24:353-386).

Oligodendrocytes:

Contemplated protocols include those disclosed by Liu et al [Dev Biol. 302:683-693, 2007] and U.S. Patent Application No. 20100021434 teaches oligodendrocytic differentiation of bone marrow derived mesenchymal cells by incubation in N2 supplement and fibroblast growth factor (FGF), the contents of both are incorporated herein by reference.

IL2011/000660 to the present inventors, incorporated herein by reference, teaches genetic modification of MSCs in order to induce neuronal differentiation thereof. This application teaches the up or down regulation of a particular set of miRNAs in order to promote differentiation.

In order to establish whether a mesenchymal stem cell population is suitable for autologous transplantation, the lineage specific cell (which is differentiated from the MSCs of the patient) is contacted with another lineage specific cell associated with the disease and the effect thereupon is analyzed.

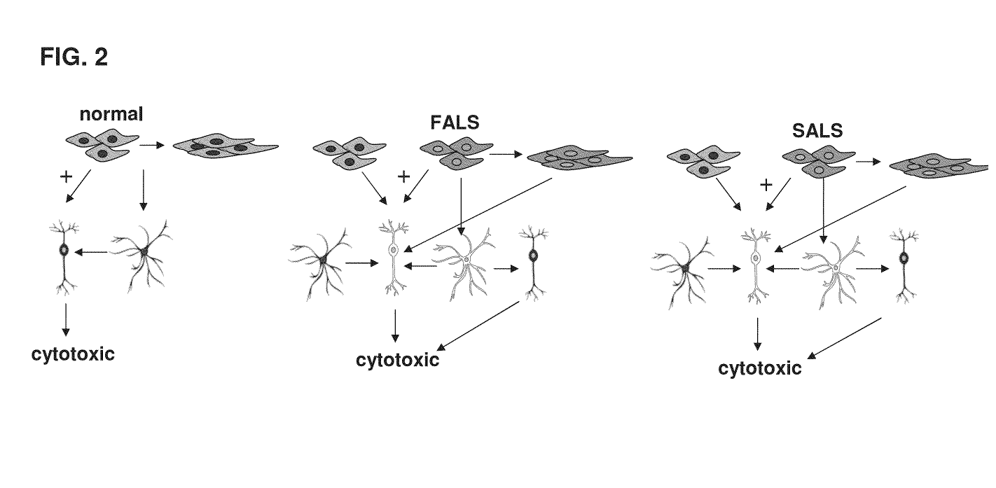

Amyotrophic Lateral Sclerosis (ALS)—See FIG. 2:

Amyotrophic lateral sclerosis (ALS) is a fatal neurodegenerative disease of unknown cause affecting the upper and lower motor neurons of patients. Approximately 5,600 people in the U.S. are diagnosed with ALS each year. The incidence of ALS is two per 100,000 people, and it is estimated that as many as 30,000 Americans may have the disease at any given time. 80% of patients die within five years following diagnosis, where normally death is due to respiratory failure. To this day, there is no effective treatment for ALS; the only drug currently approved by the FDA is a NMDA receptor antagonist (riluzole) that increases the rate of survival by 6 months.

In order to establish whether a mesenchymal stem cell population is suitable for autologous transplantation for the treatment of ALS, astrocytes and/or skeletal muscle cells which have been differentiated from the patient may be contacted (e.g. co-cultured) with motor neurons. The motor neurons may be primary motor neurons or cell lines of motor neurons. According to a particular embodiment, the motor neurons are differentiated from the mesenchymal stem cell population from which the astrocytes have been differentiated.

Preferably, the cytotoxic effect of the lineage specific cells on the motor neurons is analyzed.

Methods of monitoring cellular changes induced by the drugs are known in the art and include for example, the MTT test which is based on the selective ability of living cells to reduce the yellow salt MTT (3-(4,5-dimethylthiazolyl-2)-2,5-diphenyltetrazolium bromide) (Sigma, Aldrich St Louis, Mo., USA) to a purple-blue insoluble formazan precipitate; the BrDu assay [Cell Proliferation ELISA BrdU colorimetric kit (Roche, Mannheim, Germany]; the TUNEL assay [Roche, Mannheim, Germany]; the Annexin V assay [ApoAlert® Annexin V Apoptosis Kit (Clontech Laboratories, Inc., CA, USA)]; the Senescence associated-β-galactosidase assay, as well as various RNA and protein detection methods (which detect level of expression and/or activity).

If the lineage specific cells derived from the ALS patient are more cytotoxic to motor neurons than the identical lineage specific cells derived from a control subject, the MSC population from which they are derived may be deemed as unsuitable for autologous cell transplantation for the treatment of ALS.

According to one embodiment, when the autologous MSC population is greater than 10% more cytotoxic than the control MSC population, the patient cell population is deemed unsuitable for autologous cell population for the treatment of ALS.

According to one embodiment, when the autologous MSC population is greater than 20% more cytotoxic than the control MSC population, the patient cell population is deemed unsuitable for autologous cell population for the treatment of ALS.

According to one embodiment, when the autologous MSC population is greater than 30% more cytotoxic than the control MSC population, the patient cell population is deemed unsuitable for autologous cell population for the treatment of ALS.

According to one embodiment, when the autologous MSC population is greater than 40% more cytotoxic than the control MSC population, the patient cell population is deemed unsuitable for autologous cell population for the treatment of ALS.

According to one embodiment, when the autologous MSC population is greater than 50% more cytotoxic than the control MSC population, the patient cell population is deemed unsuitable for autologous cell population for the treatment of ALS.

The control MSC population is typically derived from a subject who does not have ALS, preferably a healthy subject. Age and sex matching of MSC populations is also contemplated.

Preferably, the control MSC population is differentiated towards the lineage-specific cell concurrently with the differentiation of the patient MSC population so as to ensure that the two cell populations undergo identical experimental conditions, such as temperature and time etc.

In addition to cell death, the motor neurons may be examined for susceptibility to oxidative stress, proteasome inhibition, mitochondria function and ER stress response following co-culture with the lineage specific cells differentiated from the patient MSC populations.

As well as the co-culture assay described herein above, the present invention further contemplates analyzing for a genetic aberration of the MSCs originating from the subject. If the genetic aberration is detected, then the MSC population is not selected as a source for cell therapy. Thus, for example the present invention contemplates analyzing for the existence of a mutation in the superoxide dismutase (SOD-1) gene.

The SOD-1 gene is localized to chromosome 21q22.1. SOD-1 sequences are disclosed in PCT publication WO 94/19493. The nucleic acid sequence of human SOD-1 gene can be found at Genbank accession no. NM. 000454.

Other genetic aberrations associated with ALS include those present in Alsin, a potential guanine-nucleotide exchange factor (GEF) responsible for the juvenile recessive form of ALS. Another is ALS4 that encodes for a DNA/RNA helicase domain containing protein called Senataxin identified to be linked to the autosomal dominant form of juvenile ALS. Most recently, a mutation in the vesicle associated membrane protein/synaptobrevin associated membrane protein B (VAPB) in a new locus called ALS8, was reported to be associated with an atypical form of ALS.

According to another embodiment, the genetic aberration is in the TARDBP gene.

Methods of analyzing for a genetic aberration are known in the art and may be effected on the protein or polynucleotide level.

Exemplary methods for analyzing genetic aberrations include Chromosomal and DNA staining methods (e.g. FISH analysis, PRINS analysis, High-resolution multicolor banding (MCB) on interphase chromosomes and quantitative FISH (Q-FISH.

The sequence alteration (or SNP) of some embodiments of the invention can be identified using a variety of additional methods. One option is to determine the entire gene sequence of a PCR reaction product. Alternatively, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

Additional exemplary techniques include restriction fragment length polymorphism (RFLP), sequencing analysis, microsequencing, mismatch detection assays based on polymerases and ligases, ligase/Polymerase-mediated Genetic Bit Analysis™ hybridization Assay methods including hybridization to oligonucleotide arrays, Integrated Systems, allele specific oligonucleotide (ASO), Denaturing/Temperature Gradient Gel Electrophoresis (DGGE/TGGE), single-strand conformation polymorphism (SSCP); Dideoxy fingerprinting (ddF), Pyrosequencing™ analysis (Pyrosequencing, Inc. Westborough, Mass., USA); Acycloprime™ analysis (Perkin Elmer, Boston, Mass., USA) and reverse dot blot:

Sequence alterations can also be determined at the protein level. While chromatography and electrophoretic methods are preferably used to detect large variations in SOD-1 molecular weight, immunodetection assays such as ELISA and western blot analysis, immunohistochemistry and the like, which may be effected using antibodies specific to SPD-1 sequence alterations are preferably used to detect point mutations and subtle changes in molecular weight.

Thus, the present invention according to some embodiments thereof also envisages the use of serum immunoglobulins, polyclonal antibodies or fragments thereof, (i.e., immunoreactive derivatives thereof), or monoclonal antibodies or fragments thereof for the detection of genetic aberrations. Monoclonal antibodies or purified fragments of the monoclonal antibodies having at least a portion of an antigen-binding region, including the fragments described herein below, chimeric or humanized antibodies and complementarily determining regions (CDR).

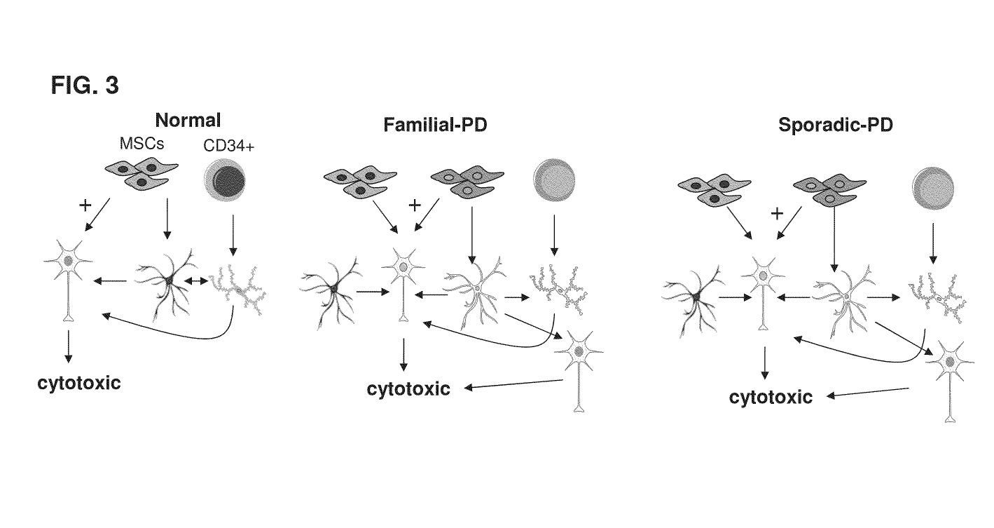

Parkinson's Disease—See FIG. 3:

Parkinson's disease (PD) is a progressive neurodegenerative disease that is characterized by rigidity, bradykinesia and resting tremor. The pathological hallmarks of this disease are the selective loss of dopaminergic neurons in the substantia nigra pars compacta (SNc) in the midbrain. Most of the cases of PD occur sporadically with yet unknown causes and pathogenesis. Potential mechanisms include mitochondrial dysfunction, oxidative stress, endoplasmic reticulum stress, failure of the ubiquitin-proteasome system, some environmental factors, and genetic predisposition.

Astrocytes have been recently reported to play a role in the progression and in initiating the early neuronal dysfunction and α-synuclein accumulation in astrocytes lead to the cell death of specific neuronal populations in restricted brain regions causing the clinical symptoms of PD. Recent studies indicate that accumulation of a-synuclein in axon-ensheathing oligodendrocytes can also induce neurodegeneration of the associated neurons. Finally, microglia cells have been shown to undergo activated by astrocytes in early staged of PD, which then exert toxic effects on the dopaminergic neurons. Thus, similar to other neurodegenerative disorders the pathogenesis of PD is mediated by pathological cell-cell interactions.

In order to establish whether a mesenchymal stem cell population is suitable for autologous transplantation for the treatment of Parkinson's disease, astrocytes which have been differentiated from the patient may be contacted (e.g. co-cultured) with dopaminergic neurons. The dopaminergic neurons may be primary dopaminergic neurons or cell lines of dopaminergic neurons. According to a particular embodiment, the dopaminergic neurons are differentiated from the mesenchymal stem cell population from which the astrocytes have been differentiated.

Preferably, the cytotoxic effect of the lineage specific cells on the dopaminergic neurons is analyzed.

According to a particular embodiment, microglia cells differentiated from CD34+ cells (i.e. hematopoietic progenitors obtained from the bone marrow or whole blood derived from the patient are co-cultured with the dopaminergic neurons either in the presence or absence of the mesenchymal cell derived astrocytic cells. The cells may be presorted so as to comprise an enriched population of CD34+ cells.

Methods of differentiating CD34+ cells or whole bone marrow into a microglia phenotype are known in the art and include for example culturing for 10-15 days (e.g. 11 days) in culturing medium (e.g. DMEM/10% FCS). Non-adherent cells are further cultured for a period of 5-10 days (e.g. 6 days). The cells are then aspirated and transferred to a new culturing dish and cultured in medium (e.g. DMEM/10% FCS) supplemented with 50% astrocyte conditioned medium (prepared from primary human astrocytes cultured in medium (e.g. DMEM for about 24 hr) and 20 ng/ml GM-CSF. The cells then express microglia markers and function similar to microglia cells.

Methods of analyzing cytotoxicity have been described herein above.

If the lineage specific cells derived from the patient are more cytotoxic to dopaminergic neurons than the identical lineage specific cells derived from a control subject, the MSC population from which they are derived may be deemed as unsuitable for autologous cell transplantation for the treatment of PD.

According to one embodiment, when the autologous MSC population is greater than 10% more cytotoxic than the control MSC population, the patient cell population is deemed unsuitable for autologous cell population for the treatment of PD.

According to one embodiment, when the autologous MSC population is greater than 20% more cytotoxic than the control MSC population, the patient cell population is deemed unsuitable for autologous cell population for the treatment of PD.

According to one embodiment, when the autologous MSC population is greater than 30% more cytotoxic than the control MSC population, the patient cell population is deemed unsuitable for autologous cell population for the treatment of PD.

According to one embodiment, when the autologous MSC population is greater than 40% more cytotoxic than the control MSC population, the patient cell population is deemed unsuitable for autologous cell population for the treatment of PD.

According to one embodiment, when the autologous MSC population is greater than 50% more cytotoxic than the control MSC population, the patient cell population is deemed unsuitable for autologous cell population for the treatment of PD.

The control MSC population is typically derived from a subject who does not have Parkinson's disease, preferably a healthy subject. Age and sex matching of MSC populations is also contemplated.

In addition to cell death, the dopaminergic neurons may be examined for susceptibility to oxidative stress, proteasome inhibition, appearance of insoluble α-synuclein proteins, mitochondria function and ER stress response following co-culture with the lineage specific cells differentiated from the patient MSC populations.

As well as the co-culture assay described herein above, the present invention further contemplates analyzing for a genetic aberration of the MSCs originating from the subject. If the genetic aberration is detected, then the MSC population is not selected as a source for cell therapy. Thus, for example the present invention contemplates analyzing for the existence of a mutation in alpha-synuclein gene (e.g. alanine 30 to proline (A30P) and alanine 53 to threonine (A53T)), or increased synthesis of the normal a-synuclein. Additional genes for familial PD have been identified to date: mutations in parkin (PARK2), DJ-1 (PARK7), or PINK1 (PARK6) in familial forms of autosomal recessive parkinsonism and mutations in UCH-L1 (PARK5) and LRRK2/dardarin (PARK8) or genomic triplication of α-synuclein (PARK4) in familial forms of autosomal dominant parkinsonism.

Methods of analyzing for genetic aberrations have been described herein above.

Alzheimer's Disease:

Alzheimer's disease (AD) is a common chronic neurodegenerative disease and is the most common cause of dementia. The two hallmarks of the disease are senile plaques, which are mainly composed of extracellular deposits of amyloid β and neurofibrillary tangles, which consist of intracellular aggregates of aberrantly phosphorylated tau protein. The existence of the senile plaques is also associated with an inflammatory response which is considered to play a prominent and early role in AD.

In both types of AD, cell types such as astrocytes, neurons and microglia have been implicated in the pathogenesis of the disease.

In order to establish whether a mesenchymal stem cell population is suitable for autologous transplantation for the treatment of AD, astrocytes which have been differentiated from the patient may be contacted (e.g. co-cultured) with neuronal cells. The neurons may be primary neurons or cell lines of neurons. According to a particular embodiment, the neurons are differentiated from the mesenchymal stem cell population from which the astrocytes have been differentiated.

Preferably, the cytotoxic effect of the lineage specific cells on the neurons is analyzed.

According to a particular embodiment, microglia cells differentiated from CD34+ cells derived from the patient are co-cultured with the neurons either in the presence or absence of the mesenchymal cell derived astrocytic cells.

Methods of differentiating CD34+ cells are described herein above.

Methods of analyzing cytotoxicity are described herein above.

In addition to cell death, the neurons may be examined for susceptibility to oxidative stress, proteasome inhibition, mitochondria function and ER stress response following co-culture with the lineage specific cells differentiated from the patient MSC populations.

If the lineage specific cells derived from the patient are more cytotoxic to neurons than the identical lineage specific cells derived from a control subject, the MSC population from which they are derived may be deemed as unsuitable for autologous cell transplantation for the treatment of AD.

According to one embodiment, when the autologous MSC population is greater than 10% more cytotoxic than the control MSC population, the patient cell population is deemed unsuitable for autologous cell population for the treatment of AD.

According to one embodiment, when the autologous MSC population is greater than 20% more cytotoxic than the control MSC population, the patient cell population is deemed unsuitable for autologous cell population for the treatment of AD.

According to one embodiment, when the autologous MSC population is greater than 30% more cytotoxic than the control MSC population, the patient cell population is deemed unsuitable for autologous cell population for the treatment of AD.

According to one embodiment, when the autologous MSC population is greater than 40% more cytotoxic than the control MSC population, the patient cell population is deemed unsuitable for autologous cell population for the treatment of AD.

According to one embodiment, when the autologous MSC population is greater than 50% more cytotoxic than the control MSC population, the patient cell population is deemed unsuitable for autologous cell population for the treatment of AD.

The control MSC population is typically derived from a subject who does not have Alzheimer's disease, preferably a healthy subject. Age and sex matching of MSC populations is also contemplated.

As well as the co-culture assay described herein above, the present invention further contemplates analyzing for a genetic aberration of the MSCs originating from the subject. If the genetic aberration is detected, then the MSC population is not selected as a source for cell therapy. Thus, for example the present invention contemplates analyzing for the existence of a mutation in

The majority of Alzheimer's disease cases are sporadic, however there are rare cases of dominantly inherited familial forms of Alzheimer's disease that have mutations or a duplication of APP (encodes the amyloid-13 precursor protein), or mutations in the presenilin genes (which encode proteolytic enzymes that cleave APP into amyloid-13).

Methods of analyzing for genetic aberrations have been described herein above.

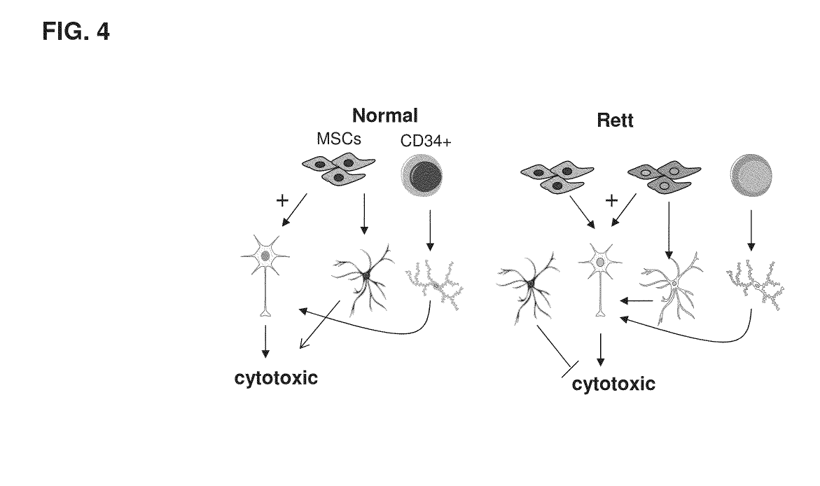

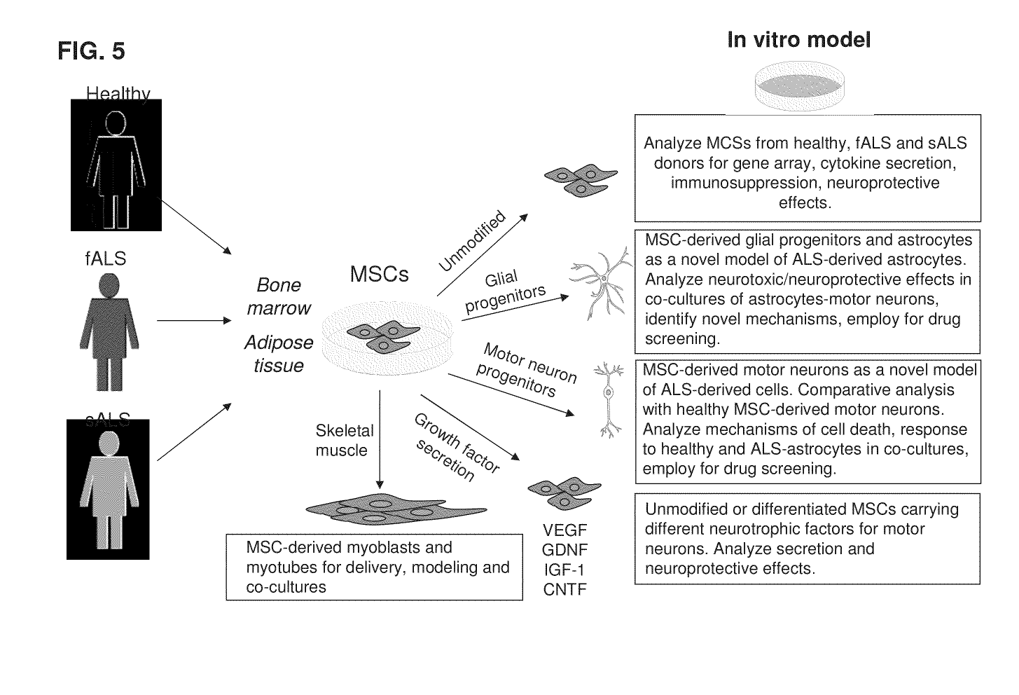

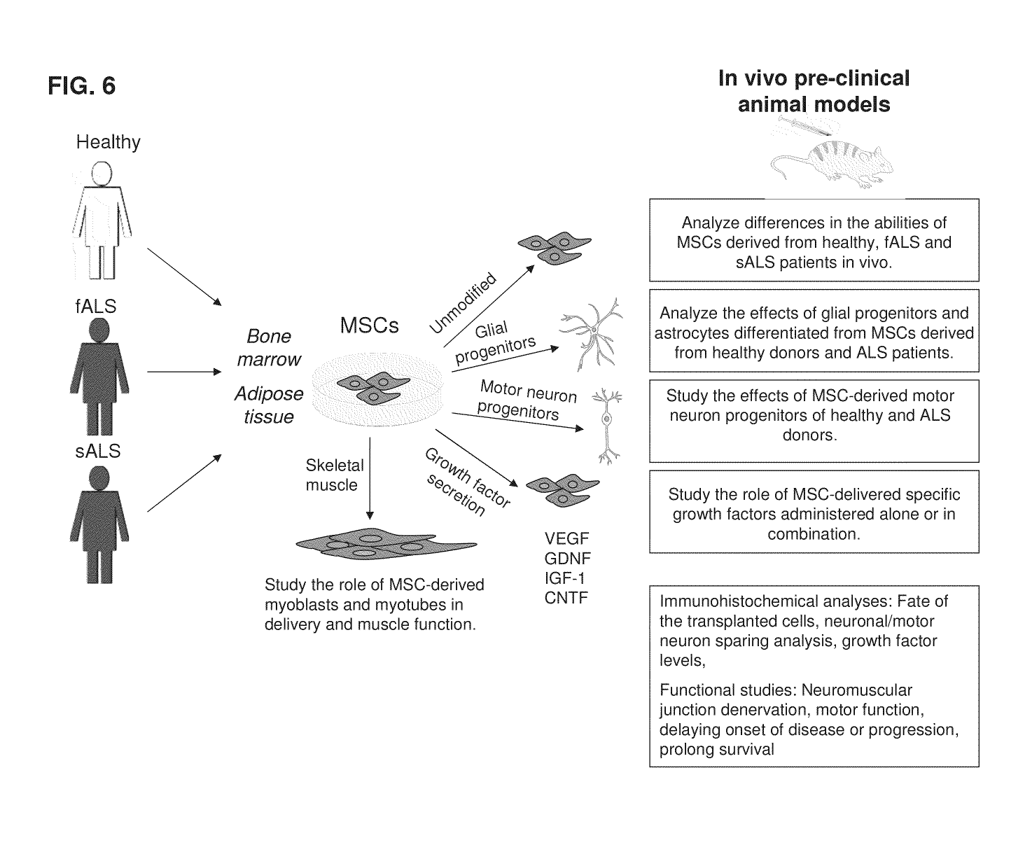

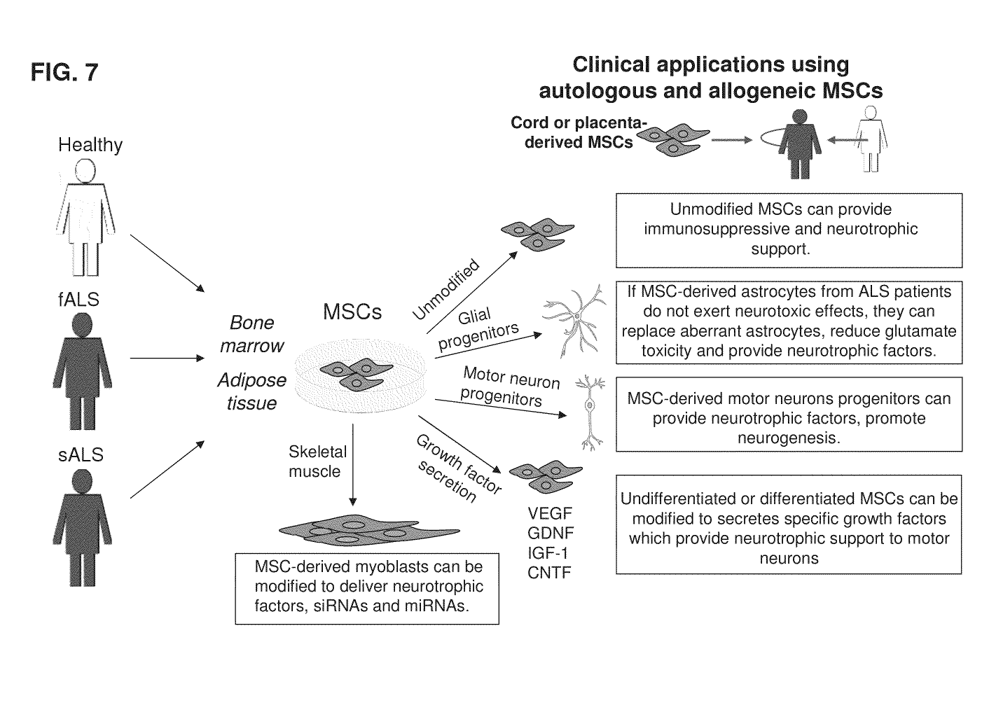

Rett Syndrome—See FIG. 4

Rett syndrome (RTT) is an X-chromosome linked autism spectrum disorders that is caused by loss of function of methyl cpG-binding protein 2 (MsCP2). Although earlier studies concluded that the disease is due to the loss of MeCP2 function in neurons, recent studies clearly demonstrated that the loss of MeCP2 in glial cells negatively affected neurons in a non-cell autonomous manner. Moreover, re-expression of MeCP2 in astrocytes in globally MeCP2-deficient mice significantly improved locomotion and anxiety levels, restored respiratory abnormalities to a normal pattern, and greatly prolonged lifespan compared to null mice. In addition, restoration of MeCP2 to the mutant astrocytes exerted a non-cell autonomous positive effect on mutant neurons in vivo, restoring normal dendritic morphology and increasing levels of the excitatory glutamate transporter. Therefore, it appears that glial cells, similar to neurons are integral components of the neuropathology of RTT, and support the idea of administration of glial cells with normal MeCP2 levels as a strategy for improving the associated symptoms of the disease.

In addition to astrocytes, microglia cells have been also implicated in the pathogenesis of the disease by secreting high levels of glutamate which is neuro-cytotoxic.

In order to establish whether a mesenchymal stem cell population is suitable for autologous transplantation for the treatment of Rett syndrome, astrocytes which have been differentiated from the patient may be contacted (e.g. co-cultured) with neurons. The neurons may be primary neurons or cell lines of neurons. According to a particular embodiment, the neurons are differentiated from the mesenchymal stem cell population from which the astrocytes have been differentiated.

Preferably, the cytotoxic effect of the lineage specific cells on the neurons is analyzed.

According to a particular embodiment, microglia cells differentiated from CD34+ cells derived from the patient are co-cultured with the neurons either in the presence or absence of the mesenchymal cell derived astrocytic cells.

Methods of differentiating CD34+ cells into a microglia phenotype are known in the art and are described herein above.

Methods of analyzing cytotoxicity have been described herein above.

In addition to cell death, the neurons may be examined for susceptibility to oxidative stress, proteasome inhibition, mitochondria function and ER stress response following co-culture with the lineage specific cells differentiated from the patient MSC populations.

If the lineage specific cells derived from the patient are more cytotoxic to neurons than the identical lineage specific cells derived from a control subject, the MSC population from which they are derived may be deemed as unsuitable for autologous cell transplantation for the treatment of Rett syndrome.

According to one embodiment, when the autologous MSC population is greater than 10% more cytotoxic than the control MSC population, the patient cell population is deemed unsuitable for autologous cell population for the treatment of Rett syndrome.

According to one embodiment, when the autologous MSC population is greater than 20% more cytotoxic than the control MSC population, the patient cell population is deemed unsuitable for autologous cell population for the treatment of Rett syndrome.

According to one embodiment, when the autologous MSC population is greater than 30% more cytotoxic than the control MSC population, the patient cell population is deemed unsuitable for autologous cell population for the treatment of Rett syndrome.

According to one embodiment, when the autologous MSC population is greater than 40% more cytotoxic than the control MSC population, the patient cell population is deemed unsuitable for autologous cell population for the treatment of Rett syndrome.

According to one embodiment, when the autologous MSC population is greater than 50% more cytotoxic than the control MSC population, the patient cell population is deemed unsuitable for autologous cell population for the treatment of Rett syndrome.

The control MSC population is typically derived from a subject who does not have Rett syndrome, preferably a healthy subject. Age and sex matching of MSC populations is also contemplated.

The present invention further contemplates differentiating genetically modified MSC populations in order to determine whether such cell populations are beneficial for autologous cell therapy. Thus for example, for ALS, the cells may be genetically modified to express IGF-1, VEGF, GDNF and/or SOD1 siRNA. For Parkinson's the cells may be genetically modified to express VEGF and GDNF. For Retts syndrome, the cells may be genetically modified to express IGF-1 and/or BDNF. Methods of genetically modifying MSCs are provided herein above.

It will be appreciated that as well as analyzing the effect of the lineage specific cell generated from the patients MSCs on another cell type, other parameters may also be analyzed in order to classify and/or qualify a MSC population.

Thus, according to another aspect of the present invention, there is provided a method of qualifying a population of mesenchymal stem cells (MSCs), the method comprising analyzing a function and/or morphology of a lineage-specific cell differentiated from the population of the MSCs, wherein an alteration in the function or morphology of the lineage-specific cell compared to a control lineage specific cell is indicative of a qualification of the MSCs.

Control lineage specific cells to which the differentiated cell types may be compared include but are not limited to astrocytes, motor neurons, oligodendrocytes and dopaminergic neurons. The control cells may be cultured cells or non-cultured, from a cell line or primary cells. The control cells may be differentiated ex vivo from MSC populations of healthy patients, as described herein above. Knowledge of functions and or morphologies of such control lineage specific cells may be gleaned from practical measurements or may be taken from the literature related to these cells.

According to one embodiment, the cells are qualified to see if they are suitable for autologous cell therapy of a brain disease in a subject.

The degree of alteration of the function or morphology is typically determined by the practitioner, preferably on a quantitative level, wherein a change of more than 10% in either direction, more than 20% in either direction, more than 30% in either direction, more than 40% in either direction, more than 50% in either direction, of the parameter being measured compared with the control lineage specific cell is indicative that the MSC cell population is not recommended for autologous cell transplant.

Cell morphology may be analyzed using microscopic techniques (e.g. scanning electro microscopy). Antibodies or dyes may be used to highlight distinguishing features in order to aid in the analysis.

Exemplary cell functions that may be analyzed include for example expression of a particular protein or set of proteins (including a relative amount of expression), expression of a particular miRNA or set of miRNAs, secretion of a particular factor (e.g. neurotrophic factor), an ability to respond to a particular factor (e.g. neurotrophic factor) and an ability to effect another cell-type (as described herein above).

According to a particular embodiment, the cell functions are analyzed on the RNA or protein level.

Methods of Detecting the Expression Level of RNA

The expression level of the RNA in the cells of some embodiments of the invention can be determined using methods known in the arts.

Northern Blot Analysis:

This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR Analysis:

This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

RNA In Situ Hybridization Stain:

In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the bound probe is detected using known methods. For example, if a radio-labeled probe is used, then the slide is subjected to a photographic emulsion which reveals signals generated using radio-labeled probes; if the probe was labeled with an enzyme then the enzyme-specific substrate is added for the formation of a colorimetric reaction; if the probe is labeled using a fluorescent label, then the bound probe is revealed using a fluorescent microscope; if the probe is labeled using a tag (e.g., digoxigenin, biotin, and the like) then the bound probe can be detected following interaction with a tag-specific antibody which can be detected using known methods.

In Situ RT-PCR Stain:

This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, Calif.).

DNA Microarrays/DNA Chips:

The expression of thousands of genes may be analyzed simultaneously using DNA microarrays, allowing analysis of the complete transcriptional program of an organism during specific developmental processes or physiological responses. DNA microarrays consist of thousands of individual gene sequences attached to closely packed areas on the surface of a support such as a glass microscope slide. Various methods have been developed for preparing DNA microarrays. In one method, an approximately 1 kilobase segment of the coding region of each gene for analysis is individually PCR amplified. A robotic apparatus is employed to apply each amplified DNA sample to closely spaced zones on the surface of a glass microscope slide, which is subsequently processed by thermal and chemical treatment to bind the DNA sequences to the surface of the support and denature them. Typically, such arrays are about 2×2 cm and contain about individual nucleic acids 6000 spots. In a variant of the technique, multiple DNA oligonucleotides, usually 20 nucleotides in length, are synthesized from an initial nucleotide that is covalently bound to the surface of a support, such that tens of thousands of identical oligonucleotides are synthesized in a small square zone on the surface of the support. Multiple oligonucleotide sequences from a single gene are synthesized in neighboring regions of the slide for analysis of expression of that gene. Hence, thousands of genes can be represented on one glass slide. Such arrays of synthetic oligonucleotides may be referred to in the art as “DNA chips”, as opposed to “DNA microarrays”, as described above [Lodish et al. (eds.). Chapter 7.8: DNA Microarrays: Analyzing Genome-Wide Expression. In: Molecular Cell Biology, 4th ed., W.H. Freeman, New York. (2000)].

Oligonucleotide Microarray—

In this method oligonucleotide probes capable of specifically hybridizing with the polynucleotides of some embodiments of the invention are attached to a solid surface (e.g., a glass wafer). Each oligonucleotide probe is of to approximately 20-25 nucleic acids in length. To detect the expression pattern of the polynucleotides of some embodiments of the invention in a specific cell sample (e.g., blood cells), RNA is extracted from the cell sample using methods known in the art (using e.g., a TRIZOL solution, Gibco BRL, USA). Hybridization can take place using either labeled oligonucleotide probes (e.g., 5′-biotinylated probes) or labeled fragments of complementary DNA (cDNA) or RNA (cRNA). Briefly, double stranded cDNA is prepared from the RNA using reverse transcriptase (RT) (e.g., Superscript II RT), DNA ligase and DNA polymerase I, all according to manufacturer's instructions (Invitrogen Life Technologies, Frederick, Md., USA). To prepare labeled cRNA, the double stranded cDNA is subjected to an in vitro transcription reaction in the presence of biotinylated nucleotides using e.g., the BioArray High Yield RNA Transcript Labeling Kit (Enzo, Diagnostics, Affymetix Santa Clara Calif.). For efficient hybridization the labeled cRNA can be fragmented by incubating the RNA in 40 mM Tris Acetate (pH 8.1), 100 mM potassium acetate and 30 mM magnesium acetate for 35 minutes at 94° C. Following hybridization, the microarray is washed and the hybridization signal is scanned using a confocal laser fluorescence scanner which measures fluorescence intensity emitted by the labeled cRNA bound to the probe arrays.

For example, in the Affymetrix microarray (Affymetrix®, Santa Clara, Calif.) each gene on the array is represented by a series of different oligonucleotide probes, of which, each probe pair consists of a perfect match oligonucleotide and a mismatch oligonucleotide. While the perfect match probe has a sequence exactly complimentary to the particular gene, thus enabling the measurement of the level of expression of the particular gene, the mismatch probe differs from the perfect match probe by a single base substitution at the center base position. The hybridization signal is scanned using the Agilent scanner, and the Microarray Suite software subtracts the non-specific signal resulting from the mismatch probe from the signal resulting from the perfect match probe.

Methods of Detecting Expression and/or Activity of Proteins

Expression and/or activity level of proteins expressed in the cells of the cultures of some embodiments of the invention can be determined using methods known in the arts.

Enzyme Linked Immunosorbent Assay (ELISA):

This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western Blot:

This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-Immunoassay (RIA):

In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence Activated Cell Sorting (FACS):

This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical Analysis:

This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In Situ Activity Assay:

According to this method, a chromogenic substrate is applied on the cells containing an active enzyme and the enzyme catalyzes a reaction in which the substrate is decomposed to produce a chromogenic product visible by a light or a fluorescent microscope.

In Vitro Activity Assays:

In these methods the activity of a particular enzyme is measured in a protein mixture extracted from the cells. The activity can be measured in a spectrophotometer well using colorimetric methods or can be measured in a non-denaturing acrylamide gel (i.e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

Following differentiation to an astrocytic cell, the following exemplary parameters may be analyzed:

1. expression and/or secretion of a neurotrophic factor.

As used herein, the phrase “neurotrophic factor” refers to a cell factor that acts on the cerebral nervous system comprising growth, differentiation, functional maintenance and/or survival effects on neurons. Examples of neurotrophic factors include, but are not limited to, glial derived neurotrophic factor (GDNF), GenBank accession nos. L19063, L15306; nerve growth factor (NGF), GenBank accession no. CAA37703; neurotrophin-3 (NT-3), GenBank Accession No. M37763; neurotrophin-4/5; Neurturin (NTN), GenBank Accession No. NP_{—}004549; Neurotrophin-4, GenBank Accession No. M86528; Persephin, GenBank accession no. AAC39640; brain derived neurotrophic factor, (BDNF), GenBank accession no. CAA42761; artemin (ART), GenBank accession no. AAD13110; ciliary neurotrophic factor (CNTF), GenBank accession no. NP_{—}000605; insulin growth factor-I (IGF-1), GenBank accession no. NP_{—}000609; and Neublastin GenBank accession no. AAD21075.

2. enhancement of expression and/or secretion of a neurotrophic factor following addition of IL-1beta and cabergoline.

3. activity of glutamate transporters. The activity of such glutamate transporters may be analyzed by measuring labeled aspartate (e.g. [³H]-d-aspartate uptake from the culture medium of the cells.

4. an astrocytic structural phenotype including the presence of a round nucleus, a “star shaped” body and many long processes that end as vascular foot plates on the small blood vessels of the CNS. Further examples of structural astrocytic phenotypes may be found in the following materials: Reynolds and Weiss, Science (1992) 255:1707-1710; Reynolds, Tetzlaff, and Weiss, J. Neurosci (1992) 12:4565-4574; and Kandel, et al., Principles of Neuroscience, third ed. (1991), Appleton & Lange, Norwalk, Conn.

5. expression of an astrocyte marker.

As used herein the phrase “astrocyte marker” refers to a polypeptide which is either selectively or non-selectively expressed in an astrocyte. The astrocyte marker may be expressed on the cell surface or internally. Examples of astrocyte markers include 5100 beta, glial fibrillary acidic protein (GFAP), glutamine sythetase, GLAST and GLT1.

Following differentiation to a neuronal cell, the following parameters may be analyzed:

1. release of a neurotransmitter;

A neurotransmitter according to the teaching of the present invention can be any substance which is released on excitation from the axon terminal of a presynaptic neuron of the central or peripheral nervous system and travel across the synaptic cleft to either excite or inhibit the target cell. The neurotransmitter can be, for example, dopamine, norepinephrine, epinephrine, gamma aminobutyric acid, serotonin, acetylcholine, glycine, histamine, vasopressin, oxytocin, a tachykinin, cholecytokinin (CCK), neuropeptide Y (NPY), neurotensin, somatostatin, an opioid peptide, a purine or glutamic acid.

2. expression of a neuronal marker

neuronal marker such as, for example, a neural protein such as Glypican-4 (GPC4), Necdin, Nestin, Neurite growth-promoting factor 2 (NEGF-2), Neurofilament-heavy, Neurofilament-light, Neurofilament-medium, Neuron specific enolase (NSE), Neurotrophic tyrosine kinase receptor type 2 (TRK-2), Neuronal Nuclei (NeuN), RET tyrosine kinase or Retinoic acid receptor type .alpha. (RARA), and oligo-dendrocytes protein such as 2′,3′-Cyclic nucleotide 3′-phosphodiesterase (CNPase). Alternatively, the neuronal marker may be a neuronally active transcription factor such as, for example Aryl hydrocarbon receptor/Aryl hydrocarbon receptor nuclear translocator binding element (AhR/Amt), Ecotropic viral integration site 1 (EVI-1), Forkhead box O1A human (FKHRhu), Glycosaminoglycan (GAG), Hepatocyte nuclear factor 3.beta. (HNF-3.beta.), Myelin gene expression factor 2 MEF2(2), Nuclear Y box factor (NF-Y), Neural zinc finger 3 (NZF-3), Paired box gene 3 (Pax-3), Paired box gene 6 (Pax-6) or Xenobiotic response element (XRE). Preferably if the cells are required to treat Parkinson's disease, then they should also express a dopaminergic marker such as a dopaminergic transcription factor such as Aldehyde dehydrogenase 1 (Aldh1), Engrailed 1(En-1), Nun-1 or Paired-like homeodomain transcription factor 3 (PITX-3) or a dopaminergic protein such as Aromatic L-amino acid decarboxylase (AADC), Catechol-o-methyltransferase (COMT), Dopamine transporter (DAT), Dopamine receptor D2 (DRD2), GTP cyclohydrolase-1 (GCH), Monoamine oxidase B (MAO-B), Tryptophan hydroxylase (TPH), Vesicular monoamine transporter 2 (VMAT 2), Patched homolog (PTCH), Smoothened (SMO) or Tyrosine hydroxylase (TH).

3. cell morphology (e.g. possession of dendrites).

Following differentiation to an oligodendrocyte cell, the following parameters may be analyzed:

1. Expression of oligodendrocyte markers:

GaABC2/ABCA2: a specific marker for oligodendrocytes but not for myelinsheaths. as a lysosome-associated membrane protein that is being localized specifically in oligodendrocytes. detected only in the cell bodies of oligodendrocytes. detected mostly around lysosome and partly in Golgi apparatus by electron microscopy.

Amphoterin (P30, HMG-1) and RIP: early markers of oligodendrocytes in the developing rat spinal cord.

Carbonic Anhydrase II (CA II): a marker for oligodendrocytes, localized in oligodendrocytes.

CNPase: a marker for oligodendrocytes.

Galactocerebroside: a marker for oligodendrocytes; a specific cell-surface antigenic marker for oligodendrocytes in culture.

Microtubule-Associated Protein 4 (MAP-4): a marker for astroglia and oligodendroglia.

Myelin-Oligodendrocyte Glycoprotein (MOG): a surface marker of oligodendrocyte maturation.

NB3C4: a novel monoclonal antibody specific for oligodendrocytes.

Nestin: a marker of oligodendrocyte lineage cells.

Olig2: The oligodendroglial lineage marker, is expressed in neural progenitors and oligodendroglia and are essential for oligodendrocyte development. PMID: 15198128,

POP66: a paraneoplastic encephalomyelitis-related antigen, is a marker of adult oligodendrocytes.

Sulph I: identify oligodendrocyte progenitor cells.

Transferrin Binding Protein (TfBP): a marker for avian oligodendrocytes.

Transferrin: an oligodendrocyte-specific marker which is expressed earlier than galactocerebroside.

2. Ability to produce myelin; and

3. cell morphology (a branched and ramified phenotype and formation of myelin membranes).

It will be appreciated that the patient-derived differentiated and non-differentiated cell populations described herein may be used to screen for agents which may be used for the treatment of the brain disorders.

Thus, according to yet another aspect of the present invention there is provided a method of selecting an agent for the treatment of a brain disease, the method comprising analyzing an ability of a population of MSCs isolated from a patient with a brain disorder to differentiate towards a lineage-specific cell associated with the brain disorder in a presence and absence of the agent, wherein an improvement of a function or morphology of the lineage specific cell as compared to an identical lineage specific cell derived from a control population of MSCs in the presence of the agent is indicative that the agent may be used for the treatment of the brain disease.

Agents may further be screened following differentiation of the patient derived MSCs to a lineage specific cell. The effect of the lineage specific cell on a second lineage specific cell may be analyzed in the presence and absence of the agent. For example, ALS patient derived MSCs may be differentiated into astrocyte cells. The astrocyte cells may be co-cultured with neuronal cells (e.g. motor neurons) in the presence and absence of the putative agent. An agent which decreases the cytotoxic effect of the astrocyte cells on the neuronal cells may be advantageous for the treatment of ALS.

Exemplary agents that may be screened include nucleic acids, e.g., polynucleotides, ribozymes, siRNA and antisense molecules (including without limitation RNA, DNA, RNA/DNA hybrids, peptide nucleic acids, and polynucleotide analogs having altered backbone and/or bass structures or other chemical modifications); proteins, polypeptides (e.g. peptides), carbohydrates, lipids and “small molecule” drug candidates. “Small molecules” can be, for example, naturally occurring compounds (e.g., compounds derived from plant extracts, microbial broths, and the like) or synthetic organic or organometallic compounds having molecular weights of less than about 10,000 daltons, preferably less than about 5,000 daltons, and most preferably less than about 1,500 daltons.

The agent may be added to the culture medium during differentiation of the patient MSCs. It will be appreciated that when the agent is a polypeptide or polynucleotide (e.g. miRNA or polynucleotide agent which hybridizes with and down-regulates the function of a miRNA), the patient MSCs may be genetically modified to express the agent. Methods of genetically modifying cells are described herein above.

Exemplary morphologies that may be analyzed for particular cell types and methods of performing the analyses are described herein above.

Exemplary cell functions that may be analyzed include for example expression of a particular protein or set of proteins (including a relative amount of expression), expression of a particular miRNA or set of miRNAs, secretion of a particular factor (e.g. neurotrophic factor), an ability to respond to a particular factor (e.g. neurotrophic factor) and an ability to effect another cell-type (as described herein above).

The present inventors have shown that MSC populations derived from a patient may comprise a mutation that is associated with a particular disease—see Example 1, herein below. The present invention contemplates introducing mutations associated with these diseases into normal MSC populations (or performing gene silencing) and differentiating these mutated populations into lineage specific cells. Such lineage specific cells can be used instead of or in parallel with the lineage specific cells derived from patient MSCs as disease models or to screen for new drugs.

The patient mesenchymal stem cell populations described herein may be stored individually or may be comprised in a bank, each population being categorized according to a particular parameter.

Thus, according to still another aspect of the present invention there is provided a method of producing a mesenchymal stem cell (MSC) bank comprising: harvesting undifferentiated mesenchymal stem cells from a plurality of subjects which have a brain disorder to obtain a plurality of separate cell populations; differentiating the mesenchymal stem cells towards a lineage specific cell; storing the undifferentiated mesenchymal stem cells and the lineage specific cells thereby producing a mesenchymal stem cell bank.

The mesenchymal stem cell bank of this aspect of the present invention is a physical collection of one or more MSC population (both differentiated and undifferentiated) derived from patients with a brain disorder. Such banks preferably contain more than one sample (i.e., aliquot) of each MSC population. Harvesting undifferentiated mesenchymal stem cells is described herein above. The MSC populations may be derived from various sources including bone marrow, adipose tissue, cord blood and placenta. The differentiated MSC populations may be generated according to the methods described herein. The bank may also contain one or more samples of the human feeder cells and/or human serum used to expand and/or differentiate the MSC populations.

The MSC populations are stored under appropriate conditions (typically by freezing) to keep the stem cells alive and functioning. According to one embodiment, the MSC populations are stored as cryopreserved populations. Other preservation methods are described in U.S. Pat. Nos. 5,656,498, 5,004,681, 5,192,553, 5,955,257, and 6,461,645. Methods for banking stem cells are described, for example, in U.S. Patent Application Publication No. 2003/0215942.

According to one embodiment, the undifferentiated cell populations stored in the bank are characterized according to at least one predetermined characteristic.

Additionally, the differentiated cell population stored in the bank may also be characterized according to at least one predetermined characteristic.

Predetermined characteristics include, but are not limited to morphological characteristics, differentiation profile, blood type, major histocompatibility complex, disease state of donor, or genotypic information (e.g. single nucleated polymorphisms, ‘SNPs’ of a specific nucleic acid sequence associated with a gene, or genomic or mitochondrial DNA) associated or not associated with the disease.

According to a specific embodiment, the predetermined characteristic of the differentiated cells comprises an effect on a second population of lineage specific cells (as described herein above).

Cataloguing may constitute creating a centralized record of the characteristics obtained for each cell population, such as, but not limited to, an assembled written record or a computer database with information inputted therein. The stem cell bank facilitates the selection from a plurality of samples of a specific mesenchymal stem cell sample suitable for a researcher's or clinician's needs.

According to one embodiment, the mesenchymal stem cell bank described herein is maintained by a stem cell database computer unit. Each computer unit comprises at least one processing module, respectively, for processing information. The computer unit may be communicatingly connected to a display. Information directed to mesenchymal stem cell populations may be stored on a database computer which is conveyed to users via a network connection. Such a system provides the customer the ability to evaluate the mesenchymal stem cell populations to determine which are suitable for their ongoing research and use and may also serve to facilitate the transaction of purchasing stem cells and proper shipment.

As will be appreciated by one of skill in the art, embodiments of the present invention may be embodied as a device or system comprising a processing module, and/or computer program product comprising at least one program code module. Accordingly, the present invention may take the form of an entirely hardware embodiment or an embodiment combining software and hardware aspects. Furthermore, the present invention may include a computer program product on a computer-usable storage medium having computer-usable program code means embodied in the medium. Any suitable computer readable medium may be utilized including hard disks, CD-ROMs, DVDs, optical storage devices, or magnetic storage devices.

The term “processing module” may include a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on operational instructions. The processing module may have operationally coupled thereto, or integrated therewith, a memory device. The memory device may be a single memory device or a plurality of memory devices. Such a memory device may be a read-only memory, random access memory, volatile memory, non-volatile memory, static memory, dynamic memory, flash memory, and/or any device that stores digital information. A computer, as used herein, is a device that comprises at least one processing module, and optionally at least one memory device.

The computer-usable or computer-readable medium may be or include, for example, but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM), a CD ROM, a DVD (digital video disk), or other electronic storage medium. Note that the computer-usable or computer-readable medium could even be paper or another suitable medium upon which the program is printed, as the program can be electronically captured, via, for instance, optical scanning of the paper or other medium, then compiled, interpreted or otherwise processed in a suitable manner if necessary, and then stored in a computer memory.

Computer program code for carrying out operations of certain embodiments of the present invention may be written in an object oriented and/or conventional procedural programming languages including, but not limited to, Java, Smalltalk, Pert, Python, Ruby, Lisp, PHP, “C”, FORTRAN, or C++. The program code may execute entirely on the user's computer, partly on the user's computer, as a stand-alone software package, partly on the user's computer and partly on a remote computer or entirely on the remote computer. In the latter scenario, the remote computer may be connected to the user's computer through a local area network (LAN) or a wide area network (WAN), or the connection may be made to an external computer (for example, through the Internet using an Internet Service Provider).

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

It is noted that for each miR described herein the corresponding sequence (mature and pre) is provided in the sequence listing which should be regarded as part of the specification.

Various embodiments and aspects of the present invention as delineated hereinabove and as claimed in the claims section below find experimental support in the following examples.

### EXAMPLES

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### Example 1

### Amyotrophic Lateral Sclerosis

Amyotrophic lateral sclerosis (ALS) is a fatal neurodegenerative disease of unknown cause affecting the upper and lower motor neurons of patients. Approximately 5,600 people in the U.S. are diagnosed with ALS each year. The incidence of ALS is two per 100,000 people, and it is estimated that as many as 30,000 Americans may have the disease at any given time. 80% of patients die within five years following diagnosis, where normally death is due to respiratory failure. To this day, there is no effective treatment for ALS; the only drug currently approved by the FDA is a NMDA receptor antagonist (riluzole) that increases the rate of survival by 6 months. It is imperative to find new drugs for this disease and try to better understand the causes and mechanism of action, which are still largely unknown.

The three cell types that are involved in the pathogenesis of ALS are motor neurons, skeletal muscle cells and astrocytes. These cells are involved in pathogenesis of both familial (fALS) and sporadic ALS (sALS). However, although it is known that mutations in SOD1 gene may play a role at least in some of the fALS patients, it is currently not known what are the molecular mechanisms that play a role in sALS and in the rest of the fALS patients. Although SOD-mice represent an acceptable experimental model for studying novel approaches for the treatment of ALS, the relevance of SOD-mice to the fALS remains questionable. Moreover, no experimental animal models exist for studying sALS which represent the large majority of the cases of ALS. The optimal approach would be to study the mechanisms of motor neuron degeneration using cells derived from fALS and sALS patients; however it is practically impossible to isolate all the relevant cell types from these patients. A potential solution is to generate iPS and differentiate them to the relevant cell types. Although it has been possible to successfully generate and differentiate ALS-derived iPS to motor neurons, limitations for these technique are the expression of embryonic genes that may introduce erroneous genotypic and phenotypic characteristics, and the inability to directly apply the results of these studies to the clinic since differentiated iPS can not be yet used clinically.

The present inventors have recently developed a novel approach to differentiate bone marrow and adipose tissue-derived mesenchymal stromal stem cells (MSCs) into astrocytes, skeletal muscle cells and motor neuron progenitors and propose to employ the ALS-derived MSCs in the following settings:

1. Whole Genome and miRNA Analysis.

Perform whole genome and miRNA analysis using microarrays of both undifferentiated MSCs and MSCs differentiated to motor neurons, astrocytes and skeletal muscle cells to identify differences in these cells as compared to similar cells derived from normal individuals. These results can be then used for the identification of novel mechanisms that may play a role in the pathogenesis of the disease as well as novel therapeutic targets. Moreover, since replacement therapy by differentiated MSCs may represent the most rational therapeutic approach for patients with ALS, these results may have clinical implications in deciding which cell type should be used clinically for treatment of patients with ALS. Indeed, our preliminary studies using miRNA microarray analysis have identified some changes in the expression of specific miRNAs in BM-MSCs derived from ALS and normal individuals.

2. Differentiation of MSCs to Astrocytes, Skeletal Muscle Cells and Motor Neurons.

The novel approach to differentiate both bone marrow and adipose-derived MSCs to all the cells relevant to ALS from normal and ALS patients provide the present inventors with a unique ability to generate an in vitro model of this disease. This model of both fALS and sALS may be used to delineate molecular mechanisms (gene and miRNA arrays), phenotypic changes and different functional assays. It can also allow the inventors to delineate the role of each specific cell type, alone and in combination, in the pathogenesis of the disease. In addition, this model can be used to develop high-throughput cell-based assays to screen for small molecules that may promote survival of motor neurons (antagonize the effect of mSOD1, or provide neurotrophic support), prevent skeletal muscle atrophy or alter the function of impaired astrocytes in ALS patients.

In addition, ALS-derived MSCs can be modified by introducing specific SOD or other relevant mutants or silence mutated SOD or other relevant genes and analyze the functions of the different differentiated cells.

As such, the ability to differentiate MSCs to all relevant cell types that may play a role in the pathogenesis of ALS, can provide a practical approach for the specific identification of the relevant molecular and cellular abnormalities of all patients with ALS or on a fully personalized basis and provide a clue for an optimal therapeutic approach, using either patient's own MSCs, healthy family member's MSCs or MSCs derived from umbilical cord or placenta for treatment.

3. Clinical Applications of MSCs in ALS Patients.

Some studies demonstrated the safety of using patient-derived MSCs in neurodegenerative diseases such as multiple sclerosis and ALS. However, the therapeutic efficiency of these cells is limited. Therefore the use of differentiated MSCs which can replace specific cell populations or provide specific neurotrophic factors that may improve the abnormal function of patient's cells seems a more efficient approach. The studies of the in vitro model can provide the clinician with important information regarding the pathogenesis of the disease and the therapeutic targets that may be accomplished by autologous or allogeneic differentiated cells that may be used for efficient treatment of patients in need. Indeed, a most important point that should be considered once the pathogenesis of ALS will be better understood, is whether or not patient-derived MSCs should be used therapeutically or whether optimal treatment of ALS may depend on the use of MSCs obtained from a normal donor, possibly even from a universal pool of MSCs derived from unrelated placenta or cord that can be used off-the-shelf as ready-made therapeutic cells, as soon as indicated, avoiding loss of time that may be required for differentiation of patient's own or family member's MSCs. Recent studies demonstrated that astrocytes derived from both fALS and sALS may exert neurotoxic effects on motor neurons, suggesting that cells derived from the patient, including differentiated MSCs, may exhibit a phenotype that is likely to impair their therapeutic benefits. Therefore, analyzing the function of unmodified and differentiated MSCs in the context of the cells relevant to the disease as compared to similar cells obtained from normal individuals may provide important information that will have direct clinical implications as to the optimal cellular source for treatment of the patients.

In case differentiated autologous MSCs may not be suitable for treatment of patients with ALS, fully MHC-matched or haploidentical MSCs obtained from a normal family member may represent a better alternative. In fact, as indicated above, considering the low expression of MHC, particularly MHC class II on the cell surface of MSCs and considering their immunosuppressive properties that can prevent their rejection, the use of differentiated MSCs prepared from placenta and cord's Wharton's jelly may provide an ideal “off-the-shelf” treatment for patients with ALS as well as other neurodegenerative disorders. In this regards it should be mentioned that placenta and cord derived MSCs can be equally well differentiated into all relevant cell types

The potential use of unrelated allogeneic MSCs can be examined in SOD-mice that currently represent an animal model of fALS using unmodified and differentiated MSCs derived from syngeneic, H-2 identical, haploidentical or fully MHC mismatched donors, as well as by comparing the therapeutic benefit of treating mice with MSCs derived from ASL patients in comparison with normal MSCs derived from placenta or cord.

4. MSCs as Delivery Tools in ALS.

MSCs have been shown to deliver various factors to neighboring cells such as growth factors, viruses, siRNAs and miRNAS. An important concept is that the innervation of a tissue is dependent on signaling inputs, such as neurotrophic factors, secreted by the innervated tissue. Thus, our hypothesis is that MSC-derived skeletal muscle cells may be the optimal cells to deliver trophic factors and relevant siRNA or miRNA to the degenerated motor neurons, especially in early stages of ALS, when motor neurons are still functional In addition to skeletal muscle, astrocytes are another cell type that play an important role in providing neurotrophic support and removing neurotoxic factors or excess excitatory neurotransmitters such as glutamate from the neuronal microenvironment. The present inventors therefore propose to use our ALS-in vitro model for analyzing the ability of MSC-derived skeletal muscle cells or astrocytes to deliver specific neurotrophic factors that are important for the function and survival of motor neurons such as GDNF, VEGF and IGF-1 and remove neurotoxic factors.

In addition, recent studies suggest that silencing of SOD1 may provide therapeutic benefit in ALS patients; however limitation to this type of therapy is the mode of delivery. It has recently demonstrated that MSCs can efficiently deliver both siRNAs and synthetic miRNA mimics to neighboring cells. Thus the use of the in vitro model for screening both the therapeutic potential of SOD1 or other relevant siRNAs as well as relevant miRNA that have been implicated in the pathogenesis of ALS (such as miR-206 and miR-1 that are also important in myogenesis) is suggested. Combination of specific neurotrophic factors and siRNAs/miRNAs may also be tested as well.

5. Development of Similar In Vitro Models to Additional Neurodegenerative Disorders.

Similar models as proposed for ALS can be developed for studying the pathogenesis and desirable therapeutic approach for additional neurodegenerative disorders. For example, we were able to generate dopaminergic neurons from MSCs. These neurons may be employed to identify novel molecular mechanisms that contribute to the pathogenesis of Parkinson's disease and to provide a high throughput system for drug screening or for designing optimal cellular-based treatment of Parkinson's disease.

Similar in vitro models can be developed for studying the pathogenesis of autism spectrum disorder (ASD) which probably represent a family of disorders each caused by a different unknown pathogenesis, Rett syndrome, Alzheimer's disease, Huntington's disease and additional neurodegenerative disorders of unknown cause and with no effective treatment available, focusing on disease-specific abnormalities or patient-specific defects. Taken together, the feasibility to generate cells resembling the damaged or the abnormal tissue of a given disease may provide a new disease-specific or even personalized approach for better diagnosis and optimal treatment of many diseases currently considered incurable, based on relevant disease specific models towards new drug design or paving the road towards optimal cellular treatment.

MSCs Derived from ALS Patients Express Different Gene and miRNAs then MSCs-Derived from Healthy Controls:

In order to examine whether MSCs derived from ALS patients are different from MSCs that are derived from healthy controls, the present inventors compared 7 MSC cultures from ALS patients and 7 MSC cultures from healthy controls.

Firstly, DNA samples from the MSCs were examined for mutations in SOD1 and in an additional gene, TARDBP, a protein that has been reported to be mutated and to play a role in the pathogenesis of some sporadic ALS patients.

None of the 7 MSCs that were examined carried a SOD1 mutation. In contrast, one of the ALS patients carried a mutation in the TARDBP gene.

Next, gene and miRNA arrays were performed on the MSCs from the ALS and normal controls in order to identify differences in their gene and miRNA expression. Using gene array (Agilent Array 4×44K), 164 genes were identified that were differentially expressed in the ALS and control-derived MSCs.

A partial list is presented below.

Downregulated Genes
1. BMP6—plays a role in the survival of motor neurons (−2.8).
2. Melanocortin 2—exerts neurotrophic effect in the spinal cord (−3.5).
3. MARKSL1—determines actin stability and neuronal migration (−3.8).
4. Fibrillin 2—plays a role in TGF-beta signaling and in muscle function (−4.3).
5. COL5A3—decreased in muscle atrophy (−4.4)

6. DAP12—requires for removal of dead neurons without inflammation, important for myelination and synaptic activity (−5.2).

Upregulated Genes

1. Cytoplasmic alpha actin (+2.9)

2. Human aldo-keto reductase (+6.8)

3. DYNLT1 dynein, light chain, Tctex-type 1 (+8.9)

Using miRNA analysis, 14 miRNAs were identified that were differentially expressed in the two groups of MSCs, some of which are brain or muscle specific or associated with generation of ROS or dysfunction of the BNDA receptor.

miR-17-5p

miR-372

miR-373

miR-146b

miR-181a

miR-181d

miR-124a-brain specific, induces neuronal differentiation

miR-128a-regulates ROS generation

miR-128b-brain specific

miR-135b

miR-183

miR-219-Associated with NMDA receptor dysfunction

miR-133a-muscle specific

miR-133b

Collectively, these results further demonstrate that MSCs derived from ALS patients differ in the expression of their genes and miRNAs and that some of these differences may have relevance to the pathogenesis of ALS.

Conditioned Medium of ALS-Derived MSCs Differentiated to Astrocytes Induces Cell Death of Motor Neurons.

Recent studies implicated astrocytes in the pathogenesis of ALS. These studies demonstrated that co-cultures of astrocytes derived from ALS patients with motor neurons derived from healthy individuals induced cell death of these cells. Similar results were obtained with conditioned media derived from the ALS-derived MSCs that were differentiated into astrocytes (Diaz-Amarillo P., Proc Natl Acad Sci USA. 2011 Nov. 1; 108(44):18126-31; and Haidet-Phillips A M., Nat Biotechnol. 2011 Aug. 10; 29(9):824-8).

To examine the ability of differentiated MSC-derived neural cells to serve as an in vitro model for ALS, five MSC populations derived from ALS patients were differentiated into astrocytes and five MSC populations derived from healthy subjects were differentiated into astrocytes. The effects of their conditioned medium on the cell death of cultured motor neurons were examined. Motor neurons were generated from neural stem cells and were characterized by the expression of specific markers such as HB9, islet1 and ChAT.

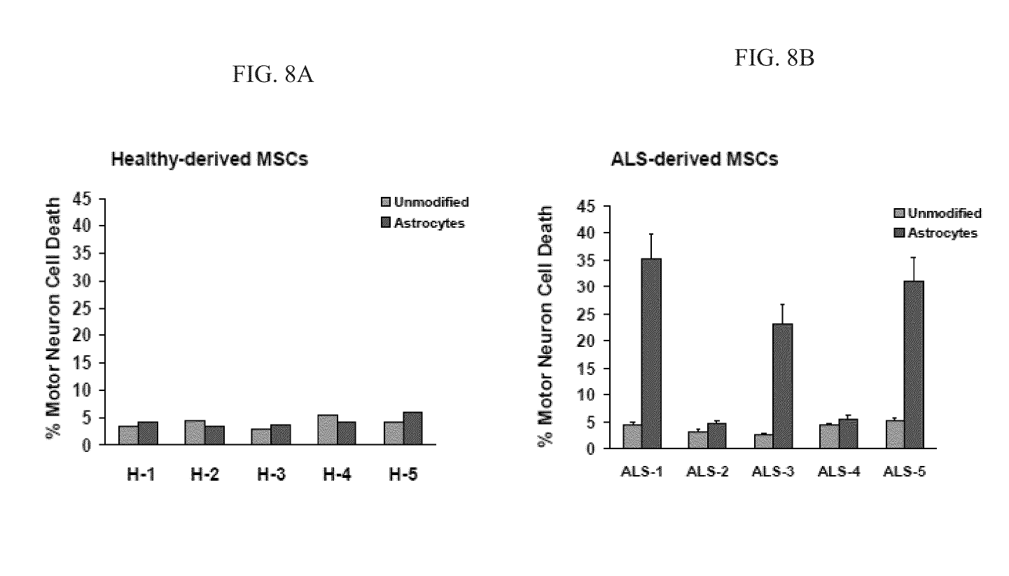

As illustrated in FIGS. 8A-B, conditioned media of unmodified MSCs derived from healthy control or ALS patients did not exert cell death of the motor neuron cultures. In contrast, when differentiated into astrocytes, three out of the five ALS-derived MSCs induced a significant cell death of the motor neurons, whereas, none of the control MSCs induced a significant of cell death. These results indicate that similar to the neurotoxic effect of ALS-derived astrocytes, astrocyte-differentiated MSCs can induce motor neuron cell death and therefore can be employed as an efficient in vitro system for studying disease mechanisms and for high throughput drug screening in this disorder.

These results have also important and immediate implications for the therapeutic use of MSCs in this disease and suggest that allergenic MSCs may be a better choice for the treatment of these patients.

To further examine the feasibility of using MSC-derived astrocytes as an in vitro model in ALS, bone marrow MSCs having either a wt SOD1 or a mutant SOD1 (SOD1G93A) were differentiated into astrocyte like cells and their effect examined on the cytotoxic effects of unmodified MSCs and MSC differentiated into motor neurons.

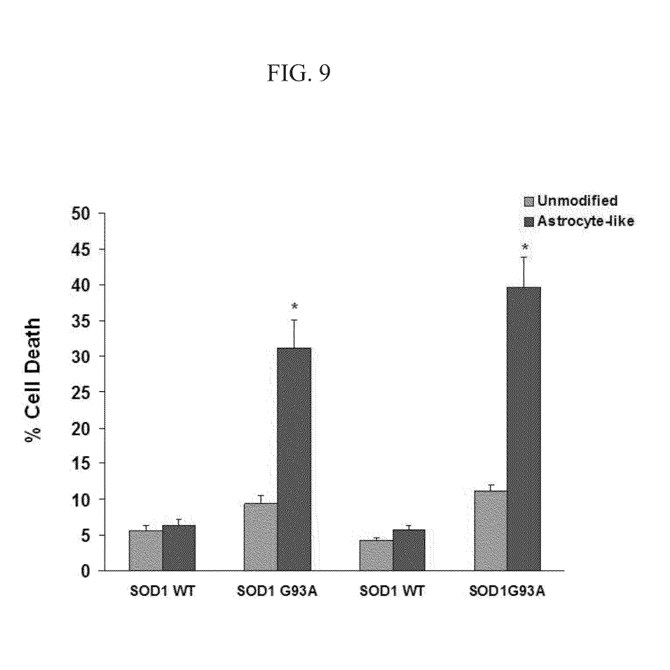

As presented in FIG. 9, MSC-derived astrocyte like cells expressing a SOD1 G93A mutant induced cell death of motor neurons, whereas unmodified MSCs did not have a significant effect. These results indicate that astrocyte-like cells derived from MSCs carrying a SOD1 G93A mutant exhibit similar characteristics to ALS-derived astrocytes and further demonstrate the feasibility of using these cells as an in vitro model for this disease.

### Example 2

### Parkinson's Disease

Parkinson's disease (PD) is a progressive neurodegenerative disease that is characterized by rigidity, bradykinesia and resting tremor. The pathological hallmarks of this disease are the selective loss of dopaminergic neurons in the substantia nigra pars compacta (SNc) in the midbrain. Most of the cases of PD occur sporadically with yet unknown causes and pathogenesis. Potential mechanisms include mitochondrial dysfunction, oxidative stress, endoplasmic reticulum stress, failure of the ubiquitin-proteasome system, some environmental factors, and genetic predisposition.

In addition to the sporadic form of PD, there is also a familial form which is observed in about 5% of all PD patients. Although most of the PD cases are sporadic, multiple genetic causes are known including dominant mutations in the alpha-synuclein gene; alanine 30 to proline (A30P) and alanine 53 to threonine (A53T), or increased synthesis of the normal a-synuclein. Moreover, α-synuclein protein was identified in Lewy bodies in sporadic PD, further supporting a role for this protein in the pathogenesis of PD.

Additional genes for familial PD have been identified to date: mutations in parkin (PARK2), DJ-1 (PARK7), or PINK1 (PARK6) in familial forms of autosomal recessive parkinsonism and mutations in UCH-L1 (PARK5) and LRRK2/dardarin (PARK8) or genomic triplication of α-synuclein (PARK4) in familial forms of autosomal dominant parkinsonism.

Recent studies suggest that loss of function or gain of a toxic function by the gene products of the above-stated genes is responsible for pathogenesis of both familial and sporadic PD.

In addition, astrocytes have been recently reported to play a role in the progression and in initiating the early neuronal dysfunction and α-synuclein accumulation in astrocytes lead to the cell death of specific neuronal populations in restricted brain regions causing the clinical symptoms of PD. Recent studies indicate that accumulation of a-synuclein in axon-ensheathing oligodendrocytes can also induce neurodegeneration of the associated neurons. Finally, microglia cells have been shown to undergo activated by astrocytes in early staged of PD, which then exert toxic effects on the dopaminergic neurons. Thus, similar to other neurodegenerative disorders the pathogenesis of PD is mediated by pathological cell-cell interactions.

Animal models that harbor genetic lesions that mimic inherited forms of human PD, such as homozygous deletions in parkin or over-expression of α-synuclein have failed to recapitulate the loss of dopaminergic neurons. In contrast, dopaminergic neurons derived from mouse embryonic stem (ES) cells or by iPS of fibroblasts of patients with Parkinson's disease have been shown to represent a better model for studying the pathophysiology of PD.

The disadvantage of this model is in its inability to be translated to clinical applications. In contrast to ESCs and iPS, adult mesenchymal stromal cells (MSCs) derived from bone-marrow, adipose tissue or placenta or MSCs derived from cord cells can be differentiated into neural cells and can be employed to cell therapy of patients with PD.

Novel approaches have been developed for the differentiation of MSCs into dopaminergic neurons and astrocytes and CD34+ cells into microglia cells. Therefore, bone-marrow-derived stem cells from PD patients can be used to understand the molecular mechanisms involved in the cell death of dopaminergic neurons. Specifically, MSCs from patients with different types of PD are differentiated into dopaminergic neurons and astrocytes and CD34+ cells are differentiated into microglia cells. The MSCs and CD34+ cells are examined for mutations in α-synuclein and are analyzed for gene array. The differentiated cells are examined for cell death over a course of one month.

Similarly the effect of astrocytes from PD patients are examined on cell death of the dopaminergic neurons from normal and PD patients in co-culture in the presence and absence of microglia cells that are generated from CD34+ cells from the same patients. In addition to cell death, the neurons are examined for susceptibility to oxidative stress, proteasome inhibition, appearance of insoluble α-synuclein proteins, mitochondria function and ER stress response.

Since dopaminergic neurons, astrocytes and microglia are all potential cellular targets, such a model can provide important information for the development of potential treatment for this disease. In addition, since unmodified and differentiated MSCs to NSCs, neurons and astrocytes have been suggested as a potential cell therapy treatment for this disease, the in vitro model can provide an important input of which cell type, autologous, modified or differentiated or allogeneic, MHC matched or “of the shelf” cord and placenta-derived MSCs may provide a better source for replacement therapy.

Finally, the generation of different PD patient-derived MSCs differentiated to dopaminergic neurons and astrocytes and CD34+ cells differentiated to microglia cells may provide a screening tool for the analysis of small molecules, drugs or gene therapy and silencing approaches.

### Example 3

### Alzheimer's Disease

Alzheimer's disease (AD) is a common chronic neurodegenerative disease and is the most common cause of dementia. The two hallmarks of the disease are senile plaques, which are mainly composed of extracellular deposits of amyloid β and neurofibrillary tangles, which consist of intracellular aggregates of aberrantly phosphorylated tau protein.

The existence of the senile plaques is also associated with an inflammatory response which is considered to play a prominent and early role in AD.

The majority of Alzheimer's disease cases are sporadic, however there are rare cases of dominantly inherited familial forms of Alzheimer's disease that have mutations or a duplication of APP (encodes the amyloid-13 precursor protein), or mutations in the presenilin genes (which encode proteolytic enzymes that cleave APP into amyloid-β). In both types of AD, additional cell types such as astrocytes and microglia have been implicated in the pathogenesis of the disease. In this model MSCs from both familial and sporadic AD patients are differentiated into neurons and astrocytes and CD34+ cells into microglia cells. The cells are analyzed for the levels of amyloid-β, phosphorylated tau, aGSK-3β and synapsin1. The role of astrocytes and microglia from AD patients and controls will delineate the role of these cells in the pathogenesis of the disease and as possible therapeutic targets. Indeed, astrocytes are able to degrade neurotoxic β-amyloid (Aβ) deposits typical for AD both in vitro and in vivo, suggesting that the transplantation of MSC-derived astrocytes may be of therapeutic benefits.

### Example 4

### Rett Syndrome

Rett syndrome (RTT) is an X-chromosome linked autism spectrum disorders that is caused by loss of function of methyl cpG-binding protein 2 (MsCP2). Although earlier studies concluded that the disease is due to the loss of MeCP2 function in neurons, recent studies clearly demonstrated that the loss of MeCP2 in glial cells negatively affected neurons in a non-cell autonomous manner. Moreover, re-expression of MeCP2 in astrocytes in globally MeCP2-deficient mice significantly improved locomotion and anxiety levels, restored respiratory abnormalities to a normal pattern, and greatly prolonged lifespan compared to null mice. In addition, restoration of MeCP2 to the mutant astrocytes exerted a non-cell autonomous positive effect on mutant neurons in vivo, restoring normal dendritic morphology and increasing levels of the excitatory glutamate transporter. Therefore, it appears that glial cells, similar to neurons are integral components of the neuropathology of RTT, and support the idea of administration of glial cells with normal MeCP2 levels as a strategy for improving the associated symptoms of the disease.

In addition to astrocytes, microglia cells have been also implicated in the pathogenesis of the disease by secreting high levels of glutamate which is neuro-cytotoxic.

The in vitro model is consisted of MSC-derived neurons and astrocytes and CD34+-cells derived microglia cells. The survival of the Rett and normal neurons are determined in long-term cultures in the presence and absence of microglia cells and astrocytes. This model can be used for high throughput analysis of novel drugs and for the combination of novel drugs and cell therapy (autologous, allogeneic, universal donors, differentiated cells and growth factor secreting).

### SUMMARY

Recent studies clearly suggest a non-cell autonomous component to the pathogenesis of various neurodegenerative disorders such as ALS, Parkinson's disease, Rett Syndrome and Alzheimer's disease and to psychiatric disease such as schizophrenia. Therefore, studying cell-cell interactions in these diseases is critical for delineating the molecular mechanisms and for designing efficient drug- and cell-based therapies.

Human in vitro models using the relevant cell types are required to complement lower species systems, and thereby confirm, evaluate as well as discover additional mechanisms and targets of direct relevance to human disease.

Additional Disease Specific Models:

Recent studies indicate that there are two common factors that contribute to the pathogenesis of most neurodegenerative disorders: non-cell autonomous component and cell-cell interaction and neuroinflammation.

Therefore in vitro models comprised of MSC-derived neuronal, astrocytic cells and CD34+-derived microglia cells represent the “basic” components of human disease-related models that may be common to many of the neurodegenerative disorders. Moreover, since in many of these diseases, there are additional or secondary mechanisms involved in their pathogenesis, whole gene analysis of the control and differentiated cells may reveal novel therapeutic targets.

### Specific Examples

### 1. Huntington's Disease

The expression of mutant HD gene in glial cells has been shown to exacerbate the neurological symptoms of HD. In addition to the CAG repeat expansion within the coding region of the HD gene, there are defects such as reduced brain cholesterol biosynthesis and ApoE that are manifested mainly in astrocytes, as well as a decreased function of glutamate transporter 1. Microglia activation has been also implicated in the pathogenesis of HD via different pathways.

### 2. Psychiatric Disorders

Schizophrenia is complex genetic disorder with multiple identified risk factors, the most studied is Disrupted in Schizophrenia 1 (DISC1). An accumulating body of evidence point to the significance of neuroinflammation and immunogenetics also in schizophrenia, implicating microglia and astrocytes in the pathogenesis of this disease.

### Advantages of In Vitro MSC Model:

1. Develop personalized disease models—Modeling an “individual” patient disease state in vitro
2. Creating novel neurodegenerative disease models for research, drug screening, toxicology screening and therapeutic testing
3. Personalize regenerative medicine—autologous vs. allogeneic, unmodified vs. differentiated, siRNA, miRNA or growth factor secreting
4. Develop a collection of patient-specific characterized MSCs differentiated to relevant cell types for high throughput drug screening.

### Example 5

### Sequences

TABLE 1

| | Sequence of | Sequence of |
|---|---|---|
| Name | mature miRNA | premiRNA |
| hsa-let-7a | seq id no: 1 | seq id no: 73 |
| | | seq id no: 74 |
| | | seq id no: 75 |
| hsa-let-7b | seq id no: 2 | seq id no: 76 |
| hsa-let-7c | seq id no: 3 | seq id no: 77 |
| hsa-let-7d | seq id no: 4 | seq id no: 78 |
| hsa-let-7e | seq id no: 5 | seq id no: 79 |
| hsa-let-7f | seq id no: 6 | seq id no: 80 |
| hsa-let-7g | seq id no: 7 | seq id no: 81 |
| hsa-let-7i | seq id no: 8 | seq id no: 82 |
| hsa-mir-106a | seq id no: 9 | seq id no: 83 |
| hsa-mir-106b | seq id no: 10 | seq id no: 84 |
| hsa-mir-1294 | seq id no: 11 | seq id no: 85 |
| hsa-mir-1297 | seq id no: 12 | seq id no: 86 |
| hsa-mir-143 | seq id no: 13 | seq id no: 87 |
| hsa-mir-144 | seq id no: 14 | seq id no: 88 |
| hsa-mir-145 | seq id no: 15 | seq id no: 89 |
| hsa-mir-17 | seq id no: 16 | seq id no: 90 |
| miR-181a | seq id no: 17 | seq id no: 91 |
| miR-181a | seq id no: 18 | seq id no: 92 |
| miR-181b | seq id no: 19 | seq id no: 93 |
| miR-181b | seq id no: 20 | seq id no: 94 |
| miR-181c | seq id no: 21 | seq id no: 95 |
| hsa-mir-181d | seq id no: 22 | seq id no: 96 |
| hsa-mir-199a-3p | seq id no: 23 | seq id no: 97 |
| hsa-mir-199b-3p | seq id no: 24 | seq id no: 98 |
| hsa-mir-202 | seq id no: 25 | seq id no: 99 |
| hsa-mir-20a | seq id no: 26 | seq id no: 100 |
| hsa-mir-20b | seq id no: 27 | seq id no: 101 |
| hsa-mir-2113 | seq id no: 28 | seq id no: 102 |
| hsa-mir-25 | seq id no: 29 | seq id no: 103 |
| hsa-mir-26a | seq id no: 30 | seq id no: 104 |
| | seq id no: 31 | seq id no: 105 |
| hsa-mir-26b | seq id no: 32 | seq id no: 106 |
| hsa-mir-29a | seq id no: 33 | seq id no: 107 |
| hsa-mir-29b | seq id no: 34 | seq id no: 108 |
| | | seq id no: 109 |
| hsa-mir-29c | seq id no: 35 | seq id no: 110 |
| hsa-mir-3129-5p | seq id no: 36 | seq id no: 111 |
| hsa-mir-3177-5p | seq id no: 37 | seq id no: 112 |
| hsa-mir-32 | seq id no: 38 | seq id no: 113 |
| hsa-mir-326 | seq id no: 39 | seq id no: 114 |
| hsa-mir-330-5p | seq id no: 40 | seq id no: 115 |
| hsa-mir-363 | seq id no: 41 | seq id no: 116 |
| hsa-mir-3659 | seq id no: 42 | seq id no: 117 |
| hsa-mir-3662 | seq id no: 43 | seq id no: 118 |
| hsa-mir-367 | seq id no: 44 | seq id no: 119 |
| hsa-mir-372 | seq id no: 45 | seq id no: 120 |
| hsa-mir-373 | seq id no: 46 | seq id no: 121 |
| hsa-mir-3927 | seq id no: 47 | seq id no: 122 |
| hsa-mir-4262 | seq id no: 48 | seq id no: 123 |
| hsa-mir-4279 | seq id no: 49 | seq id no: 124 |
| hsa-mir-4458 | seq id no: 50 | seq id no: 125 |
| hsa-mir-4465 | seq id no: 51 | seq id no: 126 |
| hsa-mir-4500 | seq id no: 52 | seq id no: 127 |
| hsa-mir-4658 | seq id no: 53 | seq id no: 128 |
| hsa-mir-4724-3p | seq id no: 54 | seq id no: 129 |
| hsa-mir-4742-3p | seq id no: 55 | seq id no: 130 |
| hsa-mir-4770 | seq id no: 56 | seq id no: 131 |
| hsa-mir-519d | seq id no: 57 | seq id no: 132 |
| hsa-mir-520a-3p | seq id no: 58 | seq id no: 133 |
| hsa-mir-520b | seq id no: 59 | seq id no: 134 |
| hsa-mir-520c-3p | seq id no: 60 | seq id no: 135 |
| hsa-mir-520d-3p | seq id no: 61 | seq id no: 136 |
| hsa-mir-520d-5p | seq id no: 62 | seq id no: 137 |
| hsa-mir-520e | seq id no: 63 | seq id no: 138 |
| hsa-mir-524-5p | seq id no: 64 | seq id no: 139 |
| hsa-mir-642b | seq id no: 65 | seq id no: 140 |
| hsa-mir-656 | seq id no: 66 | seq id no: 141 |
| hsa-mir-767-5p | seq id no: 67 | seq id no: 142 |
| hsa-mir-92a | seq id no: 68 | seq id no: 143 |
| | seq id no: 69 | seq id no: 144 |
| hsa-mir-92b | seq id no: 70 | seq id no: 145 |
| hsa-mir-93 | seq id no: 71 | seq id no: 146 |
| hsa-mir-98 | seq id no: 72 | seq id no: 147 |

TABLE 2

| | Sequence of | Sequence of |
|---|---|---|
| Name | mature | premiRNA |
| hsa-mir-410 | seq id no: 148 | seq id no: 156 |
| hsa-mir-3163 | seq id no: 149 | seq id no: 157 |
| hsa-mir-148a | seq id no: 150 | seq id no: 158 |
| hsa-mir-148b | seq id no: 151 | seq id no: 159 |
| hsa-mir-152 | seq id no: 152 | seq id no: 160 |
| hsa-mir-3121-3p | seq id no: 153 | seq id no: 161 |
| hsa-mir-495 | seq id no: 154 | seq id no: 162 |
| hsa-mir-4680-3p | seq id no: 155 | seq id no: 163 |

TABLE 3

| | Sequence of | | Sequence of |
|---|---|---|---|
| Name | mature | PMIR id | premiRNA |
| miR-92ap | seq id no: 164 | MI0000093 | seq id no: 269 |
| | seq id no: 165 | MI0000094 | seq id no: 270 |
| miR-21 | seq id no: 166 | MI0000077 | seq id no: 271 |
| miR-26a 5P | seq id no: 167 | MI0000083 | seq id no: 272 |
| | seq id no: 168 | MI0000750 | seq id no: 273 |
| miR-18a | seq id no: 169 | MI0000072 | seq id no: 274 |
| miR-124 | seq id no: 170 | MI0000445 | seq id no: 275 |
| | seq id no: 171 | MI0000443 | seq id no: 276 |
| | seq id no: 172 | MI0000444 | seq id no: 277 |
| miR-99a | seq id no: 173 | MI0000101 | seq id no: 278 |
| miR-30c | seq id no: 174 | MI0000736 | seq id no: 279 |
| | | MI0000254 | seq id no: 280 |
| miR-301a 3P | seq id no: 175 | MI0000745 | seq id no: 281 |
| miR-145-50 | seq id no: 176 | MI0000461 | seq id no: 282 |
| miR-143-3p | seq id no: 177 | MI0000459 | seq id no: 283 |
| miR-373 3P | seq id no: 178 | MI0000781 | seq id no: 284 |
| miR-20b | seq id no: 179 | MI0001519 | seq id no: 285 |
| miR-29c 3P | seq id no: 180 | MI0000735 | seq id no: 286 |
| miR-29b 3P | seq id no: 181 | MI0000105 | seq id no: 287 |
| | | MI0000107 | seq id no: 288 |
| miR-143 | | | |
| let-7g | seq id no: 182 | MI0000433 | seq id no: 289 |
| let-7a | seq id no: 183 | MI0000060 | seq id no: 290 |
| | | MI0000061 | seq id no: 291 |
| | | MI0000062 | seq id no: 292 |
| let-7b | seq id no: 184 | MI0000063 | seq id no: 293 |
| miR-98 | seq id no: 185 | MI0000100 | seq id no: 294 |
| miR-30a* | seq id no: 186 | MI0000088 | seq id no: 295 |
| miR-17 | seq id no: 187 | MI0000071 | seq id no: 296 |
| miR-1-1 | seq id no: 188 | MI0000651 | seq id no: 297 |
| miR-1-2 | seq id no: 189 | MI0000437 | seq id no: 298 |
| miR-192 | seq id no: 190 | MI0000234 | seq id no: 299 |
| miR-155 | seq id no: 191 | MI0000681 | seq id no: 300 |
| miR-516-ap a1- | seq id no: 192 | MI0003180 | seq id no: 301 |
| 5p-- | | | |
| a2-3p-- | seq id no: 193 | MI0003181 | seq id no: 302 |
| miR-31 | seq id no: 194 | MI0000089 | seq id no: 303 |
| miR-181a | seq id no: 195 | MI0000289 | seq id no: 304 |
| | seq id no: 196 | MI0000269 | seq id no: 305 |
| miR-181b | seq id no: 197 | MI0000270 | seq id no: 306 |
| | seq id no: 198 | MI0000683 | seq id no: 307 |
| miR-181c | seq id no: 199 | MI0000271 | seq id no: 308 |
| miR-34-c | seq id no: 200 | MI0000743 | seq id no: 309 |
| miR-34b* | seq id no: 201 | MI0000742 | seq id no: 310 |
| miR-103a | seq id no: 202 | MI0000109 | seq id no: 311 |
| | seq id no: 203 | MI0000108 | seq id no: 312 |
| miR-210 | seq id no: 204 | MI0000286 | seq id no: 313 |
| miR-16 | seq id no: 205 | MI0000070 | seq id no: 314 |
| | seq id no: 206 | MI0000115 | seq id no: 315 |
| miR-30a | seq id no: 207 | MI0000088 | seq id no: 316 |
| miR-31 | seq id no: 208 | MI0000089 | seq id no: 317 |
| miR-222 | seq id no: 209 | MI0000299 | seq id no: 318 |
| miR-17 | seq id no: 210 | MI0000071 | seq id no: 319 |
| miR-17* | seq id no: 211 | MI0000071 | seq id no: 320 |
| miR-200b | seq id no: 212 | MI0000342 | seq id no: 321 |
| miR-200c | seq id no: 213 | MI0000650 | seq id no: 322 |
| miR-128 | seq id no: 214 | MI0000447 | seq id no: 323 |
| | | MI0000727 | seq id no: 324 |
| miR-503 | seq id no: 215 | MI0003188 | seq id no: 325 |
| miR-424 | seq id no: 216 | MI0001446 | seq id no: 326 |
| miR-195 | seq id no: 217 | MI0000489 | seq id no: 327 |
| miR-1256 | seq id no: 218 | MI0006390 | seq id no: 328 |
| miR-203a | seq id no: 219 | MI0000283 | seq id no: 329 |
| miR-199 ?? | | | |
| hsa-miR-199a- | seq id no: 220 | MI0000242 | seq id no: 330 |
| 3p_st | | | |
| hsa-miR-199a- | seq id no: 221 | MI0000242 | seq id no: 331 |
| 5p_st | | | |
| hsa-miR-199b- | seq id no: 222 | MI0000282 | seq id no: 332 |
| 3p_st | | | |
| miR-93 | seq id no: 223 | MI0000095 | seq id no: 333 |
| miR-98 | seq id no: 224 | MI0000100 | seq id no: 334 |
| miR-125-a | seq id no: 225 | MI0000469 | seq id no: 335 |
| miR-133a | seq id no: 226 | MI0000450 | seq id no: 336 |
| | | MI0000451 | seq id no: 337 |
| miR-133b | seq id no: 227 | MI0000822 | seq id no: 338 |
| miR-126 | seq id no: 228 | MI0000471 | seq id no: 339 |
| miR-194 | seq id no: 229 | MI0000488 | seq id no: 340 |
| | | MI0000732 | seq id no: 341 |
| miR-346 | seq id no: 230 | MI0000826 | seq id no: 342 |
| miR-15b | seq id no: 231 | MI0000438 | seq id no: 343 |
| miR-338-3p | seq id no: 232 | MI0000814 | seq id no: 344 |
| miR-373 | | | |
| miR-205 | seq id no: 233 | MI0000285 | seq id no: 345 |
| miR-210 | | | |
| miR-125 | | | |
| miR-1226 | seq id no: 234 | MI0006313 | seq id no: 346 |
| miR-708 | seq id no: 235 | MI0005543 | seq id no: 347 |
| miR-449 | seq id no: 236 | MI0001648 | seq id no: 348 |
| miR-422 | seq id no: 237 | MI0001444 | seq id no: 349 |
| miR-340 | seq id no: 238 | MI0000802 | seq id no: 350 |
| miR-605 | seq id no: 239 | MI0003618 | seq id no: 351 |
| miR-522 | seq id no: 240 | MI0003177 | seq id no: 352 |
| miR-663 | seq id no: 241 | MI0003672 | seq id no: 353 |
| miR-130a | seq id no: 242 | MI0000448 | seq id no: 354 |
| miR-130b | seq id no: 243 | MI0000748 | seq id no: 355 |
| miR-942 | seq id no: 244 | MI0005767 | seq id no: 356 |
| miR-572 | seq id no: 245 | MI0003579 | seq id no: 357 |
| miR-520 | | | |
| miR-639 | seq id no: 246 | MI0003654 | seq id no: 358 |
| miR-654 | seq id no: 247 | MI0003676 | seq id no: 359 |
| miR-519 | | | |
| miR-204 | | seq id no: 248 | MI0000284 |
| miR-224 | seq id no: 249 | MI0000301 | seq id no: 360 |
| miR-616 | seq id no: 250 | MI0003629 | seq id no: 361 |
| miR-122 | seq id no: 251 | MI0000442 | seq id no: 362 |
| miR-299 3p- | seq id no: 252 | MI0000744 | seq id no: 363 |
| 5p- | seq id no: 253 | | seq id no: 364 |

| | | |
|---|---|---|
| miR-100 | seq id no: 254 | MI0000102 |

| | | | |
|---|---|---|---|
| miR-138 | seq id no: 255 | MI0000476 | seq id no: 365 |
| miR-140 | seq id no: 256 | MI0000456 | seq id no: 366 |
| miR-375 | seq id no: 257 | MI0000783 | seq id no: 367 |
| miR-217 | seq id no: 258 | MI0000293 | seq id no: 368 |
| miR-302 | | | seq id no: 369 |
| miR-372 | seq id no: 259 | MI0000780 | |
| miR-96 | seq id no: 260 | MI0000098 | seq id no: 370 |
| miR-127-3p | seq id no: 261 | MI0000472 | seq id no: 371 |
| miR-449 | | | seq id no: 372 |
| miR-135b | seq id no: 262 | MI0000810 | |
| miR-101 | seq id no: 263 | MI0000103 | seq id no: 373 |
| | | MI0000739 | seq id no: 374 |
| miR-326 | seq id no: 264 | MI0000808 | seq id no: 375 |
| miR-3245p- | seq id no: 265 | MI0000813 | seq id no: 376 |
| 3p- | seq id no: 266 | MI0000813 | seq id no: 377 |
| miR-335 | seq id no: 267 | MI0000816 | seq id no: 378 |
| miR-141 | seq id no: 268 | MI0000457 | seq id no: 379 |

TABLE 4

| | Sequence of | Sequence of |
|---|---|---|
| Name | mature miRNA | premiRNA |
| miR-1275 | seq id no: 381 | seq id no: 414 |
| miR-891a | seq id no: 382 | seq id no: 415 |
| miR-154 | seq id no: 383 | seq id no: 416 |
| miR-1202 | seq id no: 384 | seq id no: 417 |
| miR-572 | seq id no: 385 | seq id no: 418 |
| miR-935a | seq id no: 386 | seq id no: 419 |
| miR-4317 | seq id no: 387 | seq id no: 420 |
| miR-153 | seq id no: 388 | seq id no: 421 |
| | | seq id no: 422 |
| miR-4288 | seq id no: 389 | seq id no: 423 |
| miR-409-5p | seq id no: 390 | seq id no: 424 |
| miR-193a-5p | seq id no: 391 | seq id no: 425 |
| miR-648 | seq id no: 392 | seq id no: 426 |
| miR-368 | | |
| miR-365 | seq id no: 393 | seq id no: 427 |
| miR-500 | seq id no: 394 | seq id no: 428 |
| miR-491 | seq id no: 395 | seq id no: 429 |
| hsa-miR-199a- | seq id no: 396 | seq id no: 430 |
| 3p_st | | |
| | seq id no: 397 | seq id no: 431 |
| hsa-miR-199a- | seq id no: 398 | seq id no: 432 |
| 5p_st | | |
| | seq id no: 399 | seq id no: 433 |
| miR-2113 | seq id no: 400 | seq id no: 434 |
| miR-372 | seq id no: 401 | seq id no: 435 |
| miR-373 | seq id no: 402 | seq id no: 436 |
| miR-942 | seq id no: 403 | seq id no: 437 |
| miR-1293 | seq id no: 404 | seq id no: 438 |
| miR-18 | seq id no: 405 | seq id no: 439 |
| miR-1182 | seq id no: 406 | seq id no: 440 |
| miR-1185 | seq id no: 407 | seq id no: 441 |
| | | seq id no: 442 |
| miR-1276 | seq id no: 408 | seq id no: 443 |
| miR-193b | seq id no: 409 | seq id no: 444 |
| miR-1238 | seq id no: 410 | seq id no: 445 |
| miR-889 | seq id no: 411 | seq id no: 446 |
| miR-370 | seq id no: 412 | seq id no: 447 |
| miR-548-d1 | seq id no: 413 | seq id no: 448 |

TABLE 5

| | Sequence of |
|---|---|
| Name | mature miRNA |
| hsa-miR-20b | seq id no: 449 |
| hsa-miR-18 | seq id no: 450 |
| hsa-miR-17-5p | seq id no: 451 |
| hsa-miR-141 | seq id no: 452 |
| hsa-miR-302b | seq id no: 453 |
| hsa-miR-101 | seq id no: 454 |
| hsa-miR-126 | seq id no: 455 |
| hsa-miR-146a | seq id no: 456 |
| hsa-miR-146b | seq id no: 457 |
| hsa-miR-26 | seq id no: 458 |
| hsa-miR-29 | seq id no: 459 |
| hsa-miR-132 | seq id no: 460 |
| hsa-miR-9 | seq id no: 461 |
| hsa-miR-146 | seq id no: 462 |
| hsa-miR-10b | seq id no: 463 |
| hsa-miR-222 | seq id no: 464 |
| hsa-miR-193b | seq id no: 465 |
| hsa-miR-221 | seq id no: 466 |
| hsa-miR-135a | seq id no: 467 |
| hsa-miR-149 | seq id no: 468 |
| hsa-miR-199a | seq id no: 469 |
| hsa-miR-302a | seq id no: 470 |
| hsa-miR-302c | seq id no: 471 |
| hsa-miR-302d | seq id no: 472 |
| hsa-miR-369-3p | seq id no: 473 |
| hsa-miR-370 | seq id no: 474 |
| hsa-miR-let7a | seq id no: 475 |
| hsa-miR-let7b | seq id no: 476 |
| hsa-miR-10b | seq id no: 477 |
| hsa-miR-23a | seq id no: 478 |
| hsa-miR-23b | seq id no: 479 |
| hsa-miR-32 | seq id no: 480 |

TABLE 6

| miR designation | seq id no: |
|---|---|
| hsa-miR-20b | seq id no: 449 |
| hsa-miR-18 | seq id no: 450 |
| hsa-miR-17-5p | seq id no: 451 |
| hsa-miR-141 | seq id no: 452 |
| hsa-miR-302b | seq id no: 453 |
| hsa-miR-101 | seq id no: 454 |
| hsa-miR-126 | seq id no: 455 |
| hsa-miR-146a | seq id no: 456 |
| hsa-miR-146b | seq id no: 457 |
| hsa-miR-26 | seq id no: 458 |
| hsa-miR-29 | seq id no: 459 |
| hsa-miR-132 | seq id no: 460 |
| hsa-miR-9 | seq id no: 461 |
| hsa-miR-146 | seq id no: 462 |
| hsa-miR-10b | seq id no: 463 |
| hsa-miR-222 | seq id no: 464 |
| hsa-miR-193b | seq id no: 465 |
| hsa-miR-221 | seq id no: 466 |
| hsa-miR-135a | seq id no: 467 |
| hsa-miR-149 | seq id no: 468 |
| hsa-miR-199a | seq id no: 469 |
| hsa-miR-302a | seq id no: 470 |
| hsa-miR-302c | seq id no: 471 |
| hsa-miR-302d | seq id no: 472 |
| hsa-miR-369-3p | seq id no: 473 |
| hsa-miR-370 | seq id no: 474 |
| hsa-miR-let7a | seq id no: 475 |
| hsa-miR-let7b | seq id no: 476 |
| hsa-miR-10b | seq id no: 477 |
| hsa-miR-23a | seq id no: 478 |
| hsa-miR-23b | seq id no: 479 |
| hsa-miR-32 | seq id no: 480 |
| hsa-miR-371 | seq id no: 481 |
| hsa-miR-134 | seq id no: 482 |
| hsa-miR-219 | seq id no: 483 |
| hsa-miR-154 | seq id no: 484 |
| hsa-miR-155 | seq id no: 485 |
| hsa-miR-33 | seq id no: 486 |
| hsa-miR-127 | seq id no: 487 |
| hsa-miR-132 | seq id no: 488 |
| hsa-miR-137 | seq id no: 489 |
| hsa-miR-142-3p | seq id no: 490 |
| hsa-miR-131a | |
| hsa-miR-125b | seq id no: 491 |
| hsa-miR-153 | seq id no: 492 |
| hsa-miR-181a | seq id no: 493 |
| hsa-miR-123 | |
| hsa-miR-let-7a | seq id no: 494 |
| hsa-miR-let-7b | seq id no: 495 |
| hsa-miR-368 | seq id no: 496 |
| hsa-miR-365-3p | |
| hsa-miR-365-5p | |
| hsa-miR-218 | seq id no: 497 |
| hsa-miR-124 | seq id no: 498 |
| hsa-miR-125a | seq id no: 499 |
| hsa-miR-20a | seq id no: 500 |
| hsa-miR-130a | seq id no: 501 |
| hsa-miR-372 | seq id no: 502 |
| hsa-miR-373 | seq id no: 503 |
| hsa-miR-223 | seq id no: 504 |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

1. A method of qualifying a mesenchymal stem cell (MSC) population, the method comprising:
(a) ex vivo differentiating a population of mesenchymal stem cells originating from the subject towards a first lineage-specific cell, said first lineage-specific cell being associated with a brain disease;
(b) ex vivo differentiating a population of mesenchymal stem cells originating from a healthy subject towards said first lineage-specific cell;
(c) comparing an effect of said first lineage specific cell derived from said subject with an effect of said first lineage specific cell derived from said healthy subject on a second lineage specific cell associated with the brain disease, wherein a difference in said effect above or below a predetermined level is indicative of a qualification of a mesenchymal stem cell population.

2. The method of claim 1, wherein the qualifying is to ascertain whether the MSC population is suitable for autologous cell replacement therapy for the treatment of the brain disease of a subject.

3. (canceled)

4. The method of claim 1, wherein said second lineage specific cell has been ex vivo differentiated from a population of mesenchymal stem cells.

5-8. (canceled)

9. The method of claim 1, further comprising analyzing for a genetic aberration of said MSCs originating from the subject, said genetic aberrations being associated with a brain disease; or
comparing an effect of a third lineage specific cell derived from said subject with an effect of said third lineage specific cell derived from said healthy subject on said first and/or said second lineage specific cell.

10. A method of qualifying a population of mesenchymal stem cells (MSCs), the method comprising analyzing a function and/or morphology of a lineage-specific cell differentiated from the population of the MSCs, wherein an alteration in said function or morphology of said lineage-specific cell compared to a control lineage specific cell is indicative of a qualification of the MSCs.

11. The method of claim 10, wherein the population of MSCs is derived from a subject having a brain disease.

12. The method of claim 11, wherein said qualifying is to ascertain whether the MSC population is suitable for autologous cell replacement therapy for the treatment of the brain disease of a subject.

13. The method of claim 10, further comprising analyzing for a genetic aberration of said MSCs originating from the subject, said genetic aberrations being associated with a brain disease.

14. The method of claim 10, wherein said function of said lineage-specific cell comprises a response to a microglia cell.

15-17. (canceled)

18. The method of claim 11, wherein:
(i) when the brain disease is Amyotrophic Lateral Sclerosis (ALS), said lineage specific cells are selected from the group consisting of motor neurons, skeletal muscle and astrocytes;
(ii) when the brain disease is Parkinson's disease, said lineage specific cells are selected from the group consisting of astrocytes and dopaminergic neurons; and
(iii) when the brain disease is Rett syndrome, said lineage specific cells are selected from the group consisting of astrocytes and neurons.

19-20. (canceled)

21. A method of selecting an agent for the treatment of a brain disease, the method comprising analyzing an ability of a population of MSCs isolated from a patient with a brain disorder to differentiate towards a lineage-specific cell associated with said brain disorder in a presence and absence of said agent, wherein an improvement of a function or morphology of said lineage specific cell as compared to an identical lineage specific cell derived from a control population of MSCs in said presence of said agent is indicative that the agent may be used for the treatment of the brain disease.

22-35. (canceled)


SEQUENCE LISTING (504 sequences)

SEQ ID NO: 1 - RNA, 22 nt, Homo sapiens
ugagguaguagguuguauaguu

SEQ ID NO: 2 - RNA, 22 nt, Homo sapiens
ugagguaguagguugugugguu

SEQ ID NO: 3 - RNA, 22 nt, Homo sapiens
ugagguaguagguuguaugguu

SEQ ID NO: 4 - RNA, 22 nt, Homo sapiens
agagguaguagguugcauaguu

SEQ ID NO: 5 - RNA, 22 nt, Homo sapiens
ugagguaggagguuguauaguu

SEQ ID NO: 6 - RNA, 22 nt, Homo sapiens
ugagguaguagauuguauaguu

SEQ ID NO: 7 - RNA, 22 nt, Homo sapiens
ugagguaguaguuuguacaguu

SEQ ID NO: 8 - RNA, 22 nt, Homo sapiens
ugagguaguaguuugugcuguu

SEQ ID NO: 9 - RNA, 23 nt, Homo sapiens
aaaagugcuuacagugcagguag

SEQ ID NO: 10 - RNA, 21 nt, Homo sapiens
uaaagugcugacagugcagau

SEQ ID NO: 11 - RNA, 22 nt, Homo sapiens
ugugagguuggcauuguugucu

SEQ ID NO: 12 - RNA, 17 nt, Homo sapiens
uucaaguaauucaggug

SEQ ID NO: 13 - RNA, 22 nt, Homo sapiens
ggugcagugcugcaucucuggu

SEQ ID NO: 14 - RNA, 20 nt, Homo sapiens
uacaguauagaugauguacu

SEQ ID NO: 15 - RNA, 23 nt, Homo sapiens
guccaguuuucccaggaaucccu

SEQ ID NO: 16 - RNA, 23 nt, Homo sapiens
caaagugcuuacagugcagguag

SEQ ID NO: 17 - RNA, 23 nt, Homo sapiens
aacauucaacgcugucggugagu

SEQ ID NO: 18 - RNA, 23 nt, Homo sapiens
aacauucaacgcugucggugagu

SEQ ID NO: 19 - RNA, 23 nt, Homo sapiens
aacauucauugcugucggugggu

SEQ ID NO: 20 - RNA, 23 nt, Homo sapiens
aacauucauugcugucggugggu

SEQ ID NO: 21 - RNA, 22 nt, Homo sapiens
aacauucaaccugucggugagu

SEQ ID NO: 22 - RNA, 23 nt, Homo sapiens
aacauucauuguugucggugggu

SEQ ID NO: 23 - RNA, 22 nt, Homo sapiens
acaguagucugcacauugguua

SEQ ID NO: 24 - RNA, 22 nt, Homo sapiens
acaguagucugcacauugguua

SEQ ID NO: 25 - RNA, 20 nt, Homo sapiens
agagguauagggcaugggaa

SEQ ID NO: 26 - RNA, 23 nt, Homo sapiens
uaaagugcuuauagugcagguag

SEQ ID NO: 27 - RNA, 23 nt, Homo sapiens
caaagugcucauagugcagguag

SEQ ID NO: 28 - RNA, 21 nt, Homo sapiens
auuugugcuuggcucugucac

SEQ ID NO: 29 - RNA, 22 nt, Homo sapiens
cauugcacuugucucggucuga

SEQ ID NO: 30 - RNA, 22 nt, Homo sapiens
uucaaguaauccaggauaggcu

SEQ ID NO: 31 - RNA, 22 nt, Homo sapiens
uucaaguaauccaggauaggcu

SEQ ID NO: 32 - RNA, 21 nt, Homo sapiens
uucaaguaauucaggauaggu

SEQ ID NO: 33 - RNA, 22 nt, Homo sapiens
uagcaccaucugaaaucgguua

SEQ ID NO: 34 - RNA, 23 nt, Homo sapiens
uagcaccauuugaaaucaguguu

SEQ ID NO: 35 - RNA, 22 nt, Homo sapiens
uagcaccauuugaaaucgguua

SEQ ID NO: 36 - RNA, 22 nt, Homo sapiens
gcaguaguguagagauugguuu

SEQ ID NO: 37 - RNA, 23 nt, Homo sapiens
uguguacacacgugccaggcgcu

SEQ ID NO: 38 - RNA, 22 nt, Homo sapiens
uauugcacauuacuaaguugca

SEQ ID NO: 39 - RNA, 20 nt, Homo sapiens
ccucugggcccuuccuccag

SEQ ID NO: 40 - RNA, 23 nt, Homo sapiens
gcaaagcacacggccugcagaga

SEQ ID NO: 41 - RNA, 22 nt, Homo sapiens
aauugcacgguauccaucugua

SEQ ID NO: 42 - RNA, 21 nt, Homo sapiens
ugaguguugucuacgagggca

SEQ ID NO: 43 - RNA, 24 nt, Homo sapiens
gaaaaugaugaguagugacugaug

SEQ ID NO: 44 - RNA, 22 nt, Homo sapiens
aauugcacuuuagcaaugguga

SEQ ID NO: 45 - RNA, 23 nt, Homo sapiens
aaagugcugcgacauuugagcgu

SEQ ID NO: 46 - RNA, 23 nt, Homo sapiens
gaagugcuucgauuuuggggugu

SEQ ID NO: 47 - RNA, 22 nt, Homo sapiens
cagguagauauuugauaggcau

SEQ ID NO: 48 - RNA, 17 nt, Homo sapiens
gacauucagacuaccug

SEQ ID NO: 49 - RNA, 16 nt, Homo sapiens
cucuccucccggcuuc

SEQ ID NO: 50 - RNA, 19 nt, Homo sapiens
agagguagguguggaagaa

SEQ ID NO: 51 - RNA, 22 nt, Homo sapiens
cucaaguagucugaccagggga

SEQ ID NO: 52 - RNA, 17 nt, Homo sapiens
ugagguaguaguuucuu

SEQ ID NO: 53 - RNA, 23 nt, Homo sapiens
gugaguguggauccuggaggaau

SEQ ID NO: 54 - RNA, 21 nt, Homo sapiens
guaccuucugguucagcuagu

SEQ ID NO: 55 - RNA, 23 nt, Homo sapiens
ucuguauucuccuuugccugcag

SEQ ID NO: 56 - RNA, 18 nt, Homo sapiens
ugagaugacacuguagcu

SEQ ID NO: 57 - RNA, 22 nt, Homo sapiens
caaagugccucccuuuagagug

SEQ ID NO: 58 - RNA, 22 nt, Homo sapiens
aaagugcuucccuuuggacugu

SEQ ID NO: 59 - RNA, 21 nt, Homo sapiens
aaagugcuuccuuuuagaggg

SEQ ID NO: 60 - RNA, 22 nt, Homo sapiens
aaagugcuuccuuuuagagggu

SEQ ID NO: 61 - RNA, 22 nt, Homo sapiens
aaagugcuucucuuuggugggu

SEQ ID NO: 62 - RNA, 20 nt, Homo sapiens
cuacaaagggaagcccuuuc

SEQ ID NO: 63 - RNA, 21 nt, Homo sapiens
aaagugcuuccuuuuugaggg

SEQ ID NO: 64 - RNA, 22 nt, Homo sapiens
cuacaaagggaagcacuuucuc

SEQ ID NO: 65 - RNA, 22 nt, Homo sapiens
agacacauuuggagagggaccc

SEQ ID NO: 66 - RNA, 21 nt, Homo sapiens
aauauuauacagucaaccucu

SEQ ID NO: 67 - RNA, 23 nt, Homo sapiens
ugcaccaugguugucugagcaug

SEQ ID NO: 68 - RNA, 22 nt, Homo sapiens
uauugcacuugucccggccugu

SEQ ID NO: 69 - RNA, 22 nt, Homo sapiens
uauugcacuugucccggccugu

SEQ ID NO: 70 - RNA, 22 nt, Homo sapiens
uauugcacucgucccggccucc

SEQ ID NO: 71 - RNA, 23 nt, Homo sapiens
caaagugcuguucgugcagguag

SEQ ID NO: 72 - RNA, 22 nt, Homo sapiens
ugagguaguaaguuguauuguu

SEQ ID NO: 73 - RNA, 80 nt, Homo sapiens
ugggaugagguaguagguuguauaguuuuagggucacacccaccacugggagauaacuauacaaucuacugucuuuccua

SEQ ID NO: 74 - RNA, 72 nt, Homo sapiens
agguugagguaguagguuguauaguuuagaauuacaucaagggagauaacuguacagccuccuagcuuuccu

SEQ ID NO: 75 - RNA, 74 nt, Homo sapiens
gggugagguaguagguuguauaguuuggggcucugcccugcuaugggauaacuauacaaucuacugucuuuccu

SEQ ID NO: 76 - RNA, 83 nt, Homo sapiens
cggggugagguaguagguugugugguuucagggcagugauguugccccucggaagauaacuauacaaccuacugccuucccug

SEQ ID NO: 77 - RNA, 84 nt, Homo sapiens
gcauccggguugagguaguagguuguaugguuuagaguuacacccugggaguuaacuguacaaccuucuagcuuuccuuggagc

SEQ ID NO: 78 - RNA, 87 nt, Homo sapiens
ccuaggaagagguaguagguugcauaguuuuagggcagggauuuugcccacaaggagguaacuauacgaccugcugccuuucuuagg

SEQ ID NO: 79 - RNA, 79 nt, Homo sapiens
cccgggcugagguaggagguuguauaguugaggaggacacccaaggagaucacuauacggccuccuagcuuuccccagg

SEQ ID NO: 80 - RNA, 87 nt, Homo sapiens
ucagagugagguaguagauuguauaguugugggguagugauuuuacccuguucaggagauaacuauacaaucuauugccuucccuga

SEQ ID NO: 81 - RNA, 84 nt, Homo sapiens
aggcugagguaguaguuuguacaguuugagggucuaugauaccacccgguacaggagauaacuguacaggccacugccuugcca

SEQ ID NO: 82 - RNA, 84 nt, Homo sapiens
cuggcugagguaguaguuugugcuguuggucggguugugacauugcccgcuguggagauaacugcgcaagcuacugccuugcua

SEQ ID NO: 83 - RNA, 81 nt, Homo sapiens
ccuuggccauguaaaagugcuuacagugcagguagcuuuuugagaucuacugcaauguaagcacuucuuacauuaccaugg

SEQ ID NO: 84 - RNA, 82 nt, Homo sapiens
ccugccggggcuaaagugcugacagugcagauagugguccucuccgugcuaccgcacuguggguacuugcugcuccagcagg

SEQ ID NO: 85 - RNA, 142 nt, Homo sapiens
caccuaaugugugccaagaucuguucauuuaugaucucaccgaguccugugagguuggcauuguugucuggcauugucugauauacaacagugccaaccucacaggacucagugaggugaaacugaggauuaggaaggugua

SEQ ID NO: 86 - RNA, 77 nt, Homo sapiens
uguuuaucucuaggguugaucuauuagaauuacuuaucugagccaaaguaauucaaguaauucagguguagugaaac

SEQ ID NO: 87 - RNA, 106 nt, Homo sapiens
gcgcagcgcccugucucccagccugaggugcagugcugcaucucuggucaguugggagucugagaugaagcacuguagcucaggaagagagaaguuguucugcagc

SEQ ID NO: 88 - RNA, 86 nt, Homo sapiens
uggggcccuggcugggauaucaucauauacuguaaguuugcgaugagacacuacaguauagaugauguacuaguccgggcaccccc

SEQ ID NO: 89 - RNA, 88 nt, Homo sapiens
caccuuguccucacgguccaguuuucccaggaaucccuuagaugcuaagauggggauuccuggaaauacuguucuugaggucaugguu

SEQ ID NO: 90 - RNA, 84 nt, Homo sapiens
gucagaauaaugucaaagugcuuacagugcagguagugauaugugcaucuacugcagugaaggcacuuguagcauuauggugac

SEQ ID NO: 91 - RNA, 110 nt, Homo sapiens
ugaguuuugagguugcuucagugaacauucaacgcugucggugaguuuggaauuaaaaucaaaaccaucgaccguugauuguacccuauggcuaaccaucaucuacucca

SEQ ID NO: 92 - RNA, 110 nt, Homo sapiens
agaagggcuaucaggccagccuucagaggacuccaaggaacauucaacgcugucggugaguuugggauuugaaaaaaccacugaccguugacuguaccuugggguccuua

SEQ ID NO: 93 - RNA, 110 nt, Homo sapiens
ccugugcagagauuauuuuuuaaaaggucacaaucaacauucauugcugucgguggguugaacuguguggacaagcucacugaacaaugaaugcaacuguggccccgcuu

SEQ ID NO: 94 - RNA, 89 nt, Homo sapiens
cugauggcugcacucaacauucauugcugucgguggguuugagucugaaucaacucacugaucaaugaaugcaaacugcggaccaaaca

SEQ ID NO: 95 - RNA, 110 nt, Homo sapiens
cggaaaauuugccaaggguuugggggaacauucaaccugucggugaguuugggcagcucaggcaaaccaucgaccguugaguggacccugaggccuggaauugccauccu

SEQ ID NO: 96 - RNA, 137 nt, Homo sapiens
guccccuccccuaggccacagccgaggucacaaucaacauucauuguugucgguggguugugaggacugaggccagacccaccgggggaugaaugucacuguggcugggccagacacggcuuaaggggaauggggac

SEQ ID NO: 97 - RNA, 71 nt, Homo sapiens
gccaacccaguguucagacuaccuguucaggaggcucucaauguguacaguagucugcacauugguuaggc

SEQ ID NO: 98 - RNA, 110 nt, Homo sapiens
ccagaggacaccuccacuccgucuacccaguguuuagacuaucuguucaggacucccaaauuguacaguagucugcacauugguuaggcugggcuggguuagacccucgg

SEQ ID NO: 99 - RNA, 110 nt, Homo sapiens
cgccucagagccgcccgccguuccuuuuuccuaugcauauacuucuuugaggaucuggccuaaagagguauagggcaugggaaaacggggcggucggguccuccccagcg

SEQ ID NO: 100 - RNA, 71 nt, Homo sapiens
guagcacuaaagugcuuauagugcagguaguguuuaguuaucuacugcauuaugagcacuuaaaguacugc

SEQ ID NO: 101 - RNA, 69 nt, Homo sapiens
aguaccaaagugcucauagugcagguaguuuuggcaugacucuacuguaguaugggcacuuccaguacu

SEQ ID NO: 102 - RNA, 91 nt, Homo sapiens
uuuucaaagcaaugugugacagguacagggacaaaucccguuaauaaguaagaggauuugugcuuggcucugucacaugccacuuugaaaa

SEQ ID NO: 103 - RNA, 84 nt, Homo sapiens
ggccaguguugagaggcggagacuugggcaauugcuggacgcugcccugggcauugcacuugucucggucugacagugccggcc

SEQ ID NO: 104 - RNA, 77 nt, Homo sapiens
guggccucguucaaguaauccaggauaggcugugcaggucccaaugggccuauucuugguuacuugcacggggacgc

SEQ ID NO: 105 - RNA, 84 nt, Homo sapiens
ggcuguggcuggauucaaguaauccaggauaggcuguuuccaucugugaggccuauucuugauuacuuguuucuggaggcagcu

SEQ ID NO: 106 - RNA, 77 nt, Homo sapiens
ccgggacccaguucaaguaauucaggauagguugugugcuguccagccuguucuccauuacuuggcucggggaccgg

SEQ ID NO: 107 - RNA, 64 nt, Homo sapiens
augacugauuucuuuugguguucagagucaauauaauuuucuagcaccaucugaaaucgguuau

SEQ ID NO: 108 - RNA, 81 nt, Homo sapiens
cuucaggaagcugguuucauauggugguuuagauuuaaauagugauugucuagcaccauuugaaaucaguguucuuggggg

SEQ ID NO: 109 - RNA, 81 nt, Homo sapiens
cuucuggaagcugguuucacaugguggcuuagauuuuuccaucuuuguaucuagcaccauuugaaaucaguguuuuaggag

SEQ ID NO: 110 - RNA, 88 nt, Homo sapiens
aucucuuacacaggcugaccgauuucuccugguguucagagucuguuuuugucuagcaccauuugaaaucgguuaugauguaggggga

SEQ ID NO: 111 - RNA, 76 nt, Homo sapiens
guacuugggcaguaguguagagauugguuugccuguuaaugaauucaaacuaaucucuacacugcugcccaagagc

SEQ ID NO: 112 - RNA, 82 nt, Homo sapiens
ccacgugccauguguacacacgugccaggcgcugucuugagacauucgcgcagugcacggcacuggggacacguggcacugg

SEQ ID NO: 113 - RNA, 70 nt, Homo sapiens
ggagauauugcacauuacuaaguugcauguugucacggccucaaugcaauuuagugugugugauauuuuc

SEQ ID NO: 114 - RNA, 95 nt, Homo sapiens
cucaucugucuguugggcuggaggcagggccuuugugaaggcggguggugcucagaucgccucugggcccuuccuccagccccgaggcggauuca

SEQ ID NO: 115 - RNA, 94 nt, Homo sapiens
cuuuggcgaucacugccucucugggccugugucuuaggcucugcaagaucaaccgagcaaagcacacggccugcagagaggcagcgcucugccc

SEQ ID NO: 116 - RNA, 75 nt, Homo sapiens
uguugucggguggaucacgaugcaauuuugaugaguaucauaggagaaaaauugcacgguauccaucuguaaacc

SEQ ID NO: 117 - RNA, 99 nt, Homo sapiens
ucuacaagcagauacaaggaugcccuuguacacaacacacgugcugcuuguauagacaugaguguugucuacgagggcauccuugugucugugugugug

SEQ ID NO: 118 - RNA, 95 nt, Homo sapiens
uguguuuuccucaacgcucacaguuacacuucuuacucucaauccauucauauugaaaaugaugaguagugacugaugaagcacaaaucagccaa

SEQ ID NO: 119 - RNA, 68 nt, Homo sapiens
ccauuacuguugcuaauaugcaacucuguugaauauaaauuggaauugcacuuuagcaauggugaugg

SEQ ID NO: 120 - RNA, 67 nt, Homo sapiens
gugggccucaaauguggagcacuauucugauguccaaguggaaagugcugcgacauuugagcgucac

SEQ ID NO: 121 - RNA, 69 nt, Homo sapiens
gggauacucaaaaugggggcgcuuuccuuuuugucuguacugggaagugcuucgauuuugggguguccc

SEQ ID NO: 122 - RNA, 71 nt, Homo sapiens
ugccaaugccuaucacauaucugccuguccuaugacaaacauggcagguagauauuugauaggcauuggca

SEQ ID NO: 123 - RNA, 54 nt, Homo sapiens
gaaagcugcaggugcugauguuggggggacauucagacuaccugcagcagagcc

SEQ ID NO: 124 - RNA, 58 nt, Homo sapiens
ugcucuguggagcugaggagcagauucucucucucuccucccggcuucaccuccugag

SEQ ID NO: 125 - RNA, 75 nt, Homo sapiens
gagcgcacagagguagguguggaagaaagugaaacacuauuuuagguuuuaguuacacucugcuguggugugcug

SEQ ID NO: 126 - RNA, 70 nt, Homo sapiens
cauguguccccuggcacgcuauuugagguuuacuauggaaccucaaguagucugaccaggggacacauga

SEQ ID NO: 127 - RNA, 76 nt, Homo sapiens
caggagagaaaguacugcccagaagcuaaaguguagaucaaacgcauaauggcugagguaguaguuucuugaacuu

SEQ ID NO: 128 - RNA, 65 nt, Homo sapiens
gcugcccuucacucagagcaucuacacccacuaccggugaguguggauccuggaggaaucguggc

SEQ ID NO: 129 - RNA, 89 nt, Homo sapiens
acgcaaaaugaacugaaccaggagugagcuucguguacauuaucuauuagaaaaugaaguaccuucugguucagcuagucccugugcgu

SEQ ID NO: 130 - RNA, 85 nt, Homo sapiens
ucaggcaaagggauauuuacagauacuuuuuaaaauuuguuugaguugaggcagauuaaauaucuguauucuccuuugccugcag

SEQ ID NO: 131 - RNA, 58 nt, Homo sapiens
gaguuauggggucaucuauccuucccuuggaaaaugaucugagaugacacuguagcuc

SEQ ID NO: 132 - RNA, 88 nt, Homo sapiens
ucccaugcugugacccuccaaagggaagcgcuuucuguuuguuuucucuuaaacaaagugccucccuuuagaguguuaccguuuggga

SEQ ID NO: 133 - RNA, 85 nt, Homo sapiens
cucaggcugugacccuccagagggaaguacuuucuguugucugagagaaaagaaagugcuucccuuuggacuguuucgguuugag

SEQ ID NO: 134 - RNA, 61 nt, Homo sapiens
cccucuacagggaagcgcuuucuguugucugaaagaaaagaaagugcuuccuuuuagaggg

SEQ ID NO: 135 - RNA, 87 nt, Homo sapiens
ucucaggcugucguccucuagagggaagcacuuucuguugucugaaagaaaagaaagugcuuccuuuuagaggguuaccguuugaga

SEQ ID NO: 136 - RNA, 87 nt, Homo sapiens
ucucaagcugugagucuacaaagggaagcccuuucuguugucuaaaagaaaagaaagugcuucucuuugguggguuacgguuugaga

SEQ ID NO: 137 - RNA, 87 nt, Homo sapiens; identical to SEQ ID NO: 136

SEQ ID NO: 138 - RNA, 87 nt, Homo sapiens
ucuccugcugugacccucaagauggaagcaguuucuguugucugaaaggaaagaaagugcuuccuuuuugaggguuacuguuugaga

SEQ ID NO: 139 - RNA, 87 nt, Homo sapiens
ucucaugcugugacccuacaaagggaagcacuuucucuuguccaaaggaaaagaaggcgcuucccuuuggaguguuacgguuugaga

SEQ ID NO: 140 - RNA, 77 nt, Homo sapiens
gaguugggagguucccucuccaaaugugucuugaucccccaccccaagacacauuuggagagggacccucccaacuc

SEQ ID NO: 141 - RNA, 78 nt, Homo sapiens
cugaaauagguugccugugagguguucacuuucuauaugaugaauauuauacagucaaccucuuuccgauaucgaauc

SEQ ID NO: 142 - RNA, 109 nt, Homo sapiens
gcuuuuauauuguagguuuuugcucaugcaccaugguugucugagcaugcagcaugcuugucugcucauaccccaugguuucugagcaggaaccuucauugucuacugc

SEQ ID NO: 143 - RNA, 78 nt, Homo sapiens
cuuucuacacagguugggaucgguugcaaugcuguguuucuguaugguauugcacuugucccggccuguugaguuugg

SEQ ID NO: 144 - RNA, 75 nt, Homo sapiens
ucaucccuggguggggauuuguugcauuacuuguguucuauauaaaguauugcacuugucccggccuguggaaga

SEQ ID NO: 145 - RNA, 96 nt, Homo sapiens
cgggccccgggcgggcgggagggacgggacgcggugcaguguuguuuuuucccccgccaauauugcacucgucccggccuccggcccccccggccc

SEQ ID NO: 146 - RNA, 80 nt, Homo sapiens
cugggggcuccaaagugcuguucgugcagguagugugauuacccaaccuacugcugagcuagcacuucccgagcccccgg

SEQ ID NO: 147 - RNA, 119 nt, Homo sapiens
aggauucugcucaugccagggugagguaguaaguuguauuguugugggguagggauauuaggccccaauuagaagauaacuauacaacuuacuacuuucccugguguguggcauauuca

SEQ ID NO: 148 - RNA, 21 nt, Homo sapiens
aauauaacacagauggccugu

SEQ ID NO: 149 - RNA, 22 nt, Homo sapiens
uauaaaaugagggcaguaagac

SEQ ID NO: 150 - RNA, 22 nt, Homo sapiens
ucagugcacuacagaacuuugu

SEQ ID NO: 151 - RNA, 22 nt, Homo sapiens
ucagugcaucacagaacuuugu

SEQ ID NO: 152 - RNA, 21 nt, Homo sapiens
ucagugcaugacagaacuugg

SEQ ID NO: 153 - RNA, 22 nt, Homo sapiens
uaaauagaguaggcaaaggaca

SEQ ID NO: 154 - RNA, 22 nt, Homo sapiens
aaacaaacauggugcacuucuu

SEQ ID NO: 155 - RNA, 21 nt, Homo sapiens
ucugaauuguaagaguuguua

SEQ ID NO: 156 - RNA, 80 nt, Homo sapiens
gguaccugagaagagguugucugugaugaguucgcuuuuauuaaugacgaauauaacacagauggccuguuuucaguacc

SEQ ID NO: 157 - RNA, 73 nt, Homo sapiens
uuccucaucuauaaaaugagggcaguaagaccuuccuuccuugucuuacuacccccauuuuauagaugaggaa

SEQ ID NO: 158 - RNA, 68 nt, Homo sapiens
gaggcaaaguucugagacacuccgacucugaguaugauagaagucagugcacuacagaacuuugucuc

SEQ ID NO: 159 - RNA, 99 nt, Homo sapiens
caagcacgauuagcauuugaggugaaguucuguuauacacucaggcuguggcucucugaaagucagugcaucacagaacuuugucucgaaagcuuucua

SEQ ID NO: 160 - RNA, 87 nt, Homo sapiens
ugucccccccggcccagguucugugauacacuccgacucgggcucuggagcagucagugcaugacagaacuugggcccggaaggacc

SEQ ID NO: 161 - RNA, 77 nt, Homo sapiens
aaaugguuauguccuuugccuauucuauuuaagacacccuguaccuuaaauagaguaggcaaaggacagaaacauuu

SEQ ID NO: 162 - RNA, 82 nt, Homo sapiens
ugguaccugaaaagaaguugcccauguuauuuucgcuuuauaugugacgaaacaaacauggugcacuucuuuuucgguauca

SEQ ID NO: 163 - RNA, 66 nt, Homo sapiens
uauaagaacucuugcagucuuagauguuauaaaaauauauaucugaauuguaagaguuguuagcac

SEQ ID NO: 164 - RNA, 22 nt, Homo sapiens
uauugcacuugucccggccugu

SEQ ID NO: 165 - RNA, 22 nt, Homo sapiens
uauugcacuugucccggccugu

SEQ ID NO: 166 - RNA, 22 nt, Homo sapiens
uagcuuaucagacugauguuga

SEQ ID NO: 167 - RNA, 22 nt, Homo sapiens
uucaaguaauccaggauaggcu

SEQ ID NO: 168 - RNA, 22 nt, Homo sapiens
uucaaguaauccaggauaggcu

SEQ ID NO: 169 - RNA, 23 nt, Homo sapiens
uaaggugcaucuagugcagauag

SEQ ID NO: 170 - RNA, 20 nt, Homo sapiens
uaaggcacgcggugaaugcc

SEQ ID NO: 171 - RNA, 20 nt, Homo sapiens
uaaggcacgcggugaaugcc

SEQ ID NO: 172 - RNA, 20 nt, Homo sapiens
uaaggcacgcggugaaugcc

SEQ ID NO: 173 - RNA, 22 nt, Homo sapiens
aacccguagauccgaucuugug

SEQ ID NO: 174 - RNA, 23 nt, Homo sapiens
uguaaacauccuacacucucagc

SEQ ID NO: 175 - RNA, 23 nt, Homo sapiens
cagugcaauaguauugucaaagc

SEQ ID NO: 176 - RNA, 23 nt, Homo sapiens
guccaguuuucccaggaaucccu

SEQ ID NO: 177 - RNA, 22 nt, Homo sapiens
ggugcagugcugcaucucuggu

SEQ ID NO: 178 - RNA, 23 nt, Homo sapiens
gaagugcuucgauuuuggggugu

SEQ ID NO: 179 - RNA, 23 nt, Homo sapiens
caaagugcucauagugcagguag

SEQ ID NO: 180 - RNA, 22 nt, Homo sapiens
uagcaccauuugaaaucgguua

SEQ ID NO: 181 - RNA, 23 nt, Homo sapiens
uagcaccauuugaaaucaguguu

SEQ ID NO: 182 - RNA, 22 nt, Homo sapiens
ugagguaguaguuuguacaguu

SEQ ID NO: 183 - RNA, 22 nt, Homo sapiens
ugagguaguagguuguauaguu

SEQ ID NO: 184 - RNA, 22 nt, Homo sapiens
ugagguaguagguugugugguu

SEQ ID NO: 185 - RNA, 22 nt, Homo sapiens
ugagguaguaaguuguauuguu

SEQ ID NO: 186 - RNA, 22 nt, Homo sapiens
cuuucagucggauguuugcagc

SEQ ID NO: 187 - RNA, 23 nt, Homo sapiens
caaagugcuuacagugcagguag

SEQ ID NO: 188 - RNA, 22 nt, Homo sapiens
uggaauguaaagaaguauguau

SEQ ID NO: 189 - RNA, 22 nt, Homo sapiens
uggaauguaaagaaguauguau

SEQ ID NO: 190 - RNA, 21 nt, Homo sapiens
cugaccuaugaauugacagcc

SEQ ID NO: 191 - RNA, 23 nt, Homo sapiens
uuaaugcuaaucgugauaggggu

SEQ ID NO: 192 - RNA, 23 nt, Homo sapiens
uucucgaggaaagaagcacuuuc

SEQ ID NO: 193 - RNA, 18 nt, Homo sapiens
ugcuuccuuucagagggu

SEQ ID NO: 194 - RNA, 21 nt, Homo sapiens
aggcaagaugcuggcauagcu

SEQ ID NO: 195 - RNA, 23 nt, Homo sapiens
aacauucaacgcugucggugagu

SEQ ID NO: 196 - RNA, 23 nt, Homo sapiens
aacauucaacgcugucggugagu

SEQ ID NO: 197 - RNA, 23 nt, Homo sapiens
aacauucauugcugucggugggu

SEQ ID NO: 198 - RNA, 23 nt, Homo sapiens
aacauucauugcugucggugggu

SEQ ID NO: 199 - RNA, 22 nt, Homo sapiens
aacauucaaccugucggugagu

SEQ ID NO: 200 - RNA, 23 nt, Homo sapiens
aggcaguguaguuagcugauugc

SEQ ID NO: 201 - RNA, 23 nt, Homo sapiens
uaggcagugucauuagcugauug

SEQ ID NO: 202 - RNA, 23 nt, Homo sapiens
agcagcauuguacagggcuauga

SEQ ID NO: 203 - RNA, 23 nt, Homo sapiens
agcagcauuguacagggcuauga

SEQ ID NO: 204 - RNA, 22 nt, Homo sapiens
cugugcgugugacagcggcuga

SEQ ID NO: 205 - RNA, 22 nt, Homo sapiens
uagcagcacguaaauauuggcg

SEQ ID NO: 206 - RNA, 22 nt, Homo sapiens
uagcagcacguaaauauuggcg

SEQ ID NO: 207 - RNA, 22 nt, Homo sapiens
uguaaacauccucgacuggaag

SEQ ID NO: 208 - RNA, 21 nt, Homo sapiens
aggcaagaugcuggcauagcu

SEQ ID NO: 209 - RNA, 21 nt, Homo sapiens
agcuacaucuggcuacugggu

SEQ ID NO: 210 - RNA, 23 nt, Homo sapiens
caaagugcuuacagugcagguag

SEQ ID NO: 211 - RNA, 22 nt, Homo sapiens
acugcagugaaggcacuuguag

SEQ ID NO: 212 - RNA, 22 nt, Homo sapiens
uaauacugccugguaaugauga

SEQ ID NO: 213 - RNA, 23 nt, Homo sapiens
uaauacugccggguaaugaugga

SEQ ID NO: 214 - RNA, 21 nt, Homo sapiens
ucacagugaaccggucucuuu

SEQ ID NO: 215 - RNA, 23 nt, Homo sapiens
uagcagcgggaacaguucugcag

SEQ ID NO: 216 - RNA, 22 nt, Homo sapiens
cagcagcaauucauguuuugaa

SEQ ID NO: 217 - RNA, 21 nt, Homo sapiens
uagcagcacagaaauauuggc

SEQ ID NO: 218 - RNA, 22 nt, Homo sapiens
aggcauugacuucucacuagcu

SEQ ID NO: 219 - RNA, 22 nt, Homo sapiens
gugaaauguuuaggaccacuag

SEQ ID NO: 220 - RNA, 22 nt, Homo sapiens
acaguagucugcacauugguua

SEQ ID NO: 221 - RNA, 23 nt, Homo sapiens
cccaguguucagacuaccuguuc

SEQ ID NO: 222 - RNA, 22 nt, Homo sapiens
acaguagucugcacauugguua

SEQ ID NO: 223 - RNA, 23 nt, Homo sapiens
caaagugcuguucgugcagguag

SEQ ID NO: 224 - RNA, 22 nt, Homo sapiens
ugagguaguaaguuguauuguu

SEQ ID NO: 225 - RNA, 24 nt, Homo sapiens
ucccugagacccuuuaaccuguga

SEQ ID NO: 226 - RNA, 22 nt, Homo sapiens
uuugguccccuucaaccagcug

SEQ ID NO: 227 - RNA, 22 nt, Homo sapiens
uuugguccccuucaaccagcua

SEQ ID NO: 228 - RNA, 22 nt, Homo sapiens
ucguaccgugaguaauaaugcg

SEQ ID NO: 229 - RNA, 22 nt, Homo sapiens
uguaacagcaacuccaugugga

SEQ ID NO: 230 - RNA, 23 nt, Homo sapiens
ugucugcccgcaugccugccucu

SEQ ID NO: 231 - RNA, 22 nt, Homo sapiens
uagcagcacaucaugguuuaca

SEQ ID NO: 232 - RNA, 22 nt, Homo sapiens
uccagcaucagugauuuuguug

SEQ ID NO: 233 - RNA, 22 nt, Homo sapiens
uccuucauuccaccggagucug

SEQ ID NO: 234 - RNA, 22 nt, Homo sapiens
ucaccagcccuguguucccuag

SEQ ID NO: 235 - RNA, 23 nt, Homo sapiens
aaggagcuuacaaucuagcuggg

SEQ ID NO: 236 - RNA, 22 nt, Homo sapiens
uggcaguguauuguuagcuggu

SEQ ID NO: 237 - RNA, 22 nt, Homo sapiens
acuggacuuagggucagaaggc

SEQ ID NO: 238 - RNA, 22 nt, Homo sapiens
uuauaaagcaaugagacugauu

SEQ ID NO: 239 - RNA, 23 nt, Homo sapiens
uaaaucccauggugccuucuccu

SEQ ID NO: 240 - RNA, 22 nt, Homo sapiens
aaaaugguucccuuuagagugu

SEQ ID NO: 241 - RNA, 22 nt, Homo sapiens
aggcggggcgccgcgggaccgc

SEQ ID NO: 242 - RNA, 22 nt, Homo sapiens
cagugcaauguuaaaagggcau

SEQ ID NO: 243 - RNA, 22 nt, Homo sapiens
cagugcaaugaugaaagggcau

SEQ ID NO: 244 - RNA, 22 nt, Homo sapiens
ucuucucuguuuuggccaugug

SEQ ID NO: 245 - RNA, 20 nt, Homo sapiens
guccgcucggcgguggccca

SEQ ID NO: 246 - RNA, 23 nt, Homo sapiens
aucgcugcgguugcgagcgcugu

SEQ ID NO: 247 - RNA, 22 nt, Homo sapiens
uggugggccgcagaacaugugc

SEQ ID NO: 248 - RNA, 22 nt, Homo sapiens
uucccuuugucauccuaugccu

SEQ ID NO: 249 - RNA, 21 nt, Homo sapiens
caagucacuagugguuccguu

SEQ ID NO: 250 - RNA, 22 nt, Homo sapiens
agucauuggaggguuugagcag

SEQ ID NO: 251 - RNA, 22 nt, Homo sapiens
uggagugugacaaugguguuug

SEQ ID NO: 252 - RNA, 22 nt, Homo sapiens
uaugugggaugguaaaccgcuu

SEQ ID NO: 253 - RNA, 22 nt, Homo sapiens
ugguuuaccgucccacauacau

SEQ ID NO: 254 - RNA, 22 nt, Homo sapiens
aacccguagauccgaacuugug

SEQ ID NO: 255 - RNA, 23 nt, Homo sapiens
agcugguguugugaaucaggccg

SEQ ID NO: 256 - RNA, 22 nt, Homo sapiens
cagugguuuuacccuaugguag

SEQ ID NO: 257 - RNA, 22 nt, Homo sapiens
uuuguucguucggcucgcguga

SEQ ID NO: 258 - RNA, 23 nt, Homo sapiens
uacugcaucaggaacugauugga

SEQ ID NO: 259 - RNA, 23 nt, Homo sapiens
aaagugcugcgacauuugagcgu

SEQ ID NO: 260 - RNA, 23 nt, Homo sapiens
uuuggcacuagcacauuuuugcu

SEQ ID NO: 261 - RNA, 22 nt, Homo sapiens
ucggauccgucugagcuuggcu

SEQ ID NO: 262 - RNA, 23 nt, Homo sapiens
uauggcuuuucauuccuauguga

SEQ ID NO: 263 - RNA, 21 nt, Homo sapiens
uacaguacugugauaacugaa

SEQ ID NO: 264 - RNA, 20 nt, Homo sapiens
ccucugggcccuuccuccag

SEQ ID NO: 265 - RNA, 23 nt, Homo sapiens
cgcauccccuagggcauuggugu

SEQ ID NO: 266 - RNA, 20 nt, Homo sapiens
acugccccaggugcugcugg

SEQ ID NO: 267 - RNA, 23 nt, Homo sapiens
ucaagagcaauaacgaaaaaugu

SEQ ID NO: 268 - RNA, 22 nt, Homo sapiens
uaacacugucugguaaagaugg

SEQ ID NO: 269 - RNA, 78 nt, Homo sapiens; identical to SEQ ID NO: 143

SEQ ID NO: 270 - RNA, 75 nt, Homo sapiens; identical to SEQ ID NO: 144

SEQ ID NO: 271 - RNA, 72 nt, Homo sapiens
ugucggguagcuuaucagacugauguugacuguugaaucucauggcaacaccagucgaugggcugucugaca

SEQ ID NO: 272 - RNA, 77 nt, Homo sapiens; identical to SEQ ID NO: 104

SEQ ID NO: 273 - RNA, 84 nt, Homo sapiens; identical to SEQ ID NO: 105

SEQ ID NO: 274 - RNA, 71 nt, Homo sapiens
uguucuaaggugcaucuagugcagauagugaaguagauuagcaucuacugcccuaagugcuccuucuggca

SEQ ID NO: 275 - RNA, 87 nt, Homo sapiens
ugagggccccucugcguguucacagcggaccuugauuuaaugucuauacaauuaaggcacgcggugaaugccaagagaggcgccucc

SEQ ID NO: 276 - RNA, 85 nt, Homo sapiens
aggccucucucuccguguucacagcggaccuugauuuaaauguccauacaauuaaggcacgcggugaaugccaagaauggggcug

SEQ ID NO: 277 - RNA, 109 nt, Homo sapiens
aucaagauuagaggcucugcucuccguguucacagcggaccuugauuuaaugucauacaauuaaggcacgcggugaaugccaagagcggagccuacggcugcacuugaa

SEQ ID NO: 278 - RNA, 81 nt, Homo sapiens
cccauuggcauaaacccguagauccgaucuuguggugaaguggaccgcacaagcucgcuucuaugggucugugucagugug

SEQ ID NO: 279 - RNA, 89 nt, Homo sapiens
accaugcuguaguguguguaaacauccuacacucucagcugugagcucaagguggcugggagaggguuguuuacuccuucugccaugga

SEQ ID NO: 280 - RNA, 72 nt, Homo sapiens
agauacuguaaacauccuacacucucagcuguggaaaguaagaaagcugggagaaggcuguuuacucuuucu

SEQ ID NO: 281 - RNA, 86 nt, Homo sapiens
acugcuaacgaaugcucugacuuuauugcacuacuguacuuuacagcuagcagugcaauaguauugucaaagcaucugaaagcagg

SEQ ID NO: 282 - RNA, 88 nt, Homo sapiens; identical to SEQ ID NO: 89

SEQ ID NO: 283 - RNA, 106 nt, Homo sapiens; identical to SEQ ID NO: 87

SEQ ID NO: 284 - RNA, 69 nt, Homo sapiens; identical to SEQ ID NO: 121

SEQ ID NO: 285 - RNA, 69 nt, Homo sapiens; identical to SEQ ID NO: 101

SEQ ID NO: 286 - RNA, 88 nt, Homo sapiens; identical to SEQ ID NO: 110

SEQ ID NO: 287 - RNA, 81 nt, Homo sapiens; identical to SEQ ID NO: 108

SEQ ID NO: 288 - RNA, 81 nt, Homo sapiens; identical to SEQ ID NO: 109

SEQ ID NO: 289 - RNA, 84 nt, Homo sapiens; identical to SEQ ID NO: 81

SEQ ID NO: 290 - RNA, 80 nt, Homo sapiens; identical to SEQ ID NO: 73

SEQ ID NO: 291 - RNA, 72 nt, Homo sapiens; identical to SEQ ID NO: 74

SEQ ID NO: 292 - RNA, 74 nt, Homo sapiens; identical to SEQ ID NO: 75

SEQ ID NO: 293 - RNA, 83 nt, Homo sapiens; identical to SEQ ID NO: 76

SEQ ID NO: 294 - RNA, 119 nt, Homo sapiens; identical to SEQ ID NO: 147

SEQ ID NO: 295 - RNA, 71 nt, Homo sapiens
gcgacuguaaacauccucgacuggaagcugugaagccacagaugggcuuucagucggauguuugcagcugc

SEQ ID NO: 296 - RNA, 84 nt, Homo sapiens; identical to SEQ ID NO: 90

SEQ ID NO: 297 - RNA, 71 nt, Homo sapiens
ugggaaacauacuucuuuauaugcccauauggaccugcuaagcuauggaauguaaagaaguauguaucuca

SEQ ID NO: 298 - RNA, 85 nt, Homo sapiens
accuacucagaguacauacuucuuuauguacccauaugaacauacaaugcuauggaauguaaagaaguauguauuuuugguaggc

SEQ ID NO: 299 - RNA, 110 nt, Homo sapiens
gccgagaccgagugcacagggcucugaccuaugaauugacagccagugcucucgucuccccucuggcugccaauuccauaggucacagguauguucgccucaaugccagc

SEQ ID NO: 300 - RNA, 65 nt, Homo sapiens
cuguuaaugcuaaucgugauagggguuuuugccuccaacugacuccuacauauuagcauuaacag

SEQ ID NO: 301 - RNA, 90 nt, Homo sapiens
ucucaggcugugaccuucucgaggaaagaagcacuuucuguugucugaaagaaaagaaagugcuuccuuucagaggguuacgguuugaga

SEQ ID NO: 302 - RNA, 90 nt, Homo sapiens
ucucagguugugaccuucucgaggaaagaagcacuuucuguugucugaaagaaaagaaagugcuuccuuucagaggguuacgguuugaga

SEQ ID NO: 303 - RNA, 71 nt, Homo sapiens
ggagaggaggcaagaugcuggcauagcuguugaacugggaaccugcuaugccaacauauugccaucuuucc

SEQ ID NO: 304 - RNA, 110 nt, Homo sapiens; identical to SEQ ID NO: 91

SEQ ID NO: 305 - RNA, 110 nt, Homo sapiens; identical to SEQ ID NO: 92

SEQ ID NO: 306 - RNA, 110 nt, Homo sapiens; identical to SEQ ID NO: 93

SEQ ID NO: 307 - RNA, 89 nt, Homo sapiens; identical to SEQ ID NO: 94

SEQ ID NO: 308 - RNA, 110 nt, Homo sapiens; identical to SEQ ID NO: 95

SEQ ID NO: 309 - RNA, 77 nt, Homo sapiens
agucuaguuacuaggcaguguaguuagcugauugcuaauaguaccaaucacuaaccacacggccagguaaaaagauu

SEQ ID NO: 310 - RNA, 84 nt, Homo sapiens
gugcucgguuuguaggcagugucauuagcugauuguacuguggugguuacaaucacuaacuccacugccaucaaaacaaggcac

SEQ ID NO: 311 - RNA, 78 nt, Homo sapiens
uacugcccucggcuucuuuacagugcugccuuguugcauauggaucaagcagcauuguacagggcuaugaaggcauug

SEQ ID NO: 312 - RNA, 78 nt, Homo sapiens
uugugcuuucagcuucuuuacagugcugccuuguagcauucaggucaagcagcauuguacagggcuaugaaagaacca

SEQ ID NO: 313 - RNA, 110 nt, Homo sapiens
acccggcagugccuccaggcgcagggcagccccugcccaccgcacacugcgcugccccagacccacugugcgugugacagcggcugaucugugccugggcagcgcgaccc

SEQ ID NO: 314 - RNA, 89 nt, Homo sapiens
gucagcagugccuuagcagcacguaaauauuggcguuaagauucuaaaauuaucuccaguauuaacugugcugcugaaguaagguugac

SEQ ID NO: 315 - RNA, 81 nt, Homo sapiens
guuccacucuagcagcacguaaauauuggcguagugaaauauauauuaaacaccaauauuacugugcugcuuuagugugac

SEQ ID NO: 316 - RNA, 71 nt, Homo sapiens; identical to SEQ ID NO: 295

SEQ ID NO: 317 - RNA, 71 nt, Homo sapiens; identical to SEQ ID NO: 303

SEQ ID NO: 318 - RNA, 110 nt, Homo sapiens
gcugcuggaagguguagguacccucaauggcucaguagccaguguagauccugucuuucguaaucagcagcuacaucuggcuacugggucucugauggcaucuucuagcu

SEQ ID NO: 319 - RNA, 84 nt, Homo sapiens; identical to SEQ ID NO: 90

SEQ ID NO: 320 - RNA, 84 nt, Homo sapiens; identical to SEQ ID NO: 90

SEQ ID NO: 321 - RNA, 95 nt, Homo sapiens
ccagcucgggcagccguggccaucuuacugggcagcauuggauggagucaggucucuaauacugccugguaaugaugacggcggagcccugcacg

SEQ ID NO: 322 - RNA, 68 nt, Homo sapiens
cccucgucuuacccagcaguguuugggugcgguugggagucucuaauacugccggguaaugauggagg

SEQ ID NO: 323 - RNA, 82 nt, Homo sapiens
ugagcuguuggauucggggccguagcacugucugagagguuuacauuucucacagugaaccggucucuuuuucagcugcuuc

SEQ ID NO: 324 - RNA, 84 nt, Homo sapiens
ugugcagugggaaggggggccgauacacuguacgagagugaguagcaggucucacagugaaccggucucuuucccuacuguguc

SEQ ID NO: 325 - RNA, 71 nt, Homo sapiens
ugcccuagcagcgggaacaguucugcagugagcgaucggugcucugggguauuguuuccgcugccagggua

SEQ ID NO: 326 - RNA, 98 nt, Homo sapiens
cgaggggauacagcagcaauucauguuuugaaguguucuaaaugguucaaaacgugaggcgcugcuauacccccucguggggaagguagaaggugggg

SEQ ID NO: 327 - RNA, 87 nt, Homo sapiens
agcuucccuggcucuagcagcacagaaauauuggcacagggaagcgagucugccaauauuggcugugcugcuccaggcaggguggug

SEQ ID NO: 328 - RNA, 119 nt, Homo sapiens
agucagccuguugaagcuuugaagcuuugaugccaggcauugacuucucacuagcugugaaaguccuagcuaaagagaagucaaugcaugacaucuuguuucaauagauggcuguuuca

SEQ ID NO: 329 - RNA, 110 nt, Homo sapiens
guguuggggacucgcgcgcuggguccagugguucuuaacaguucaacaguucuguagcgcaauugugaaauguuuaggaccacuagacccggcgggcgcggcgacagcga

SEQ ID NO: 330 - RNA, 71 nt, Homo sapiens; identical to SEQ ID NO: 97

SEQ ID NO: 331 - RNA, 71 nt, Homo sapiens; identical to SEQ ID NO: 97

SEQ ID NO: 332 - RNA, 110 nt, Homo sapiens; identical to SEQ ID NO: 98

SEQ ID NO: 333 - RNA, 80 nt, Homo sapiens; identical to SEQ ID NO: 146

SEQ ID NO: 334 - RNA, 119 nt, Homo sapiens; identical to SEQ ID NO: 147

SEQ ID NO: 335 - RNA, 86 nt, Homo sapiens
ugccagucucuaggucccugagacccuuuaaccugugaggacauccagggucacaggugagguucuugggagccuggcgucuggcc

SEQ ID NO: 336 - RNA, 88 nt, Homo sapiens
acaaugcuuugcuagagcugguaaaauggaaccaaaucgccucuucaauggauuugguccccuucaaccagcuguagcuaugcauuga

SEQ ID NO: 337 - RNA, 102 nt, Homo sapiens
gggagccaaaugcuuugcuagagcugguaaaauggaaccaaaucgacuguccaauggauuugguccccuucaaccagcuguagcugugcauugauggcgccg

SEQ ID NO: 338 - RNA, 119 nt, Homo sapiens
ccucagaagaaagaugcccccugcucuggcuggucaaacggaaccaaguccgucuuccugagagguuugguccccuucaaccagcuacagcagggcuggcaaugcccaguccuuggaga

SEQ ID NO: 339 - RNA, 85 nt, Homo sapiens
cgcuggcgacgggacauuauuacuuuugguacgcgcugugacacuucaaacucguaccgugaguaauaaugcgccguccacggca

SEQ ID NO: 340 - RNA, 85 nt, Homo sapiens
augguguuaucaaguguaacagcaacuccauguggacuguguaccaauuuccaguggagaugcuguuacuuuugaugguuaccaa

SEQ ID NO: 341 - RNA, 85 nt, Homo sapiens
ugguucccgcccccuguaacagcaacuccauguggaagugcccacugguuccaguggggcugcuguuaucuggggcgagggccag

SEQ ID NO: 342 - RNA, 95 nt, Homo sapiens
ggucucuguguugggcgucugucugcccgcaugccugccucucuguugcucugaaggaggcaggggcugggccugcagcugccugggcagagcgg

SEQ ID NO: 343 - RNA, 98 nt, Homo sapiens
uugaggccuuaaaguacuguagcagcacaucaugguuuacaugcuacagucaagaugcgaaucauuauuugcugcucuagaaauuuaaggaaauucau

SEQ ID NO: 344 - RNA, 67 nt, Homo sapiens
ucuccaacaauauccuggugcugagugaugacucaggcgacuccagcaucagugauuuuguugaaga

SEQ ID NO: 345 - RNA, 110 nt, Homo sapiens
aaagauccucagacaauccaugugcuucucuuguccuucauuccaccggagucugucucauacccaaccagauuucaguggagugaaguucaggaggcauggagcugaca

SEQ ID NO: 346 - RNA, 75 nt, Homo sapiens
gugagggcaugcaggccuggauggggcagcugggaugguccaaaaggguggccucaccagcccuguguucccuag

SEQ ID NO: 347 - RNA, 88 nt, Homo sapiens
aacugcccucaaggagcuuacaaucuagcuggggguaaaugacuugcacaugaacacaacuagacugugagcuucuagagggcaggga

SEQ ID NO: 348 - RNA, 91 nt, Homo sapiens
cugugugugaugagcuggcaguguauuguuagcugguugaauaugugaauggcaucggcuaacaugcaacugcugucuuauugcauauaca

SEQ ID NO: 349 - RNA, 90 nt, Homo sapiens
gagagaagcacuggacuuagggucagaaggccugagucucucugcugcagaugggcucucugucccugagccaagcuuuguccucccugg

SEQ ID NO: 350 - RNA, 95 nt, Homo sapiens
uuguaccuggugugauuauaaagcaaugagacugauugucauaugucguuugugggauccgucucaguuacuuuauagccauaccugguaucuua

SEQ ID NO: 351 - RNA, 83 nt, Homo sapiens
gcccuagcuugguucuaaaucccauggugccuucuccuugggaaaaacagagaaggcacuaugagauuuagaaucaaguuagg

SEQ ID NO: 352 - RNA, 87 nt, Homo sapiens
ucucaggcugugucccucuagagggaagcgcuuucuguugucugaaagaaaagaaaaugguucccuuuagaguguuacgcuuugaga

SEQ ID NO: 353 - RNA, 93 nt, Homo sapiens
ccuuccggcgucccaggcggggcgccgcgggaccgcccucgugucuguggcggugggaucccgcggccguguuuuccugguggcccggccaug

SEQ ID NO: 354 - RNA, 89 nt, Homo sapiens
ugcugcuggccagagcucuuuucacauugugcuacugucugcaccugucacuagcagugcaauguuaaaagggcauuggccguguagug

SEQ ID NO: 355 - RNA, 82 nt, Homo sapiens
ggccugcccgacacucuuucccuguugcacuacuauaggccgcugggaagcagugcaaugaugaaagggcaucggucagguc

SEQ ID NO: 356 - RNA, 86 nt, Homo sapiens
auuaggagaguaucuucucuguuuuggccauguguguacucacagccccucacacauggccgaaacagagaaguuacuuuccuaau

SEQ ID NO: 357 - RNA, 95 nt, Homo sapiens
gucgaggccguggcccggaaguggucggggccgcugcgggcggaagggcgccugugcuucguccgcucggcgguggcccagccaggcccgcggga

SEQ ID NO: 358 - RNA, 98 nt, Homo sapiens
uggccgacggggcgcgcgcggccuggaggggcggggcggacgcagagccgcguuuagucuaucgcugcgguugcgagcgcuguagggagccugugcug

SEQ ID NO: 359 - RNA, 81 nt, Homo sapiens
ggguaaguggaaagauggugggccgcagaacaugugcugaguucgugccauaugucugcugaccaucaccuuuagaagccc

SEQ ID NO: 360 - RNA, 110 nt, Homo sapiens
ggcuacagucuuucuucaugugacucguggacuucccuuugucauccuaugccugagaauauaugaaggaggcugggaaggcaaagggacguucaauugucaucacuggc

SEQ ID NO: 361 - RNA, 81 nt, Homo sapiens
gggcuuucaagucacuagugguuccguuuaguagaugauugugcauuguuucaaaauggugcccuagugacuacaaagccc

SEQ ID NO: 362 - RNA, 97 nt, Homo sapiens
uuagguaauuccuccacucaaaacccuucagugacuuccaugacaugaaauaggaagucauuggaggguuugagcagaggaaugaccuguuuuaaaa

SEQ ID NO: 363 - RNA, 85 nt, Homo sapiens
ccuuagcagagcuguggagugugacaaugguguuugugucuaaacuaucaaacgccauuaucacacuaaauagcuacugcuaggc

SEQ ID NO: 364 - RNA, 63 nt, Homo sapiens
aagaaaugguuuaccgucccacauacauuuugaauauguaugugggaugguaaaccgcuucuu

SEQ ID NO: 365 - RNA, 80 nt, Homo sapiens
ccuguugccacaaacccguagauccgaacuugugguauuaguccgcacaagcuuguaucuauagguaugugucuguuagg

SEQ ID NO: 366 - RNA, 99 nt, Homo sapiens
cccuggcauggugugguggggcagcugguguugugaaucaggccguugccaaucagagaacggcuacuucacaacaccagggccacaccacacuacagg

SEQ ID NO: 367 - RNA, 100 nt, Homo sapiens
ugugucucucucuguguccugccagugguuuuacccuaugguagguuacgucaugcuguucuaccacaggguagaaccacggacaggauaccggggcacc

SEQ ID NO: 368 - RNA, 64 nt, Homo sapiens
ccccgcgacgagccccucgcacaaaccggaccugagcguuuuguucguucggcucgcgugaggc

SEQ ID NO: 369 - RNA, 110 nt, Homo sapiens
aguauaauuauuacauaguuuuugaugucgcagauacugcaucaggaacugauuggauaagaaucagucaccaucaguuccuaaugcauugccuucagcaucuaaacaag

SEQ ID NO: 370 - RNA, 67 nt, Homo sapiens; identical to SEQ ID NO: 120

SEQ ID NO: 371 - RNA, 78 nt, Homo sapiens
uggccgauuuuggcacuagcacauuuuugcuugugucucuccgcucugagcaaucaugugcagugccaauaugggaaa

SEQ ID NO: 372 - RNA, 97 nt, Homo sapiens
ugugaucacugucuccagccugcugaagcucagagggcucugauucagaaagaucaucggauccgucugagcuuggcuggucggaagucucaucauc

SEQ ID NO: 373 - RNA, 97 nt, Homo sapiens
cacucugcuguggccuauggcuuuucauuccuaugugauugcugucccaaacucauguagggcuaaaagccaugggcuacagugaggggcgagcucc

SEQ ID NO: 374 - RNA, 75 nt, Homo sapiens
ugcccuggcucaguuaucacagugcugaugcugucuauucuaaagguacaguacugugauaacugaaggauggca

SEQ ID NO: 375 - RNA, 79 nt, Homo sapiens
acuguccuuuuucgguuaucaugguaccgaugcuguauaucugaaagguacaguacugugauaacugaagaaugguggu

SEQ ID NO: 376 - RNA, 95 nt, Homo sapiens; identical to SEQ ID NO: 114

SEQ ID NO: 377 - RNA, 83 nt, Homo sapiens
cugacuaugccuccccgcauccccuagggcauugguguaaagcuggagacccacugccccaggugcugcuggggguuguaguc

SEQ ID NO: 378 - RNA, 83 nt, Homo sapiens; identical to SEQ ID NO: 377

SEQ ID NO: 379 - RNA, 94 nt, Homo sapiens
uguuuugagcgggggucaagagcaauaacgaaaaauguuugucauaaaccguuuuucauuauugcuccugaccuccucucauuugcuauauuca

SEQ ID NO: 380 - RNA, 95 nt, Homo sapiens
cggccggcccuggguccaucuuccaguacaguguuggauggucuaauugugaagcuccuaacacugucugguaaagauggcucccggguggguuc

SEQ ID NO: 381 - RNA, 17 nt, Homo sapiens
gugggggagaggcuguc

SEQ ID NO: 382 - RNA, 22 nt, Homo sapiens
ugcaacgaaccugagccacuga

SEQ ID NO: 383 - RNA, 22 nt, Homo sapiens
uagguuauccguguugccuucg

SEQ ID NO: 384 - RNA, 21 nt, Homo sapiens
gugccagcugcagugggggag

SEQ ID NO: 385 - RNA, 20 nt, Homo sapiens
guccgcucggcgguggccca

SEQ ID NO: 386 - RNA, 23 nt, Homo sapiens
ccaguuaccgcuuccgcuaccgc

SEQ ID NO: 387 - RNA, 17 nt, Homo sapiens
acauugccagggaguuu

SEQ ID NO: 388 - RNA, 22 nt, Homo sapiens
uugcauagucacaaaagugauc

SEQ ID NO: 389 - RNA, 17 nt, Homo sapiens
uugucugcugaguuucc

SEQ ID NO: 390 - RNA, 23 nt, Homo sapiens
agguuacccgagcaacuuugcau

SEQ ID NO: 391 - RNA, 22 nt, Homo sapiens
ugggucuuugcgggcgagauga

SEQ ID NO: 392 - RNA, 19 nt, Homo sapiens
aagugugcagggcacuggu

SEQ ID NO: 393 - RNA, 22 nt, Homo sapiens
uaaugccccuaaaaauccuuau

SEQ ID NO: 394 - RNA, 23 nt, Homo sapiens
uaauccuugcuaccugggugaga

SEQ ID NO: 395 - RNA, 22 nt, Homo sapiens
aguggggaacccuuccaugagg

SEQ ID NO: 396 - RNA, 22 nt, Homo sapiens
acaguagucugcacauugguua

SEQ ID NO: 397 - RNA, 22 nt, Homo sapiens
acaguagucugcacauugguua

SEQ ID NO: 398 - RNA, 23 nt, Homo sapiens
cccaguguucagacuaccuguuc

SEQ ID NO: 399 - RNA, 23 nt, Homo sapiens
cccaguguucagacuaccuguuc

SEQ ID NO: 400 - RNA, 21 nt, Homo sapiens
auuugugcuuggcucugucac

SEQ ID NO: 401 - RNA, 23 nt, Homo sapiens
aaagugcugcgacauuugagcgu

SEQ ID NO: 402 - RNA, 23 nt, Homo sapiens
gaagugcuucgauuuuggggugu

SEQ ID NO: 403 - RNA, 22 nt, Homo sapiens
ucuucucuguuuuggccaugug

SEQ ID NO: 404 - RNA, 22 nt, Homo sapiens
uggguggucuggagauuugugc

SEQ ID NO: 405 - RNA, 23 nt, Homo sapiens
uaaggugcaucuagugcagauag

SEQ ID NO: 406 - RNA, 23 nt, Homo sapiens
gagggucuugggagggaugugac

SEQ ID NO: 407 - RNA, 21 nt, Homo sapiens
agaggauacccuuuguauguu

SEQ ID NO: 408 - RNA, 20 nt, Homo sapiens
uaaagagcccuguggagaca

SEQ ID NO: 409 - RNA, 22 nt, Homo sapiens
aacuggcccucaaagucccgcu

SEQ ID NO: 410 - RNA, 20 nt, Homo sapiens
cuuccucgucugucugcccc

SEQ ID NO: 411 - RNA, 21 nt, Homo sapiens
uuaauaucggacaaccauugu

SEQ ID NO: 412 - RNA, 22 nt, Homo sapiens
gccugcugggguggaaccuggu

SEQ ID NO: 413 - RNA, 22 nt, Homo sapiens
caaaaaccacaguuucuuuugc

SEQ ID NO: 414 - RNA, 80 nt, Homo sapiens
ccucugugagaaagggugugggggagaggcugucuugugucuguaaguaugccaaacuuauuuuccccaaggcagaggga

SEQ ID NO: 415 - RNA, 79 nt, Homo sapiens
ccuuaauccuugcaacgaaccugagccacugauucaguaaaauacucaguggcacauguuuguugugagggucaaaaga

SEQ ID NO: 416 - RNA, 84 nt, Homo sapiens
gugguacuugaagauagguuauccguguugccuucgcuuuauuugugacgaaucauacacgguugaccuauuuuucaguaccaa

SEQ ID NO: 417 - RNA, 83 nt, Homo sapiens
ccugcugcagaggugccagcugcagugggggaggcacugccagggcugcccacucugcuuagccagcaggugccaagaacagg

SEQ ID NO: 418 - RNA, 95 nt, Homo sapiens; identical to SEQ ID NO: 357

SEQ ID NO: 419 - RNA, 91 nt, Homo sapiens
ggcgggggcgcgggcggcaguggcgggagcggccccucggccauccuccgucugcccaguuaccgcuuccgcuaccgccgccgcucccgcu

SEQ ID NO: 420 - RNA, 65 nt, Homo sapiens
aaaaggcgagacauugccagggaguuuauuuuguagcucucuugauaaaauguuuuagcaaacac

SEQ ID NO: 421 - RNA, 90 nt, Homo sapiens
cucacagcugccagugucauuuuugugaucugcagcuaguauucucacuccaguugcauagucacaaaagugaucauuggcagguguggc

SEQ ID NO: 422 - RNA, 87 nt, Homo sapiens
agcgguggccagugucauuuuugugauguugcagcuaguaauaugagcccaguugcauagucacaaaagugaucauuggaaacugug

SEQ ID NO: 423 - RNA, 67 nt, Homo sapiens
auggagguggagagucaucagcagcacugagcaggcaguguugucugcugaguuuccacgucauuug

SEQ ID NO: 424 - RNA, 79 nt, Homo sapiens
ugguacucggggagagguuacccgagcaacuuugcaucuggacgacgaauguugcucggugaaccccuuuucgguauca

SEQ ID NO: 425 - RNA, 88 nt, Homo sapiens
cgaggaugggagcugagggcugggucuuugcgggcgagaugagggugucggaucaacuggccuacaaagucccaguucucggcccccg

SEQ ID NO: 426 - RNA, 94 nt, Homo sapiens
aucacagacaccuccaagugugcagggcacuggugggggccggggcaggcccagcgaaagugcaggaccuggcacuuagucggaagugagggug

SEQ ID NO: 427 - RNA, 87 nt, Homo sapiens
accgcagggaaaaugagggacuuuugggggcagauguguuuccauuccacuaucauaaugccccuaaaaauccuuauugcucuugca

SEQ ID NO: 428 - RNA, 84 nt, Homo sapiens
gcucccccucucuaauccuugcuaccugggugagagugcugucugaaugcaaugcaccugggcaaggauucugagagcgagagc

SEQ ID NO: 429 - RNA, 84 nt, Homo sapiens
uugacuuagcuggguaguggggaacccuuccaugaggaguagaacacuccuuaugcaagauucccuucuaccuggcuggguugg

SEQ ID NO: 430 - RNA, 71 nt, Homo sapiens; identical to SEQ ID NO: 97

SEQ ID NO: 431 - RNA, 110 nt, Homo sapiens
aggaagcuucuggagauccugcuccgucgccccaguguucagacuaccuguucaggacaaugccguuguacaguagucugcacauugguuagacugggcaagggagagca

SEQ ID NO: 432 - RNA, 71 nt, Homo sapiens; identical to SEQ ID NO: 97

SEQ ID NO: 433 - RNA, 110 nt, Homo sapiens; identical to SEQ ID NO: 431

SEQ ID NO: 434 - RNA, 91 nt, Homo sapiens; identical to SEQ ID NO: 102

SEQ ID NO: 435 - RNA, 67 nt, Homo sapiens; identical to SEQ ID NO: 120

SEQ ID NO: 436 - RNA, 69 nt, Homo sapiens; identical to SEQ ID NO: 121

SEQ ID NO: 437 - RNA, 86 nt, Homo sapiens; identical to SEQ ID NO: 356

SEQ ID NO: 438 - RNA, 71 nt, Homo sapiens
agguuguucuggguggucuggagauuugugcagcuuguaccugcacaaaucuccggaccacuuagucuuua

SEQ ID NO: 439 - RNA, 71 nt, Homo sapiens; identical to SEQ ID NO: 274

SEQ ID NO: 440 - RNA, 97 nt, Homo sapiens
gggacuugucacugccugucuccucccucuccagcagcgacuggauucuggaguccaucuagagggucuugggagggaugugacuguugggaagccc

SEQ ID NO: 441 - RNA, 86 nt, Homo sapiens
uuugguacuugaagagaggauacccuuuguauguucacuugauuaauggcgaauauacagggggagacucuuauuugcguaucaaa

SEQ ID NO: 442 - RNA, 86 nt, Homo sapiens
uuugguacuuaaagagaggauacccuuuguauguucacuugauuaauggcgaauauacagggggagacucucauuugcguaucaaa

SEQ ID NO: 443 - RNA, 83 nt, Homo sapiens
ccccagcuagguaaagagcccuguggagacaccuggauucagagaacaugucuccacugagcacuugggccuugauggcggcu

SEQ ID NO: 444 - RNA, 83 nt, Homo sapiens
guggucucagaaucgggguuuugagggcgagaugaguuuauguuuuauccaacuggcccucaaagucccgcuuuuggggucau

SEQ ID NO: 445 - RNA, 83 nt, Homo sapiens
gugagugggagccccagugugugguuggggccauggcgggugggcagcccagccucugagccuuccucgucugucugccccag

SEQ ID NO: 446 - RNA, 79 nt, Homo sapiens
gugcuuaaagaauggcuguccguaguauggucucuauauuuaugaugauuaauaucggacaaccauuguuuuaguaucc

SEQ ID NO: 447 - RNA, 75 nt, Homo sapiens
agacagagaagccaggucacgucucugcaguuacacagcucacgagugccugcugggguggaaccuggucugucu

SEQ ID NO: 448 - RNA, 97 nt, Homo sapiens
aaacaaguuauauuagguuggugcaaaaguaauugugguuuuugccuguaaaaguaauggcaaaaaccacaguuucuuuugcaccagacuaauaaag

SEQ ID NO: 449 - RNA, 23 nt, Homo sapiens
caaagugcucauagugcagguag

SEQ ID NO: 450 - RNA, 23 nt, Homo sapiens
uaaggugcaucuagugcagauag

SEQ ID NO: 451 - RNA, 23 nt, Homo sapiens
caaagugcuuacagugcagguag

SEQ ID NO: 452 - RNA, 22 nt, Homo sapiens
uaacacugucugguaaagaugg

SEQ ID NO: 453 - RNA, 22 nt, Homo sapiens
acuuuaacauggaagugcuuuc

SEQ ID NO: 454 - RNA, 21 nt, Homo sapiens
uacaguacugugauaacugaa

SEQ ID NO: 455 - RNA, 21 nt, Homo sapiens
cauuauuacuuuugguacgcg

SEQ ID NO: 456 - RNA, 22 nt, Homo sapiens
ugagaacugaauuccauggguu

SEQ ID NO: 457 - RNA, 22 nt, Homo sapiens
ugagaacugaauuccauaggcu

SEQ ID NO: 458 - RNA, 22 nt, Homo sapiens
uucaaguaauccaggauaggcu

SEQ ID NO: 459 - RNA, 22 nt, Homo sapiens
uagcaccaucugaaaucgguua

SEQ ID NO: 460 - RNA, 22 nt, Homo sapiens
uaacagucuacagccauggucg

SEQ ID NO: 461 - RNA, 23 nt, Homo sapiens
ucuuugguuaucuagcuguauga

SEQ ID NO: 462 - RNA, 22 nt, Homo sapiens
ugagaacugaauuccauggguu

SEQ ID NO: 463 - RNA, 23 nt, Homo sapiens
uacccuguagaaccgaauuugug

SEQ ID NO: 464 - RNA, 21 nt, Homo sapiens
agcuacaucuggcuacugggu

SEQ ID NO: 465 - RNA, 22 nt, Homo sapiens
aacuggcccucaaagucccgcu

SEQ ID NO: 466 - RNA, 23 nt, Homo sapiens
agcuacauugucugcuggguuuc

SEQ ID NO: 467 - RNA, 23 nt, Homo sapiens
uauggcuuuuuauuccuauguga

SEQ ID NO: 468 - RNA, 23 nt, Homo sapiens
ucuggcuccgugucuucacuccc

SEQ ID NO: 469 - RNA, 23 nt, Homo sapiens
cccaguguucagacuaccuguuc

SEQ ID NO: 470 - RNA, 23 nt, Homo sapiens
acuuaaacguggauguacuugcu

SEQ ID NO: 471 - RNA, 22 nt, Homo sapiens
uuuaacauggggguaccugcug

SEQ ID NO: 472 - RNA, 23 nt, Homo sapiens
uaagugcuuccauguuugagugu

SEQ ID NO: 473 - RNA, 21 nt, Homo sapiens
aauaauacaugguugaucuuu

SEQ ID NO: 474 - RNA, 22 nt, Homo sapiens
gccugcugggguggaaccuggu

SEQ ID NO: 475 - RNA, 22 nt, Homo sapiens
ugagguaguagguuguauaguu

SEQ ID NO: 476 - RNA, 22 nt, Homo sapiens
ugagguaguagguugugugguu

SEQ ID NO: 477 - RNA, 23 nt, Homo sapiens
uacccuguagaaccgaauuugug

SEQ ID NO: 478 - RNA, 21 nt, Homo sapiens
aucacauugccagggauuucc

SEQ ID NO: 479 - RNA, 21 nt, Homo sapiens
aucacauugccagggauuacc

SEQ ID NO: 480 - RNA, 22 nt, Homo sapiens
uauugcacauuacuaaguugca

SEQ ID NO: 481 - RNA, 23 nt, Homo sapiens
aagugccgccaucuuuugagugu

SEQ ID NO: 482 - RNA, 22 nt, Homo sapiens
ugugacugguugaccagagggg

SEQ ID NO: 483 - RNA, 21 nt, Homo sapiens
ugauuguccaaacgcaauucu

SEQ ID NO: 484 - RNA, 22 nt, Homo sapiens
uagguuauccguguugccuucg

SEQ ID NO: 485 - RNA, 23 nt, Homo sapiens
uuaaugcuaaucgugauaggggu

SEQ ID NO: 486 - RNA, 21 nt, Homo sapiens
gugcauuguaguugcauugca

SEQ ID NO: 487 - RNA, 22 nt, Homo sapiens
ucggauccgucugagcuuggcu

SEQ ID NO: 488 - RNA, 22 nt, Homo sapiens
uaacagucuacagccauggucg

SEQ ID NO: 489 - RNA, 23 nt, Homo sapiens
uuauugcuuaagaauacgcguag

SEQ ID NO: 490 - RNA, 23 nt, Homo sapiens
uguaguguuuccuacuuuaugga

SEQ ID NO: 491 - RNA, 22 nt, Homo sapiens
ucccugagacccuaacuuguga

SEQ ID NO: 492 - RNA, 22 nt, Homo sapiens
uugcauagucacaaaagugauc

SEQ ID NO: 493 - RNA, 23 nt, Homo sapiens
aacauucaacgcugucggugagu

SEQ ID NO: 494 - RNA, 22 nt, Homo sapiens
ugagguaguagguuguauaguu

SEQ ID NO: 495 - RNA, 22 nt, Homo sapiens
ugagguaguagguugugugguu

SEQ ID NO: 496 - RNA, 21 nt, Homo sapiens
aacauagaggaaauuccacgu

SEQ ID NO: 497 - RNA, 21 nt, Homo sapiens
uugugcuugaucuaaccaugu

SEQ ID NO: 498 - RNA, 20 nt, Homo sapiens
uaaggcacgcggugaaugcc

SEQ ID NO: 499 - RNA, 24 nt, Homo sapiens
ucccugagacccuuuaaccuguga

SEQ ID NO: 500 - RNA, 23 nt, Homo sapiens
uaaagugcuuauagugcagguag

SEQ ID NO: 501 - RNA, 22 nt, Homo sapiens
cagugcaauguuaaaagggcau

SEQ ID NO: 502 - RNA, 23 nt, Homo sapiens
aaagugcugcgacauuugagcgu

SEQ ID NO: 503 - RNA, 22 nt, Homo sapiens
acucaaaaugggggcgcuuucc

SEQ ID NO: 504 - RNA, 22 nt, Homo sapiens
ugucaguuugucaaauacccca